(19) United States
(12) Patent Application Publication
(10) Pub. No.: US 2024/0361301 A1
(43) Pub. Date: Oct. 31, 2024

(54) METHODS AND COMPOSITIONS FOR MODULATING GOBLET CELLS AND FOR MUCO-OBSTRUCTIVE DISEASES

(71) Applicant: Flagship Pioneering Innovations VI, LLC, Cambridge, MA (US)

(72) Inventors: Morag Helen STEWART, Boston, MA (US); Angeliki CHALKIADAKI, Brookline, MA (US); Yuge JI, Cambridge, MA (US); Fabian Alexander WOLF, Munich (DE); Nicholas McCartney PLUGIS, Boston, MA (US); Poorya HOSSEINI, Bostom, MA (US); Inna LIPCHINA, Newton, MA (US); Sophie Andrea TRITSCHLER, Munich (DE)

(21) Appl. No.: 18/687,654

(22) PCT Filed: Sep. 9, 2022
(86) PCT No.: PCT/US2022/043059

§ 371 Date: Feb. 28, 2024

Related U.S. Application Data
(60) Provisional application No. 63/276,158, filed on Nov. 5, 2021.
(60) Provisional application No. 63/244,028, filed on Sep. 14, 2021.
(60) Provisional application No. 63/242,296, filed on Sep. 9, 2021.

(51) Int. Cl. G01N 33/50; A61K 45/06; C12N 5/071

(52) U.S. Cl. CPC G01N 33/5088; A61K 45/06; C12N 5/0679; G01N 33/5023; C12N 2503/04; C12N 2533/90

(57) ABSTRACT

The present disclosure relates, inter alia, to perturbagens and methods for directing a change in the cell state of an intestinal stem cell and/or a basal cell. It also relates to methods for increasing a quantity of goblet progenitors, goblet cells, Paneth cells, and/or enteroendocrine cells or immediate progenitors thereof and/or the ratios thereof, and methods for decreasing the function and quantity of goblet cells or immediate progenitors thereof. Further, the present disclosure relates to methods for treating diseases or disorders characterized by, at least, abnormal function, abnormal ratios and/or abnormal numbers of goblet progenitors, goblet cells, Paneth cells, and/or enteroendocrine cells, or immediate progenitors thereof.

### CROSS-REFERENCE TO RELATED APPLICATIONS

This application claims priority to and benefit of U.S. Provisional Patent Application No. 63/242,296 filed on Sep. 9, 2021, U.S. Provisional Patent Application No. 63/244,028 filed on Sep. 14, 2021, and U.S. Provisional Patent Application No. 63/276,158 filed on Nov. 5, 2021, the contents of all of which are hereby incorporated by reference in their entireties.

### BACKGROUND

An understanding of cellular mechanisms relating to development of goblet cells and their progenitors, as well as methods and agents for directing changes in development of these cells, may be useful for treating diseases or disorders, including those underscored by abnormal amounts or ratio of various cell types in the goblet cell lineage.

Currently, there is an unmet need for such methods and agents that can be used for the treatment of such diseases and disorders.

### SUMMARY

In certain aspects, the present disclosure is related to a method for directing a change in cell state of a progenitor cell comprising: contacting a population of cells comprising a progenitor cell with at least one perturbagen selected from Table 2, or a variant thereof; wherein the at least one perturbagen is capable of altering a gene signature in the progenitor cell; and wherein the progenitor cell is an intestinal stem cell.

In certain aspects, the present disclosure is related to a method for directing a change in cell state of a progenitor cell, comprising: contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In certain aspects, the present disclosure is related to a method for directing a change in cell state of a progenitor cell, comprising: contacting a population of cells comprising a progenitor cell with at least one perturbagen selected from Table 2, or a variant thereof, and capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In some embodiments, altering the gene signature comprises a change in expression and/or activity of one or more genes in the progenitor cell of a network module designated in the network module column of Table 1.

In some embodiments, the change in cell state provides an increase in the number of one or more of goblet progenitors, goblet cells, Paneth cells, and enteroendocrine cells.

In some embodiments, the change in cell state provides an increase in the number of goblet cells.

In some embodiments, the increase in the number of goblet cells is relative to the number of goblets cells obtained from a population of progenitor cells that is not contacted with the at least one perturbagen or relative to the number of goblets cells obtained from a population of progenitor cells prior to contacting with the at least one perturbagen.

In some embodiments, the number of progenitor cells is decreased.

In some embodiments, the decrease in the number of progenitor cells is relative to the number of progenitor cells in a population of progenitor cells that is not contacted with the at least one perturbagen or relative to the number of progenitor cells in the population prior to contacting with the at least one perturbagen.

In some embodiments, the number of progenitor cells is increased.

In some embodiments, the increase in the number of progenitor cells is relative to the number of progenitor cells in a population of progenitor cells that is not contacted with the at least one perturbagen or relative to the number of progenitor cells in the population prior to contacting with the at least one perturbagen.

In some embodiments, the number of goblet progenitors, goblet cells, Paneth cells, and/or enteroendocrine cells is increased after contacting the population of cells comprising a progenitor cell with the at least one perturbagen.

In some embodiments, the number of goblet progenitors, Paneth cells, enteroendocrine cells, enterocyte progenitors, and/or enterocytes is decreased.

In some embodiments, the number of goblet progenitors, goblet cells, Paneth cells, and/or enteroendocrine cells is increased.

In some embodiments, the at least one perturbagen selected from Table 2, or a variant thereof, comprises at least 2, at least 3, at least 4, at least 5, at least 6, at least 7, at least 8, at least 9, at least 10, or at least 11 perturbagens selected from Table 2, or variants thereof.

In some embodiments, the one or more genes are selected from the genes designated as an “up” gene in the gene directionality column of Table 1 comprises 2 or more, 3 or more, 4 or more, 5 or more, 6 or more, 7 or more, 8 or more, 9 or more, 10 genes or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, or 58 or more genes selected from the genes designated as an “up” gene in the gene directionality column of Table 1.

In some embodiments, the one or more genes selected from Table 1 comprises at least one of BIRC5, CCNB1, UBE2C, CDC20, CCNA2, S100A13, PMM2, GADD45B, GADD45A, RAP1GAP, TM9SF2, TMED10, STXBP1, GALE, KDELR2, ADGRE5, MTHFD2, FHL2, PYCR1, MVP, BAMBI, BAD, HMGCS1, ATP6V0B, FDFT1, HMGCR, EBP, ACLY, FGFR2, STAP2, XBP1, GFPT1, CDKN1A, HYOU1, ACBD3, COPB2, HERPUD1, NUCB2, CDC25B, BACE2, RGS2, RPN1, RAB27A, SLC35A1, RNH1, C2CD2L, ARHGAP1, SLC35B1, CRELD2, UFM1, ARFIP2, KIT, FAIM, NPDC1, TIMM17B, PDIA5, TM9SF3, and EDEM1.

In some embodiments, the one or more genes are selected from the genes designated as a “down” gene in the gene directionality column of Table 1 comprises 2 or more, 3 or more, 4 or more, 5 or more, 6 or more, 7 or more, 8 or more, 9 or more, 10 genes or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or ore, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or ore, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, or 81 or more genes selected from the genes designated as a “down” gene in the gene directionality column of Table 1.

In some embodiments, the one or more genes selected from Table 1 comprises at least one of MIF, TRAP1, CETN3, CHEK2, RAE1, HES1, HADH, RFC2, MCM3, PCNA, HAT1, TOPBP1, EED, DDB2, PKIG, SNX7, DYRK3, CCNH, PHGDH, GLRX, CHP1, NR3C1, TSC22D3, EIF5, FOXO3, SOX4, H2AFV, GRB7, SNX6, NOTCH1, NET1, CD44, VDAC1, CREG1, PRSS23, FKBP4, MBNL1, MYC, MRPL12, CD320, G3BP1, CBR1, IFRD2, GRWD1, RPS5, TSPAN3, ADCK3, OXCT1, GPER1, PDGFA, TRIM2, APP, UGDH, PSME1, MTA1, RPA2, PGRMC1, PSIP1, HSPD1, PAICS, SDHB, NOLC1, ATP1B1, PLA2G4A, TOMM70A, ADH5, MRPL19, AARS, SOCS2, HEBP1, TIMM9, NIPSNAP1, OXA1L, MSRA, ARIDSB, BZW2, EDN1, POLR1C, CDK4, DCTD, and HSD17B10.

In some embodiments, the change in cells state provides one or more of: (a) increased secretion of mucus by a goblet cell and (b) increased synthesis of one or more mucins, optionally selected from MUC2, MUC1, MUC3, or MUC17), trefoil factor peptides (TFF), resistin-like moleculea β (RELMβ), and Fc-γ binding protein (FcγBP).

In certain aspects, the present disclosure is related to a method for inhibiting a change in cell state of a progenitor cell comprising: contacting a population of cells comprising a progenitor cell with at least one perturbagen selected from Table 4, or a variant thereof, wherein the at least one perturbagen is capable of altering a gene signature in the progenitor cell; and wherein the progenitor cell is a basal cell.

In certain aspects, the present disclosure is related to a method for inhibiting a change in cell state of a progenitor cell, comprising: contacting a population of cells comprising a progenitor cell with at least one perturbagen capable of altering a gene signature in the progenitor cell, wherein altering the gene signature comprises a decrease in expression and/or activity in the progenitor cell of one or more genes designated as a “down” gene in the gene directionality column of Table 3 and wherein the progenitor cell is a basal cell.

In certain aspects, the present disclosure is related to a method for inhibiting a change in cell state of a progenitor cell, comprising: contacting a population of cells comprising a progenitor cell with at least one perturbagen selected from Table 4, or a variant thereof, and capable of altering a gene signature in the progenitor cell, wherein altering the gene signature comprises a decrease in expression and/or activity in the progenitor cell of one or more genes selected from Table 4 and wherein the progenitor cell is a basal cell.

In some embodiments, altering the gene signature comprises an activation of a network module designated in the network module column of Table 3.

In some embodiments, inhibiting the change in cell state provides: i) a decrease in the number of goblet cells; ii) an increase in the number of club cells; and/or iii) an increase in the number of ciliated cells.

In some embodiments, the decrease in the number of goblet cells is relative to the number of basal cells or basal luminal precursor cells or club cells or ciliated cells or neuroendocrine cells obtained from a population of progenitor cells i) that is not contacted with the at least one perturbagen or ii) prior to contacting with the at least one perturbagen.

In some embodiments, the increase in the number of club cells is relative to the number of basal cells, goblet cells, basal luminal precursor cells, ciliated cells, and/or neuroendocrine cells obtained from a population of progenitor cells i) that is not contacted with the at least one perturbagen or ii) prior to contacting with the at least one perturbagen.

In some embodiments, the increase in the number of ciliated cells is relative to the number of basal cells, goblet cells, basal luminal precursor cells, club cells, and/or neuroendocrine cells obtained from a population of progenitor cells i) that is not contacted with the at least one perturbagen or ii) prior to contacting with the at least one perturbagen.

In some embodiments, the number of basal cells is increased.

In some embodiments, the increase in the number of basal cells is relative to the number of basal cells in a population of basal cells that is not contacted with the at least one perturbagen or relative to the number of basal cells in the population prior to contacting with the at least one perturbagen.

In some embodiments, the number of basal luminal precursor cells, and/or goblet cells is decreased after contacting the population of cells comprising a basal cell with the at least one perturbagen.

In some embodiments, the at least one perturbagen selected from Table 4, or a variant thereof, comprises at least 2, at least 3, at least 4, at least 5, at least 6, at least 7, at least 8, at least 9, at least 10, at least 11, at least 12, or 13 perturbagens selected from Table 4, or variants thereof.

In some embodiments, the one or more genes selected from Table 3 comprises 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more, or 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, 67 or more, or 68 or more genes designated as a “down” gene in the gene directionality column of Table 3.

In some embodiments, the one or more genes designated as a “down” gene in the gene directionality column of Table 3 are selected from PLP2, GAPDH, SNCA, CDH3, FKBP4, CAMSAP2, PPP1R13B, NISCH, HTRA1, ATP11B, ETS1, CPSF4, TLE1, CDK2, SESN1, GRB7, CERK, ZNF318, MYC, ELOVL6, STAMBP, EBNA1BP2, MSH6, FAH, EIF4EBP1, SLC35F2, RRP1B, G3BP1, UTP14A, DUSP3, FHL2, VPS72, ARL4C, ARPP19, CDKN1B, TP53, CRYZ, PLOD3, DDIT4, LAMA3, INPP1, CDK7, KLHL21, TIAM1, TIPARP, FOXJ3, NPC1, TUBB6, TPM1, RPA3, SFN, ST3GAL5, GMNN, ACOT9, BLMH, NNT, USP1, FKBP14, HSPB1, TBP, EPB41L2, CDCA4, TRAM2, CETN3, ETRN, PDLIM1, BRCA1, and LOXL1.

In some embodiments, the change in cells state provides one or more of: (a) decreased secretion of mucus by a goblet cell and (b) decreased synthesis of one or more mucins, optionally selected from MUCSAC, MUCSB, MUC2, MUC4, MUC7, MUC8, and MUC19.

In some embodiments, contacting the population of progenitor cells occurs in vitro or ex vivo or in vivo in a subject.

In certain aspects, the present disclosure is related to a perturbagen for use in a method of the present disclosure.

In certain aspects, the present disclosure is related to a pharmaceutical composition comprising a perturbagen of the present disclosure.

In certain aspects, the present disclosure is related to a method for promoting the formation of a goblet cell, or an immediate progenitor thereof, comprising: exposing a starting population of intestinal stem cells to a perturbation having a perturbation signature that promotes the transition of the starting population of intestinal stem cells into a M₀ cell or a goblet cell, wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1.

In some embodiments, the perturbation signature comprises an increase in expression and/or activity of one or more genes in the progenitor cell of an activation of a network module designated in the network module column of Table 1.

In certain aspects, the present disclosure is related to a method for inhibiting the formation of a goblet cell and/or a ciliated cell, or an immediate progenitor thereof, comprising: exposing a starting population of progenitor cells comprising at least one basal cell to a perturbation having a perturbation signature that prevents progression of a progenitor cell into and/or reduces the likelihood that a progenitor cell will progress into a goblet cell or other lineage associated progenitor thereof, wherein the perturbation signature comprises a decreased expression and/or activity in the progenitor cells of one or more genes selected from Table 3 designated as a “down” gene in the gene directionality column of Table 3.

In some embodiments, the formation of goblet cells is inhibited.

In some embodiments, the perturbation signature comprises an activation of a network module designated in the network module column of Table 3.

In certain aspects, the present disclosure is related to a method for treating a disease or disorder characterized by an abnormal number or function of goblet cells, comprising: (a) administering to a patient in need thereof a therapeutically effective amount of at least one perturbagen selected from Table 2 and/or Table 4, or a variant thereof, including combinations of the foregoing, wherein the at least one perturbagen is capable of changing a gene signature in a progenitor cell or (b) administering to a patient in need thereof a cell, the cell having been contacted with at least one perturbagen selected from Table 2 and/or Table 4, or a variant thereof, including combinations of the foregoing, wherein the at least one perturbagen is capable of changing a gene signature in a progenitor cell.

In some embodiments, the disease or disorder is caused by a goblet cell deficiency.

In some embodiments, the disease or disorder is selected from intestinal infection, colitis, inflammatory bowel disease (IBD), ulcerative colitis, cystic fibrosis, and cancer.

In some embodiments, the disease or disorder is caused by an increase in the number of goblet cells or an increase in the production of mucus by goblet cells.

In some embodiments, the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

In certain aspects, the present disclosure is related to a method for treating a disease or disorder characterized by an abnormal ratio of goblet cells to intestinal stem cells and/or basal cells, comprising: (a) administering to a patient in need thereof at least one perturbagen selected from Table 2 and/or Table 4, or a variant thereof, including combinations of the foregoing, wherein the at least one perturbagen is capable of changing a gene signature in an intestinal stem cell and/or a basal cell, or (b) administering to a patient in need thereof a cell, the cell having been contacted with at least one perturbagen selected from Table 2 and/or Table 4, or a variant thereof, including combinations of the foregoing, wherein the at least one perturbagen is capable of changing a gene signature in an intestinal stem cell and/or a basal cell.

In some embodiments, the abnormal ratio comprises a decreased number of goblet cells and/or an increased number of intestinal stem cells.

In some embodiments, the disease or disorder is selected from intestinal infection, colitis, inflammatory bowel disease (IBD), ulcerative colitis, cystic fibrosis, and cancer.

In some embodiments, the at least one perturbagen is capable of changing a gene signature in a progenitor cell.

In some embodiments, the abnormal ratio comprises a decreased number of goblet cells and/or an increased number of basal cells and/or an increased number of basal cells and/or a decreased number of goblet cells.

In some embodiments, the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

In some embodiments, the at least one perturbagen is capable of changing a gene signature in a basal cell.

In some embodiments, the patient is selected by steps comprising: obtaining from the patient having the disease or disorder a sample of cells comprising at least one intestinal stem cell and/or at least one basal cell; and contacting the sample of cells with least one perturbagen selected from Table 2 and/or Table 4, or a variant thereof, including combinations of the foregoing, wherein the at least one perturbagen alters a gene signature in the sample of cells.

In some embodiments, the patient is selected by steps comprising: obtaining from a subject having the disease or disorder a sample of cells comprising at least one intestinal stem cell and/or at least one basal cell; and contacting the sample of cells with at least one perturbagen capable of altering a gene signature in an intestinal stem cell and/or a basal cell, wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, and/or decreases in the sample of cells the expression and/or activity of one or more genes selected from Table 3 designated as a “down” gene in the gene directionality column of Table 3.

In some embodiments, the patient is selected by steps comprising: obtaining from a subject having the disease or disorder a sample of cells comprising an intestinal stem cell and/or a basal cell; and contacting the sample of cells with at least one perturbagen selected from Table 2 and/or Table 4, or a variant thereof, including combinations of the foregoing; wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1 and/or decreases in the sample of cells the expression and/or activity of one or more genes selected from Table 3 designated as a “down” gene in the gene directionality column of Table 3.

In some embodiments, the perturbagen causes an increase in expression and/or activity of one or more genes in the progenitor cell of an activation of a network module designated in the network module column of Table 1 and/or the perturbation signature comprises an activation of a network module designated in the network module column of Table 3.

In certain aspects, the present disclosure is related to a method for selecting the patient of any one of the methods of the present disclosure, comprising: obtaining from a subject having the disease or disorder a sample of cells comprising an intestinal stem cell and/or a basal cell; and contacting the sample of cells with least one perturbagen selected from Table 2 and/or Table 4, or a variant thereof, including combinations of the foregoing, wherein when the at least one perturbagen alters a gene signature in the sample of cells, the subject is selected as a patient.

In certain aspects, the present disclosure is related to a method for selecting the patient of any one of the methods of the present disclosure, comprising: obtaining from a subject having the disease or disorder a sample of cells comprising an intestinal stem cell; and contacting the sample of cells with at least one perturbagen capable of altering a gene signature in an intestinal stem cell and/or a basal cell, wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, and/or decreases in the sample of cells the expression and/or activity of one or more genes selected from Table 3 designated as a “down” gene in the gene directionality column of Table 3, the subject is selected as a patient.

In some embodiments, altering the gene signature comprises a change in expression and/or activity of one or more genes in the progenitor cell of a network module designated in the network module column of Table 1 and/or the method alters a gene signature in the sample of cells, comprising activation of a network module designated in the network module column of Table 3.

In certain aspects, the present disclosure is related to a method for selecting the patient of any one of the methods of the present disclosure, comprising: obtaining from a subject having the disease or disorder a sample of cells comprising an intestinal stem cell and/or a basal cell; and contacting the sample of cells with at least one perturbagen selected from Table 2 and/or Table 4, or a variant thereof, including combinations of the foregoing; wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, and/or decreases in the sample of cells the expression and/or activity of one or more genes selected from Table 3 designated as a “down” gene in the gene directionality column of Table 3, the subject is selected as a patient.

In some embodiments, the perturbagen causes an increase in expression and/or activity of one or more genes in the progenitor cell of an activation of a network module designated in the network module column of Table 1 and/or the method alters a gene signature in the sample of cells, comprising activation of a network module designated in the network module column of Table 3.

In certain aspects, the present disclosure is related to use of the perturbagen of Table 2, or a variant thereof in the manufacture of a medicament for treating a disease or disorder characterized by an abnormal ratio of goblet cells to intestinal stem cells and/or an abnormal ratio of goblet cells to enterocytes, Paneth cells and/or enteroendocrine cells.

In certain aspects, the present disclosure is related to use of the perturbagen of Table 4, or a variant thereof in the manufacture of a medicament for treating a disease or disorder characterized by an abnormal ratio of goblet cells to basal cells and/or an abnormal ratio of goblet cells to basal luminal cells, club cells, ciliated cells, and/or neuroendocrine cells.

In certain aspects, the present disclosure is related to a method of identifying a candidate perturbation for promoting the transition of a starting population of intestinal stem cells and/or basal cells into goblet cells or immediate progenitors thereof, the method comprising: exposing the starting population of intestinal stem cells and/or basal cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of intestinal stem cells and/or basal cells into goblet cells or immediate progenitors thereof following exposure of the population of cells to the perturbation; and identifying the perturbation as a candidate perturbation for promoting the transition of a population of intestinal stem cells and/or basal cells into goblet cells or immediate progenitors thereof based on the perturbation signature, wherein the perturbation signature is an increase in expression and/or activity in the intestinal stem cell of one or more genes designated as an “up” gene in the gene directionality column of Table 1, and/or a decrease in expression and/or activity in the intestinal stem cell of one or more genes designated as a “down” gene in the gene directionality column of Table 1, and/or a decrease in expression and/or activity in the basal cell of one or more genes selected from Table 3 designated as a “down” gene in the gene directionality column of Table 3.

In some embodiments, the perturbation signature is an increase in expression and/or activity in the progenitor cell of an activation of a network module designated in the network module column of Table 1 and/or an activation of a network module designated in the network module column of Table 3.

In certain aspects, the present disclosure is related to a method for making a therapeutic agent for a disease or disorder selected from intestinal infection, colitis, inflammatory bowel disease (IBD), ulcerative colitis, cystic fibrosis, and cancer, comprising: (a) identifying a candidate perturbation according to a method of the present disclosure and (b) formulating the candidate perturbation as a therapeutic agent for the treatment of the disease or disorder.

In certain aspects, the present disclosure is related to a method for making a therapeutic agent for a disease or disorder characterized by an abnormal ratio of goblet cells to basal luminal cells, club cells, ciliated cells, and/or neuroendocrine cells, comprising: (a) identifying a candidate perturbation according to a method of the present disclosure; and (b) formulating the candidate perturbation as a therapeutic agent for the treatment of the disease or disorder.

In certain aspects, the present disclosure is related to a method for inhibiting a change in cell state of a progenitor cell, comprising: contacting a population of cells comprising a progenitor cell with at least one perturbagen capable of altering a gene signature in the progenitor cell, wherein altering the gene signature comprises a decrease in expression and/or activity in the progenitor cell of one or more genes designated as a “down” gene in the gene directionality column of Table 3; wherein the progenitor cell is a basal cell; and wherein inhibiting the change in cell state provides a decrease in the number of goblet cells; an increase in the number of club cells; an increase in the number of ciliated cells, and wherein the change in cells state provides one or more of: (a) decreased secretion of mucus by a goblet cell and (b) decreased synthesis of one or more mucins, optionally selected from MUC5AC, MUC5B, MUC2, MUC4, MUC7, MUC8, and MUC19.

In some embodiments, the decrease in the number of goblet cells is relative to the number of basal cells or basal luminal precursor cells or club cells or ciliated cells or neuroendocrine cells obtained from a population of progenitor cells i) that is not contacted with the at least one perturbagen or ii) prior to contacting with the at least one perturbagen.

In some embodiments, the increase in the number of club cells and/or ciliated cells is relative to the number of basal cells, goblet cells, basal luminal precursor cells, ciliated cells, and/or neuroendocrine cells obtained from a population of progenitor cells i) that is not contacted with the at least one perturbagen or ii) prior to contacting with the at least one perturbagen.

In some embodiments, the at least one perturbagen is selected from Table 4, or a variant thereof.

In some embodiments, the at least one perturbagen selected from Table 4, or a variant thereof, comprises at least 2, at least 3, at least 4, at least 5, at least 6, at least 7, at least 8, at least 9, at least 10, at least 11, at least 12, or 13 perturbagens selected from Table 4, or variants thereof.

In some embodiments, the one or more genes designated as a “down” gene in the gene directionality column of Table 3 are selected from PLP2, GAPDH, SNCA, CDH3, FKBP4, CAMSAP2, PPP1R13B, NISCH, HTRA1, ATP11B, ETS1, CPSF4, TLE1, CDK2, SESN1, GRB7, CERK, ZNF318, MYC, ELOVL6, STAMBP, EBNA1BP2, MSH6, FAH, EIF4EBP1, SLC35F2, RRP1B, G3BP1, UTP14A, DUSP3, FHL2, VPS72, ARL4C, ARPP19, CDKN1B, TP53, CRYZ, PLOD3, DDIT4, LAMA3, INPP1, CDK7, KLHL21, TIAM1, TIPARP, FOXJ3, NPC1, TUBB6, TPM1, RPA3, SFN, ST3GAL5, GMNN, ACOT9, BLMH, NNT, USP1, FKBP14, HSPB1, TBP, EPB41L2, CDCA4, TRAM2, CETN3, ETRN, PDLIM1, BRCA1, and LOXL1.

Any aspect or embodiment disclosed herein can be combined with any other aspect or embodiment as disclosed herein.

### BRIEF DESCRIPTION OF THE DRAWINGS

FIG. 1 shows results obtained upon treatment of mouse intestinal organoids with the indicated compounds. DAPT alone or combination of DAPT and IWP2 induces the expression of muc2 gene, a marker of Goblet cells.

FIG. 2 shows that mice treated with peturbagens targeting goblet cells attenuate total clinical score to DSS. * p<0.05; ** p<0.005.

FIGS. 3A-3B show that goblet hyperplasia is a driver of disease pathology in muco-obstructive diseases such as chronic obstructive pulmonary disease (COPD). FIG. 3A shows images of goblet cells and ciliated cells in a healthy lung and a COPD lung (Gohy et al., Sci Rep. 9:17963 (2019), which is incorporated by reference herein in its entirety).

FIG. 3B shows a schematic of a mechanism depicting goblet hyperplasia and a mechanism depicting inhibition of goblet cell hyperplasia and restored cellular homeostasis (Gkatzis et al., Eur Respir J 52(5):1800876 (2018), which is incorporated by reference herein in its entirety).

FIGS. 4A-4B show images demonstrating that scRNA-seq datasets of asthma patients and an in vitro model of goblet hyperplasia are used with machine learning to predict compounds to inhibit goblet cell differentiation. FIG. 4A shows an in vitro air-liquid interface model of the airway epithelium, human bronchial epithelial cells (HBECs) from a healthy donor differentiated for 8 days in the absence of IL-13. FIG. 4B shows an in vitro air-liquid interface model of the airway epithelium, human bronchial epithelial cells (HBECs) from a healthy donor differentiated for 8 days in the presence of IL-13 and sequenced at a single cell level.

FIG. 5 shows an image of an IL-13 induced air-liquid interface (ALI) model of goblet cell hyperplasia (adapted from www.stemcell.com).

FIGS. 6A-6C show experimental data demonstrating that three compounds inhibited goblet hyperplasia and maintained healthy cellular populations in COPD donor HBECs treated with IL-13 to induce goblet hyperplasia. The HBECs included MUC5Ac (goblet cells) (FIG. 6A), FOXJ1 (ciliated cells) (FIG. 6B), and SCGB1A1 (club cells) (FIG. 6C). One-way ANOVA, Dunnett's multiple comparisons test on transformed data *p<0.01, **p<0.01, ***p<0.001, ****p<0.0001, compared to DMSO; 3 donors; 2 transwells per donor.

FIGS. 7A-7B show that histological analysis confirms inhibition of goblet hyperplasia and restoration of ciliated cell populations. FIG. 7A shows immunohistochemistry (IHC) quantification of the % MUC5AC positive area normalized to DMSO for three compounds. FIG. 7B shows representative histology images of COPD HBEC donor cells.

FIGS. 8A-8B show experimental data demonstrating that three compounds inhibit Muc5ac in additional COPD relevant goblet hyperplasia models. FIG. 8A shows orthogonal assays of COPD patient small airway cells (SAECs)+IL-13 cell cultures processed for qPCR and evaluated for Muc5ac. FIG. 8B shows orthogonal assays of COPD human bronchial epithelial cells (HBECs)+IL-17 cell cultures processed for qPCR and evaluated for Muc5ac.

FIG. 9 shows experimental data demonstrating in vivo efficacy of compound A, which significantly inhibits Muc5ac in an intranasal IL-13 mouse model of goblet hyperplasia.

FIG. 10A. illustrates an experimental scheme of culturing the undifferentiated basal cells known as primary human bronchial epithelial cells (HBECs) at the air-liquid interface in the presence or absence of IL-13 and the perturbagens (See Examples 1 and 2 infra).

FIG. 10B provides micrographs that illustrate the testing results for Air-liquid interface (ALI) differentiation of HBECs in the presence or absence of IL-3. Pseudostratified airway epithelium was formed by day 14 of ALI differentiation in the presence of 1 ng/mL of IL-3. Total mucus (AB/PAS), goblet cells (Muc5ac⁺), and ciliated cells (acetyl α-tubulin⁺) were visualized via immunofluorescence or histology. For histology analysis, the differentiated HBECs on day 14 of ALI were stained with AB/PAS, Muc5ac, and acetyl α-tubulin. For Immunofluorescence imaging, the differentiated HBECs on day 14 of ALI were stained with DAPI, Muc5ac, and acetyl α-tubulin. Immunofluorescence allowed for a top down view of the most apical layer of cells, while histology presented a cross-section of the pseudostratified epithelium and was amenable for image quantification (See Example 5 infra).

FIG. 11A-FIG. 11B illustrate that active compounds were effective in blocking goblet hyperplasia and restored ciliated cells. FIG. 11A illustrates that HBECs from healthy donors were differentiated at air-liquid interface (ALI) in the presence or absence of 1 ng/ml IL-13 and treated with predicted compounds. Anti-Notch2 neutralizing antibody was used as a positive control. On day 14 of ALI, samples were fixed, processed for histology, anti-Muc5ac (goblet cells) and anti-acetyl α-Tubulin (ciliated cells) antibodies. FIG. 11B is a box plot that illustrates that Muc5ac % positive stained area was quantified on histology images.

FIG. 12A-FIG. 12E illustrate that perturbagen analogs were effective in blocking goblet hyperplasia. FIG. 12A and FIG. 12B are box plots that illustrate that analogs and Perturbagen 3 related molecules block goblet hyperplasia. FIG. 12C and FIG. 12D are box plots that illustrate that analogs and Perturbagen 3 related molecules maintain or increase ciliated cells. HBECs of healthy and diseased (asthma, COPD) donors were differentiated at ALI in the presence of IL-13 (0.3 ng/ml diseased donors, 1 ng/ml healthy donors). At day 14 of differentiation cultures were processed for histology and stained for Muc5ac (FIG. 12A and FIG. 12B) and acetyl α-Tubulin (FIG. 12C and FIG. 12D). To assess % stained area, 10 IHC images per condition were quantified. Analogs and Perturbagen 3 related molecules compounds blocked goblet hyperplasia and maintained or increased ciliated cells in healthy donors; Perturbagen 6 also blocked goblet hyperplasia and maintained ciliated cells in diseased donors. FIG. 12E is a plot that illustrates that Perturbagen 6<Perturbagen 3<Perturbagen 4 (IC50) and Perturbagen 6 as an analog of Perturbagen 3 with >10-fold enhanced potency (picomolar activity) in decreasing goblet cell mucus production. To compare IC50 of selected analogs, HBECs from two healthy donors were differentiated in ALI in the presence of 1 ng/ml IL-13 and a range of 10 concentrations of Perturbagen 3, Perturbagen 4, and Perturbagen 6. At day 14, cultures were lysed, RNA extracted, and cDNA generated. Goblet hyperplasia was assessed by Muc5ac qPCR. For IC50 calculation, 2\hat{ }-ΔCt values were transformed to log form and normalized. Curve fit was calculated using non-linear regression and Log IC50 of compounds were statistically compared via extra sum of squares F test.

### DETAILED DESCRIPTION

The present disclosure is based, in part, on the discovery that cells of intestinal lineages comprising goblet progenitors, Paneth cells, enteroendocrine cells, and goblets cells and their progenitors can be characterized by specific gene signatures, and on the discovery that goblet cells, ciliated cells and their progenitors can be characterized by specific gene signatures. Additionally, the present disclosure is based on the discovery that certain active agents (i.e., perturbagens) can alter these specific gene signatures. Additionally, the present disclosure is based on the discovery that certain active agents (i.e., perturbagens) can alter these specific gene signatures. Such alteration is associated with the acquisition of specific cell states by cells of goblet lineages and/or secretory cell lineages. These perturbagens are, in some instance, useful as therapeutics and derive benefit by directing the intestinal stem cells towards M₀ and/or goblet cells and/or by directing the progenitor cells away from goblet cell states, and are thereby are useful in the methods of treatment of chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma, or related diseases or disorders described infra, in subjects in need thereof.

In aspects, the disclosure also provides a method for treating a disease or disorder characterized by an abnormal number or function of goblet cells. In embodiments, the method comprises: a) administering to a patient in need thereof a therapeutically effective amount of at least one perturbagen selected from Table 2 and/or Table 4, or a variant thereof, including combinations of the foregoing, wherein the at least one perturbagen is capable of changing a gene signature in a progenitor cell or (b) administering to a patient in need thereof a cell, the cell having been contacted with at least one perturbagen selected from Table 2 and/or Table 4, or a variant thereof, including combinations of the foregoing, wherein the at least one perturbagen is capable of changing a gene signature in a progenitor cell. In embodiments, the method comprises: (a) administering to a patient in need thereof at least one perturbagen selected from Table 2 and/or Table 4, or a variant thereof, including combinations of the foregoing, wherein the at least one perturbagen is capable of changing a gene signature in an intestinal stem cell and/or a basal cell, or (b) administering to a patient in need thereof a cell, the cell having been contacted with at least one perturbagen selected from Table 2 and/or Table 4, or a variant thereof, including combinations of the foregoing, wherein the at least one perturbagen is capable of changing a gene signature in an intestinal stem cell and/or a basal cell. In embodiments, the method comprises: a) administering to a patient in need thereof a therapeutically effective amount of at least one perturbagen selected from Table 2 and/or Table 4, or a variant thereof, including combinations of the foregoing, wherein the at least one perturbagen is capable of changing one or more gene signatures in a plurality of progenitor cells or (b) administering to a patient in need thereof a cell or a plurality of cells, the cell and/or plurality of cells having been contacted with at least one perturbagen selected from Table 2 and/or Table 4, or a variant thereof, including combinations of the foregoing, wherein the at least one perturbagen is capable of changing one or more gene signatures in a plurality of progenitor cells. In embodiments, the method comprises: (a) administering to a patient in need thereof at least one perturbagen selected from Table 2 and/or Table 4, or a variant thereof, including combinations of the foregoing, wherein the at least one perturbagen is capable of changing one or more gene signatures in a plurality of intestinal stem cells and/or a plurality of basal cells, or (b) administering to a patient in need thereof a cell or a plurality of cells, the cell and/or plurality of cells having been contacted with at least one perturbagen selected from Table 2 and/or Table 4, or a variant thereof, including combinations of the foregoing, wherein the at least one perturbagen is capable of changing one ore more gene signatures in a plurality of intestinal stem cells and/or a plurality of basal cells. In embodiments, the method provides the combinatorial use of perturbagens and/or gene signatures to simultaneously drive and/or direct a change in the number or function of goblet cells (e.g. an increase in the number of goblet cells, a decrease in the number of goblet cells, and/or an increase in the production of mucus by goblet cells) useful for methods including but not limited to disease or disorder characterized by an abnormal number or function of goblet cells. In a non-limiting example, the method includes contacting a plurality of progenitor cells with at least one perturbagen (e.g. at least about one, about 2, about 3, about 4, about 5, about 6, about 7, about 8, about 9, about 10, or about 10 or more perturbagens) selected from Table 2, and/or Table 4, or a variant thereof, including combinations of the foregoing (e.g. a combination of perturbagens) wherein the at least one perturbagen (e.g. combination of perturbagens) provides alteration of one or more gene signatures, for example one or more gene signatures (e.g. about 2 or more, about 3 or more, about 4 or more, about 5 or more, about 6 or more, about 7 or more, about 8 or more, about 9 or more, or about 10 or more gene signatures) selected from Table 1 and/or Table 3, including combinations of the foregoing, and any combination thereof. In embodiments, the alteration of one or more gene signatures provides an increase in the number of one or more of goblet progenitors, goblet cells, Paneth cells, and enteroendocrine cells. In embodiments, the alteration of one or more gene signatures provides a decrease in the number of goblet cells; a decrease in the number of ciliated cells, an increase in the number of club cells. As would be understood by one of ordinary skill in the art, any of the perturbagens and/or methods and/or compositions described herein can be used for changing one or more gene signatures of a plurality of progenitor cells (e.g. a plurality of intestinal stem cells, a plurality of basal cells), including but not limited to use in methods for selecting a patient of any of the methods described herein.

### Goblet Cells

### Genes Signatures

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins that are characteristic of the cell type or cells of a specific lineage.

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

Genes that are differentially expressed and positively associated with the promotion of goblet cells lineage progression and/or M₀ cell differentiation are listed in Table 1.

TABLE 1

| | Gene | Gene_Entrez_ID | Gene_Directionality | Network_Module |
|---|---|---|---|---|
| 0 | BIRC5 | 332 | up | 2 |
| 1 | CCNB1 | 891 | up | 2 |
| 2 | UBE2C | 11065 | up | 2 |
| 3 | CDC20 | 991 | up | 2 |
| 4 | CCNA2 | 890 | up | 2 |
| 5 | MIF | 4282 | down | 2 |
| 6 | TRAP1 | 7190 | down | 2 |
| 7 | CETN3 | 1070 | down | 2 |
| 8 | CHEK2 | 11200 | down | 2 |
| 9 | RAE1 | 8480 | down | 2 |
| 10 | S100A13 | 6284 | up | 3 |
| 11 | HES1 | 3280 | down | 3 |
| 12 | HADH | 3033 | down | 3 |
| 13 | RFC2 | 5982 | down | 3 |
| 14 | MCM3 | 4172 | down | 3 |
| 15 | PCNA | 5111 | down | 3 |
| 16 | HAT1 | 8520 | down | 3 |
| 17 | TOPBP1 | 11073 | down | 3 |
| 18 | EED | 8726 | down | 3 |
| 19 | DDB2 | 1643 | down | 3 |
| 20 | PMM2 | 5373 | up | 4 |
| 21 | GADD45B | 4616 | up | 4 |
| 22 | GADD45A | 1647 | up | 4 |
| 23 | PKIG | 11142 | down | 4 |
| 24 | SNX7 | 51375 | down | 4 |
| 25 | DYRK3 | 8444 | down | 4 |
| 26 | CCNH | 902 | down | 4 |
| 27 | PHGDH | 26227 | down | 4 |
| 28 | RAP1GAP | 5909 | up | 5 |
| 29 | TM9SF2 | 9375 | up | 5 |
| 30 | TMED10 | 10972 | up | 5 |
| 31 | STXBP1 | 6812 | up | 5 |
| 32 | GLRX | 2745 | down | 5 |
| 33 | CHP1 | 11261 | down | 5 |
| 34 | NR3C1 | 2908 | down | 5 |
| 35 | TSC22D3 | 1831 | down | 5 |
| 36 | GALE | 2582 | up | 6 |
| 37 | KDELR2 | 11014 | up | 6 |
| 38 | ADGRE5 | 976 | up | 6 |
| 39 | MTHFD2 | 10797 | up | 6 |
| 40 | FHL2 | 2274 | up | 6 |
| 41 | PYCR1 | 5831 | up | 6 |
| 42 | EIF5 | 1983 | down | 6 |
| 43 | FOXO3 | 2309 | down | 6 |
| 44 | MVP | 9961 | up | 7 |
| 45 | SOX4 | 6659 | down | 7 |
| 46 | H2AFV | 94239 | down | 7 |
| 47 | GRB7 | 2886 | down | 7 |
| 48 | SNX6 | 58533 | down | 7 |
| 49 | NOTCH1 | 4851 | down | 7 |
| 50 | NET1 | 9152 | down | 7 |
| 51 | BAMBI | 25805 | up | 8 |
| 52 | CD44 | 960 | down | 8 |
| 53 | VDAC1 | 7416 | down | 8 |
| 54 | CREG1 | 8804 | down | 8 |
| 55 | PRSS23 | 11098 | down | 8 |
| 56 | FKBP4 | 2288 | down | 8 |
| 57 | MBNL1 | 4154 | down | 8 |
| 58 | BAD | 572 | up | 9 |
| 59 | HMGCS1 | 3157 | up | 9 |
| 60 | ATP6VOB | 533 | up | 9 |
| 61 | FDFT1 | 2222 | up | 9 |
| 62 | HMGCR | 3156 | up | 9 |
| 63 | EBP | 10682 | up | 9 |
| 64 | ACLY | 47 | up | 9 |
| 65 | MYC | 4609 | down | 10 |
| 66 | MRPL12 | 6182 | down | 10 |
| 67 | CD320 | 51293 | down | 10 |
| 68 | G3BP1 | 10146 | down | 10 |
| 69 | CBR1 | 873 | down | 10 |
| 70 | IFRD2 | 7866 | down | 10 |
| 71 | GRWD1 | 83743 | down | 10 |
| 72 | FGFR2 | 2263 | up | 11 |
| 73 | STAP2 | 55620 | up | 11 |
| 74 | RPS5 | 6193 | down | 11 |
| 75 | TSPAN3 | 10099 | down | 11 |
| 76 | ADCK3 | 56997 | down | 11 |
| 77 | OXCT1 | 5019 | down | 11 |
| 78 | GPER1 | 2852 | down | 11 |
| 79 | XBP1 | 7494 | up | 13 |
| 80 | GFPT1 | 2673 | up | 13 |
| 81 | CDKN1A | 1026 | up | 13 |
| 82 | HYOU1 | 10525 | up | 13 |
| 83 | ACBD3 | 64746 | up | 13 |
| 84 | PDGFA | 5154 | down | 13 |
| 85 | COPB2 | 9276 | up | 14 |
| 86 | HERPUD1 | 9709 | up | 14 |
| 87 | NUCB2 | 4925 | up | 14 |
| 88 | TRIM2 | 23321 | down | 14 |
| 89 | APP | 351 | down | 14 |
| 90 | UGDH | 7358 | down | 14 |
| 91 | CDC25B | 994 | up | 15 |
| 92 | PSME1 | 5720 | down | 15 |
| 93 | MTA1 | 9112 | down | 15 |
| 94 | RPA2 | 6118 | down | 15 |
| 95 | PGRMC1 | 10857 | down | 15 |
| 96 | PSIP1 | 11168 | down | 15 |
| 97 | BACE2 | 25825 | up | 16 |
| 98 | RGS2 | 5997 | up | 16 |
| 99 | HSPD1 | 3329 | down | 16 |
| 100 | PAICS | 10606 | down | 16 |
| 101 | SDHB | 6390 | down | 16 |
| 102 | NOLC1 | 9221 | down | 16 |
| 103 | RPN1 | 6184 | up | 17 |
| 104 | RAB27A | 5873 | up | 17 |
| 105 | SLC35A1 | 10559 | up | 17 |
| 106 | ATP1B1 | 481 | down | 17 |
| 107 | PLA2G4A | 5321 | down | 17 |
| 108 | TOMM70A | 9868 | down | 17 |
| 109 | RNH1 | 6050 | up | 18 |
| 110 | C2CD2L | 9854 | up | 18 |
| 111 | ARHGAP1 | 392 | up | 18 |
| 112 | ADH5 | 128 | down | 18 |
| 113 | MRPL19 | 9801 | down | 18 |
| 114 | SLC35B1 | 10237 | up | 19 |
| 115 | CRELD2 | 79174 | up | 19 |
| 116 | UFM1 | 51569 | up | 19 |
| 117 | ARFIP2 | 23647 | up | 19 |
| 118 | AARS | 16 | down | 19 |
| 119 | KIT | 3815 | up | 20 |
| 120 | FAIM | 55179 | up | 20 |
| 121 | SOCS2 | 8835 | down | 20 |
| 122 | HEBP1 | 50865 | down | 20 |
| 123 | TIMM9 | 26520 | down | 20 |
| 124 | NPDC1 | 56654 | up | 21 |
| 125 | NIPSNAP1 | 8508 | down | 21 |
| 126 | OXA1L | 5018 | down | 21 |
| 127 | MSRA | 4482 | down | 21 |
| 128 | ARID5B | 84159 | down | 22 |
| 129 | BZW2 | 28969 | down | 22 |
| 130 | EDN1 | 1906 | down | 22 |
| 131 | POLR1C | 9533 | down | 22 |
| 132 | TIMM17B | 10245 | up | 23 |
| 133 | CDK4 | 1019 | down | 23 |
| 134 | DCTD | 1635 | down | 23 |
| 135 | HSD17B10 | 3028 | down | 23 |
| 136 | PDIA5 | 10954 | up | 25 |
| 137 | TM9SF3 | 56889 | up | 25 |
| 138 | EDEM1 | 9695 | up | 25 |

The genes listed in Table 1 and classified as “up” in the gene directionality column of Table 1 show an increase in expression in the cell state change. The genes listed in Table 1 and classified as “down” in the gene directionality column of Table 1 show a decrease in expression in the cell state change.

In Table 1 and associated embodiments:
“Gene ID”: at the time of filing the present disclosure, the World Wide Web at ncbi.nlm.nih.gov/gene provides a description of and the nucleic acid sequence for each GeneID listed in Table 1; the contents of each of which is incorporated herein by reference in its entirety.
“Up” indicates a gene for which an increase in expression and/or activity in the progenitor cell is associated with the gene signature.
“Down” indicates a gene for which a decrease in expression and/or activity in the progenitor cell is associated with the gene signature.
A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition—such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 1, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 1. In certain embodiments, a perturbation activates multiple network modules for the desired cell state transition, such as 2, 3, 4, 5, 6, 7, 8, 9, 10, 11, 12, 13, 14, 15, 16, 17, 18, 19, 20, 21, 22, 23, 24, or more modules.

In some embodiments, one or more genes of network module 2 are modulated. In some embodiments, the present disclosure relates to the activation of network module 2, e.g., one or more of (inclusive of all of) BIRC5, CCNB1, UBE2C, CDC20, CCNA2, MIF, TRAP1, CETN3, CHEK2, and RAE1.

In some embodiments, one or more genes of network module 3 are modulated. In some embodiments, the present disclosure relates to the activation of network module 3, e.g., one or more of (inclusive of all of) S100A13, HES1, HADH, RFC2, MCM3, PCNA, HAT1, TOPBP1, EED, and DDB2.

In some embodiments, one or more genes of network module 4 are modulated. In some embodiments, the present disclosure relates to the activation of network module 4, e.g., one or more of (inclusive of all of) PMM2, GADD45B, GADD45A, PKIG, SNX7, DYRK3, CCNH, and PHGDH.

In some embodiments, one or more genes of network module 5 are modulated. In some embodiments, the present disclosure relates to the activation of network module 5, e.g., one or more of (inclusive of all of) RAP1GAP, TM9SF2, TMED10, STXBP1, GLRX, CHP1, NR3C1, and TSC22D3.

In some embodiments, one or more genes of network module 6 are modulated. In some embodiments, the present disclosure relates to the activation of network module 6, e.g., one or more of (inclusive of all of) GALE, KDELR2, ADGRE5, MTHFD2, FHL2, PYCR1, EIF5, and FOXO3.

In some embodiments, present disclosure relates to one or more genes of network module 7 are modulated. In some embodiments, the present disclosure relates to the activation of network module 7, e.g., one or more of (inclusive of all of) MVP, SOX4, H2AFV, GRB7, SNX6, NOTCH1, and NET1.

In some embodiments, one or more genes of network module 8 are modulated. In some embodiments, the present disclosure relates to the activation of network module 8, e.g., one or more of (inclusive of all of) BAMBI, CD44, VDAC1, CREG1, PRSS23, FKBP4, and MBNL1.

In some embodiments, one or more genes of network module 9 are modulated. In some embodiments, the present disclosure relates to the activation of network module 9, e.g., one or more of (inclusive of all of) BAD, HMGCS1, ATP6V0B, FDFT1, HMGCR, EBP, and ACLY.

In some embodiments, one or more genes of network module 10 are modulated. In some embodiments, the present disclosure relates to the activation of network module 10, e.g., one or more of (inclusive of all of) MYC, MRPL12, CD320, G3BP1, CBR1, IFRD2, and GRWD1.

In some embodiments, one or more genes of network module 11 are modulated. In some embodiments, the present disclosure relates to the activation of network module 11, e.g., one or more of (inclusive of all of) FGFR2, STAP2, RPS5, TSPAN3, ADCK3, OXCT1, and GPER1.

In some embodiments, one or more genes of network module 13 are modulated. In some embodiments, the present disclosure relates to the activation of network module 13, e.g., one or more of (inclusive of all of) XBP1, GFPT1, CDKN1A, HYOU1, ACBD3, and PDGFA.

In some embodiments, one or more genes of network module 14 are modulated. In some embodiments, the present disclosure relates to the activation of network module 14, e.g., one or more of (inclusive of all of) COPB2, HERPUD1, NUCB2, TRIM2, APP, and UGDH.

In some embodiments, one or more genes of network module 15 are modulated. In some embodiments, the present disclosure relates to the activation of network module 15, e.g., one or more of (inclusive of all of) CDC25B, PSME1, MTA1, RPA2, PGRMC1, and PSIP1.

In some embodiments, one or more genes of network module 16 are modulated. In some embodiments, the present disclosure relates to the activation of network module 16, e.g., one or more of (inclusive of all of) BACE2, RGS2, HSPD1, PAICS, SDHB, and NOLC1.

In some embodiments, one or more genes of network module 17 are modulated. In some embodiments, the present disclosure relates to the activation of network module 17, e.g., one or more of (inclusive of all of) RPN1, RAB27A, SLC35A1, ATP1B1, PLA2G4A, and TOMM70A.

In some embodiments, one or more genes of network module 18 are modulated. In some embodiments, the present disclosure relates to the activation of network module 18, e.g., one or more of (inclusive of all of) RNH1, C2CD2L, ARHGAP1, ADH5, and MRPL19.

In some embodiments, one or more genes of network module 19 are modulated. In some embodiments, the present disclosure relates to the activation of network module 19, e.g., one or more of (inclusive of all of) SLC35B1, CRELD2, UFM1, ARFIP2, and AARS.

In some embodiments, one or more genes of network module 20 are modulated. In some embodiments, the present disclosure relates to the activation of network module 20, e.g., one or more of (inclusive of all of) KIT, FAIM, SOCS2, HEBP1, and TIMM9.

In some embodiments, one or more genes of network module 21 are modulated. In some embodiments, the present disclosure relates to the activation of network module 21, e.g., one or more of (inclusive of all of) NPDC1, NIPSNAP1, OXA1L, and MSRA.

In some embodiments, one or more genes of network module 22 are modulated. In some embodiments, the present disclosure relates to the activation of network module 22, e.g., one or more of (inclusive of all of) ARID5B, BZW2, EDN1, and POLR1C.

In some embodiments, one or more genes of network module 23 are modulated. In some embodiments, the present disclosure relates to the activation of network module 23, e.g., one or more of (inclusive of all of) TIMM17B, CDK4, DCTD, and HSD17B10.

In some embodiments, one or more genes of network module 25 are modulated. In some embodiments, the present disclosure relates to the activation of network module 25, e.g., one or more of (inclusive of all of) PDIA5, TM9SF3, and EDEM1.

In some embodiments, the present methods alter a gene signature in the sample of cells, comprising an activation of a network module designated in the network module column of Table 1.

In some embodiments, the activation of the network module designated in the network module column of Table 1 comprises modulating expression and/or activity of 2 or more genes within a network module.

In some embodiments, the activation of the network module designated in the network module column of Table 1 comprises modulating expression and/or activity of all of the genes within a network module.

In some embodiments, the activation of the network module designated in the network module column of Table 1 comprises modulating expression and/or activity of 2 or more genes within 2 or more network modules. In some embodiments, the activation of the network module designated in the network module column of Table 1 comprises modulating expression and/or activity of 2 or more genes (e.g., 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more genes, 51 or more, 52 or more, 53 or more, 54 or more, 55 or more, 56 or more, 57 or more, 58 or more, 59 or more, 60 or more, 61 or more, 62 or more, 63 or more, 64 or more, 65 or ore, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or ore, 95 or more, 96 or more, 97 or more, 98 or more, 99 or more, 100 or more, 101 or more, 102 or more, 103 or more, 104 or more, 105 or more, 106 or more, 107 or more, 108 or more, 109 or more, 110 or more, 111 or more, 112 or more, 113 or more, 114 or more, 115 or more, 116 or more, 117 or more, 118 or more, 119 or more, 120 or more, 121 or ore, 122 or more, 123 or more, 124 or more, 125 or more, 126 or more, 127 or more, 128 or more, 129 or more, 130 or more, 131 or more, 132 or more, 133 or more, 134 or more, 135 or more, 136 or more, 137 or more, 138 or more, or 139 or more) within 2 or more network modules (e.g. 2 or more, or 3 or more, or 4 or more, or 5 or more, or 6 or more, or 7 or more, or 8 or more, or 9 or more, or 10 or more, 11 or more, 12 or more, 13 or more, 14 or more, 15 or more, 16 or more, 17 or more, 18 or more, 19 or more, 20 or more, 21 or more, 22 or more, 23 or more, or 24 or more network modules).

At the time of filing the present disclosure, the World Wide Web at ncbi.nlm.nih.gov/gene provides a description of and the nucleic acid sequence for each Gene designated as an “up” gene in the gene directionality column of Table 1; the contents of each of which is incorporated herein by reference in its entirety. At the time of filing the present disclosure, the World Wide Web at ncbi.nlm.nih.gov/gene provides a description of and the nucleic acid sequence for each Gene listed in the genes designated as a “down” gene in the gene directionality column of Table 1; the contents of each of which is incorporated herein by reference in its entirety.

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of promoting intestinal stem cells or progeny thereof are listed in Table 2.

TABLE 2

| Perturbagen | Molecular | Molecular | Dose | Dose |
|---|---|---|---|---|
| No. | formula | weight (g/mol) | In vitro | In vivo |
| 1 | C₁₈H₂₂N₂O₃S₂ | 378.5 | 0.1 μM, 1 μM | 10 mg/kg |
| 2 | C₂₃H₃₈N₂O₃ | 390.6 | 0.1 μM, 1 μM, | 10 mg/kg |
| | | | 10 μM | |
| 3 | C₁₂₄H₂₂N₂₂O₂₄ | 2405.2 | 0.01 μM, 0.1 μM, | 10 mg/kg |
| | | | 1 μM | |
| 4 | C₂₂H₂₅NO₆ | 399.4 | 0.1 μM, 1 μM, | 10 mg/kg |
| | | | 10 μM | |
| 5 | C₁₅H₁₅FIN₃O₃ | 431.2 | 0.1 μM, 1 μM, | 10 mg/kg |
| | | | 10 μM | |
| 6 | C₄₄H₄₇F₂N₉O₅S | 852.0 | 0.1 μM, 1 μM, | 10 mg/kg |
| | | | 10 μM | |
| 7 | C₂₀H₂₆N₂ | 294.4 | 0.1 μM, 1 μM, | 10 mg/kg |
| | | | 10 μM | |
| 8 | C₂₉H₃₇N₅O₃ | 503.6 | 0.1 μM, 0.2 μM, | 10 mg/kg |
| | | | 0.5 μM | |
| 9 | C₁₀H₁₃N₅O₄S | 299.3 | 0.1 μM, 0.5 μM, | 4.25 mg/kg |
| | | | 1 μM | |
| 10 | C₂₆H₄₃NO₅ | 449.6 | 0.1 μM, 1 μM, | 10 mg/kg |
| | | | 10 μM | |
| 11 | C₂₀H₂₄O₆ | 360.4 | 0.1 μM, 1 μM, | 10 mg/kg |
| | | | 10 μM | |
| 12 | C₂₇H₃₆N₆O₃S | 524.68 | | |
| 13 | C₃₀H₄₆O₃ | 454.7 | | |
| 14 | C₃₈H₇₂N₂O₁₂ | 748.98 | | |

In various embodiments herein, a perturbagen of Table 2 encompasses the perturbagens named in Table 2. Thus, the named perturbagens of Table 2 represent examples of perturbagens of the present disclosure.

In Table 2, the effective in vitro concentration is the concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In embodiments, a perturbagen used in the present disclosure is a variant of a perturbagen of Table 2. A variant may be a derivative, analog, enantiomer or a mixture of enantiomers thereof or a pharmaceutically acceptable salt, solvate, hydrate, co-crystal, clathrate, or polymorph of the perturbagen of Table 2. A variant of a perturbagen of Table 2 retains the biological activity of the perturbagen of Table 2.

### Methods and Perturbagens for Directing a Change in Cell State

Particular cellular changes in cell state can be matched to differential gene expression (which collectively define a gene signature), caused by exposure of a cell to a perturbagen. In embodiments, a change in cell state may be from one progenitor cell type to another progenitor cell type. In embodiments, a change in cell state may be from an upstream progenitor cell (e.g. early common progenitor) to a downstream progenitor cell. Lastly, in embodiments, a change in cell state may be from the final non-differentiated cell into a differentiated cell.

In some aspects, the present disclosure is related to a method for directing a change in cell state of a progenitor cell. This method includes a step of contacting a population of cells comprising a progenitor cell with at least one perturbagen selected from Table 2, or a variant thereof wherein the at least one perturbagen is capable of altering a gene signature in the progenitor cell; and wherein the progenitor cell is an intestinal stem cell. In some embodiments, the intestinal stem cell can be identified using a LGR2 marker (see Yin et al., Nat Methods. 2014 January; 11(1):106-12).

In aspects, the present disclosure is related to a method for directing a change in cell state of a progenitor cell, comprising contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In some aspects, the present disclosure is related to a method for directing a change in cell state of a progenitor cell, comprising contacting a population of cells comprising a progenitor cell with at least one perturbagen selected from Table 2, or a variant thereof, and capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In some embodiments, altering the gene signature comprises a change in expression and/or activity of one or more genes in the progenitor cell of a network module designated in the network module column of Table 1. In embodiments, the activation of one or more genes of the network module designated in the network module column of Table 1 comprises modulating expression and/or activity of 2 or more genes within a network module.

In some embodiments, the step of altering the gene signature comprises an increase in expression and/or activity in the progenitor cell of one or more genes designated as an “up” gene in the gene directionality column of Table 1. In other embodiments, the step of altering the gene signature comprises a decrease in expression and/or activity in the progenitor cell of one or more genes designated as a “down” gene in the gene directionality column of Table 1.

In some embodiments, the change in cell state provides an increase in the number of one or more of goblet progenitors, goblet cells, Paneth cells, and enteroendocrine cells. In other embodiments, the change in cell state provides an increase in the number of goblet cells.

For details on the goblet cell lineage, see, e.g., Mills, Jason C., and Jeffrey I. Gordon. “The intestinal stem cell niche: there grows the neighborhood.” Proceedings of the National Academy of Sciences 98.22 (2001): 12334-12336, the entire contents of which are incorporated by reference.

In some embodiments, the increase in the number of goblet cells is relative to the number of goblets cells obtained from a population of progenitor cells that is not contacted with the at least one perturbagen. In embodiments, the increase in the number of goblet cells is relative to the number of goblets cells obtained from a population of progenitor cells prior to contacting with the at least one perturbagen.

In some embodiments, the change in cell state does not provide a substantial increase in the number of enterocytes and/or provides a decrease in the number of enterocytes.

In some embodiments, the ratio of the number of goblet cells to the number of enterocytes is increased relative to the ratio obtained from a population of progenitor cells that is not contacted with the at least one perturbagen. In embodiments, the ratio of the number of goblet cells to the number of enterocytes is increased relative to the ratio in the population of progenitor cells prior to contacting with the at least one perturbagen. In embodiments, the ratio of the number of goblet cells to the number of progenitor cells is increased relative to the ratio obtained from a population of progenitor cells that is not contacted with the at least one perturbagen. In some embodiments, the ratio of the number of goblet cells to the number of progenitor cells is increased relative to the ratio in the population of progenitor cells prior to contacting with the at least one perturbagen.

In some embodiments, the methods described herein cause an increase in the number of goblet cells which, e.g., is due in part to increased cell proliferation of the goblet cells. In some embodiments, the increase in the number of goblet cells is due in part to an increased lifespan of the goblet cells. In other embodiments, the increase in the number of goblet cells is due in part to reduced cell death among the goblet cells.

In some embodiments, the methods described herein are such that the number of progenitor cells is decreased. In some embodiments, the decrease in the number of progenitor cells is due in part to decreased cell proliferation of the progenitor cells. In other embodiments, the decrease in the number of progenitor cells is due in part to a decreased lifespan of the progenitor cells. In embodiments, the decrease in the number of progenitor cells is due in part to increased cell death among the progenitor cells.

In some embodiments, the decrease in the number of progenitor cells is relative to the number of progenitor cells in a population of progenitor cells that is not contacted with the at least one perturbagen. In embodiments, the decrease in the number of progenitor cells is relative to the number of progenitor cells in the population prior to contacting with the at least one perturbagen. In some embodiments, the decrease in the number of progenitor cells is due to a change of cell state from a progenitor cell into a goblet cell and/or enterocyte.

In some embodiments, the methods described herein are such that the number of progenitor cells is increased. In embodiments, the increase in the number of progenitor cells is due in part to increased cell proliferation of the progenitor cells. In some embodiments, the increase in the number of progenitor cells is due in part to an increased lifespan of the progenitor cells. In embodiments, the increase in the number of progenitor cells is due in part to decreased cell death among the progenitor cells. In embodiments, the increase in the number of progenitor cells is relative to the number of progenitor cells in a population of progenitor cells that is not contacted with the at least one perturbagen. In some embodiments, the increase in the number of progenitor cells is relative to the number of progenitor cells in the population prior to contacting with the at least one perturbagen.

In some embodiments, the methods described herein are such that the number of goblet progenitors, goblet cells, Paneth cells, and/or enteroendocrine cells is increased after contacting the population of cells comprising a progenitor cell with the at least one perturbagen. In some embodiments, the methods described herein are such that the number of goblet progenitors and/or goblet cells is increased after contacting the population of cells comprising a progenitor cell with the at least one perturbagen. In embodiments, the number of Paneth cells and/or enteroendocrine cells is increased after contacting the population of cells comprising a progenitor cell with the at least one perturbagen. In embodiments, the number of goblet cells is increased after contacting the population of cells comprising a progenitor cell with the at least one perturbagen.

In some embodiments, the methods described herein are such that the ratio of the number of goblet progenitors, goblet cells, Paneth cells, and/or enteroendocrine cells to the number of progenitor cells is increased relative to the ratio obtained from a population of progenitor cells that is not contacted with the at least one perturbagen. In other embodiments, the ratio of the number goblet progenitors, goblet cells, Paneth cells, and/or enteroendocrine cells to the number of progenitor cells is increased relative to the ratio in the population of progenitor cells prior to contacting with the at least one perturbagen. In some embodiments, the ratio of the number of goblet progenitors to the number of progenitor cells is increased relative to the ratio obtained from a population of progenitor cells that is not contacted with the at least one perturbagen.

In embodiments, the ratio of the number of goblet progenitors to the number of progenitor cells is increased relative to the ratio in the population of progenitor cells prior to contacting with the at least one perturbagen. In embodiments, the ratio of the number of goblet progenitors to the number of enterocyte progenitors is increased relative to the ratio obtained from a population of progenitor cells that is not contacted with the at least one perturbagen.

In some embodiments, the ratio of the number of goblet progenitors to the number of enterocyte progenitors is increased relative to the ratio in the population of progenitor cells prior to contacting with the at least one perturbagen.

In embodiments, the ratio of the number of goblet progenitors to the number of enterocytes is increased relative to the ratio obtained from a population of progenitor cells that is not contacted with the at least one perturbagen.

According to some embodiments, the ratio of the number of goblet progenitors to the number of enterocytes is increased relative to the ratio in the population of progenitor cells prior to contacting with the at least one perturbagen. In some embodiments, the ratio of the number of goblet cells to the number of goblet progenitors is increased relative to the ratio obtained from a population of progenitor cells that is not contacted with the at least one perturbagen. In some embodiments, the ratio of the number of goblet cells to the number of goblet progenitors is increased relative to the ratio in the population of progenitor cells prior to contacting with the at least one perturbagen. In some embodiments, the ratio of the number of goblet cells to the number of intestinal stem cells is increased relative to the ratio obtained from a population of progenitor cells that is not contacted with the at least one perturbagen.

According to some embodiments, the ratio of the number of goblet cells to the number of intestinal stem cells is increased relative to the ratio in the population of progenitor cells prior to contacting with the at least one perturbagen.

In some embodiments, the ratio of the number of goblet cells to the number of Paneth cells is increased relative to the ratio obtained from a population of progenitor cells that is not contacted with the at least one perturbagen. In embodiments, the ratio of the number of goblet cells to the number of Paneth cells is increased relative to the ratio in the population of progenitor cells prior to contacting with the at least one perturbagen. In some embodiments, the ratio of the number of goblet cells to the number of enteroendocrine cells is increased relative to the ratio obtained from a population of progenitor cells that is not contacted with the at least one perturbagen. In embodiments, the ratio of the number of goblet cells to the number of enteroendocrine cells is increased relative to the ratio in the population of progenitor cells prior to contacting with the at least one perturbagen. In some embodiments, the ratio of the number of goblet cells to the number of enterocyte progenitors is increased relative to the ratio obtained from a population of progenitor cells that is not contacted with the at least one perturbagen.

According to some embodiments, the ratio of the number of goblet cells to the number of enterocyte progenitors is increased relative to the ratio in the population of progenitor cells prior to contacting with the at least one perturbagen.

In some embodiments, the ratio of the number of goblet cells, Paneth cells, and enteroendocrine cells to the number of enterocytes is increased relative to the ratio obtained from a population of progenitor cells that is not contacted with the at least one perturbagen. In some embodiments, the ratio of the number of goblet cells, Paneth cells, and enteroendocrine cells to the number of enterocytes is increased relative to the ratio in the population of progenitor cells prior to contacting with the at least one perturbagen.

According to some embodiments, the number of goblet progenitors, Paneth cells, enteroendocrine cells, enterocyte progenitors, and/or enterocytes is decreased. In some embodiments, the number of goblet progenitors is decreased.

In embodiments, the number of Paneth cells is decreased. In some embodiments, the number of enteroendocrine cells is decreased. In embodiments, the number of enterocyte progenitors is decreased. In some embodiments, the number of enterocytes is decreased.

According to some embodiments, the methods described herein are such that the number of goblet progenitors, goblet cells, Paneth cells, and/or enteroendocrine cells is increased. In some embodiments, the number of goblet progenitors is increased. In some embodiments, the number of goblet cells is increased. In other embodiments, the number of Paneth cells is increased. In some embodiments, the number of enteroendocrine cells is increased.

In some embodiments, the methods described here use at least one perturbagen selected from Table 2, or a variant thereof. In some embodiments, the at least one perturbagen comprises at least 2, at least 3, at least 4, at least 5, at least 6, at least 7, at least 8, at least 9, at least 10, or at least 11 perturbagens selected from Table 2, or variants thereof.

In some embodiments, the one or more genes designated as an “up” gene in the gene directionality column of Table 1 comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, or 58 or more genes. In embodiments, the one or more genes designated as an “up” gene in the gene directionality column of Table 1 comprises at least one of BIRC5, CCNB1, UBE2C, CDC20, CCNA2, S100A13, PMM2, GADD45B, GADD45A, RAP1GAP, TM9SF2, TMED10, STXBP1, GALE, KDELR2, ADGRE5, MTHFD2, FHL2, PYCR1, MVP, BAMBI, BAD, HMGCS1, ATP6V0B, FDFT1, HMGCR, EBP, ACLY, FGFR2, STAP2, XBP1, GFPT1, CDKN1A, HYOU1, ACBD3, COPB2, HERPUD1, NUCB2, CDC25B, BACE2, RGS2, RPN1, RAB27A, SLC35A1, RNH1, C2CD2L, ARHGAP1, SLC35B1, CRELD2, UFM1, ARFIP2, KIT, FAIM, NPDC1, TIMM17B, PDIA5, TM9SF3, and EDEM1. In some embodiments, the one or more genes designated as a “down” gene in the gene directionality column of Table 1 comprise 2 or more, 3 or more, 4 or more, 5 or more, 6 or more, 7 or more, 8 or more, 9 or more, 10 or more 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or ore, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or ore, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, or 81 or more genes. In embodiments, the one or more genes designated as a “down” gene in the gene directionality column of Table 1 comprise at least one of MIF, TRAP1, CETN3, CHEK2, RAE1, HES1, HADH, RFC2, MCM3, PCNA, HAT1, TOPBP1, EED, DDB2, PKIG, SNX7, DYRK3, CCNH, PHGDH, GLRX, CHP1, NR3C1, TSC22D3, EIF5, FOXO3, SOX4, H2AFV, GRB7, SNX6, NOTCH1, NET1, CD44, VDAC1, CREG1, PRSS23, FKBP4, MBNL1, MYC, MRPL12, CD320, G3BP1, CBR1, IFRD2, GRWD1, RPS5, TSPAN3, ADCK3, OXCT1, GPER1, PDGFA, TRIM2, APP, UGDH, PSME1, MTA1, RPA2, PGRMC1, PSIP1, HSPD1, PAICS, SDHB, NOLC1, ATP1B1, PLA2G4A, TOMM70A, ADH5, MRPL19, AARS, SOCS2, HEBP1, TIMM9, NIPSNAP1, OXA1L, MSRA, ARIDSB, BZW2, EDN1, POLR1C, CDK4, DCTD, and HSD17B10.

In embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In embodiments, contacting the population of cells comprising a progenitor cell occurs in vitro or ex vivo.

In embodiments, contacting the population of cells comprising a progenitor cell occurs in vivo in a subject. In embodiments, the subject is a human. In embodiments, the human is an adult human.

In some embodiments, the methods described herein are such that the change in cell state provides one or more of: a) increased secretion of mucus by a goblet cell and b) increased synthesis of one or more mucins, optionally selected from MUC2, MUC1, MUC3, or MUC17), trefoil factor peptides (TFF), resistin-like molecule β (RELMβ), and Fc-γ binding protein (FcγBP).

In yet another aspect, the present disclosure provides a perturbagen for use in any herein disclosed method.

In a further aspect, the present disclosure provides a pharmaceutical composition comprising perturbagen for use in any herein disclosed method.

In some aspects, the present disclosure is related to a method for promoting the formation of a goblet cell, or an immediate progenitor thereof, comprising: exposing a starting population of intestinal stem cells to a perturbation having a perturbation signature that promotes the transition of the starting population of intestinal stem cells into a M₀ cell or a goblet cell, wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1.

In some embodiments, the perturbation signature comprises an increase in expression and/or activity of one or more genes in the progenitor cell of an activation of a network module designated in the network module column of Table 1.

In embodiments, the activation of one or more genes of the network module designated in the network module column of Table 1 comprises modulating expression and/or activity of 2 or more genes within a network module.

In some embodiments, the perturbation signature comprises an increase in expression and/or activity in the progenitor cell of two or more genes designated as an “up” gene in the gene directionality column of Table 1. In embodiments, the perturbation signature comprises a decrease in expression and/or activity in the progenitor cell of two or more genes designated as a “down” gene in the gene directionality column of Table 1.

Embodiments associated with the above aspects are likewise relevant to the present aspect. In other words, each of the embodiments mentioned above for the above aspects may be revised/adapted to be applicable to the present aspect.

### Methods and Perturbagens for Treating a Disease or Disorder

The ability of a perturbagen to specifically promote one or more of goblet progenitors, goblet cells, Paneth cells, and/or enteroendocrine cells would be valuable in designing a therapeutic composition. As examples, for a disease characterized by a reduced number of one or more of goblet progenitors, goblet cells, Paneth cells, and/or enteroendocrine cells, a therapeutic composition comprising a perturbagen that increases the number of one or more of goblet progenitors, goblet cells, Paneth cells, and/or enteroendocrine cells could be beneficial. A disease that would benefit from increased numbers of goblet progenitors or goblet cells could be treated by a therapeutic composition comprising a perturbagen that increases the number of goblet progenitors or goblet cells.

Another aspect of the present disclosure is related to a method for treating a disease or disorder characterized by an abnormal number or abnormal function of goblet cells, comprising: (a) administering to a patient in need thereof a therapeutically effective amount of at least one perturbagen selected from Table 2, or a variant thereof, wherein the at least one perturbagen is capable of changing a gene signature in a progenitor cell or (b) administering to a patient in need thereof a cell, the cell having been contacted with at least one perturbagen selected from Table 2, or a variant thereof, wherein the at least one perturbagen is capable of changing a gene signature in a progenitor cell.

In some embodiments, the disease or disorder is caused by a goblet cell deficiency.

In some embodiments, the at least one perturbagen is administered on the basis of previously determining that the patient exhibits an abnormal number or abnormal function of goblet cells, or a disease or disorder characterized thereby. In embodiments, the administering, as described herein, is directed to the bone marrow of the patient. In embodiments, the administering is via intraosseous injection or intraosseous infusion. In embodiments, the administering the cell is via intravenous injection or intravenous infusion. In other embodiments, the administering of the cell is via intravenous injection or intravenous infusion. In some embodiments, the administering is simultaneously or sequentially to one or more mobilization agents. In some embodiments, the administering of the perturbagen is via oral, intravenous, intramuscular, intraperitoneal, subcutaneous, intra-articular injection, and/or infusion route. In embodiments, the delivery/administration of the perturbagen is via the gastrointestinal (GI) tract, optionally selected from the stomach, small intestine, duodenum, jejunum, ileum, large intestine, colon transversum, colon descendens, colon ascendens, colon sigmoidenum, cecum, and rectum.

In some embodiments, the perturbagen is formulated with a delayed-release coating, which is optionally enzyme-dependent.

In some embodiments, the disease or disorder that is treated by the methods described herein is selected from intestinal infection, colitis, inflammatory bowel disease (IBD), ulcerative colitis, cystic fibrosis, and cancer.

Yet another aspect of the present disclosure is related to a method for treating a disease or disorder characterized by an abnormal ratio of goblet cells to intestinal stem cells, comprising: (a) administering to a patient in need thereof at least one perturbagen selected from Table 2, or a variant thereof, wherein the at least one perturbagen is capable of changing a gene signature in an intestinal stem cell or (b) administering to a patient in need thereof a cell, the cell having been contacted with at least one perturbagen selected from Table 2, or a variant thereof, wherein the at least one perturbagen is capable of changing a gene signature in an intestinal stem cell. In some embodiments, the abnormal ratio comprises a decreased number of goblet cells and/or an increased number of intestinal stem cells. In embodiments, the abnormal ratio comprises an increased number of intestinal stem cells. In other embodiments, the abnormal ratio comprises a decreased number of goblet cells.

In some embodiments, the administering according to this aspect is directed to the bone marrow of the patient. In embodiments, the administering is via intraosseous injection or intraosseous infusion. In some embodiments, the administering the cell is via intravenous injection or intravenous infusion. In some embodiments, the administering is simultaneously or sequentially to one or more mobilization agents. In embodiments, the administering of the perturbagen is via oral, intravenous, intramuscular, intraperitoneal, subcutaneous, intra-articular injection, and/or infusion route. In some embodiments, the delivery/administration of the perturbagen is via the gastrointestinal (GI) tract, optionally selected from the stomach, small intestine, duodenum, jejunum, ileum, large intestine, colon transversum, colon descendens, colon ascendens, colon sigmoidenum, cecum, and rectum.

In some embodiments, the disease or disorder that is treated by according to this aspect of the disclosure is selected from intestinal infection, colitis, inflammatory bowel disease (IBD), ulcerative colitis, cystic fibrosis, and cancer.

In some embodiments, the at least one perturbagen is administered on the basis of previously determining that the patient exhibits the abnormal ratio of goblet cells to intestinal stem cells, or a disease or disorder characterized thereby.

In embodiments, the at least one perturbagen is capable of changing a gene signature in a progenitor cell.

In some embodiments, the present disclosure is related to selection of a patient. The patient is selected by steps comprising: obtaining from the patient having the disease or disorder a sample of cells comprising at least one intestinal stem cell; and contacting the sample of cells with least one perturbagen selected from Table 2, or a variant thereof, wherein the at least one perturbagen alters a gene signature in the sample of cells.

In some embodiments, the patient is selected by steps comprising: obtaining from a subject having the disease or disorder a sample of cells comprising at least one intestinal stem cell; and contacting the sample of cells with at least one perturbagen capable of altering a gene signature in an intestinal stem cell, wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1.

In embodiments, the patient is selected by steps comprising: obtaining from a subject having the disease or disorder a sample of cells comprising an intestinal stem cell; and contacting the sample of cells with at least one perturbagen selected from Table 2, or a variant thereof; wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1.

In some embodiments, the perturbagen causes an increase in expression and/or activity of one or more genes in the progenitor cell of an activation of a network module designated in the network module column of Table 1. In embodiments, the activation of one or more genes of the network module designated in the network module column of Table 1 comprises modulating expression and/or activity of 2 or more genes within a network module. In some embodiments, the perturbagen causes an increase in expression and/or activity in the progenitor cell of two or more genes designated as an “up” gene in the gene directionality column of Table 1. In embodiments, the perturbagen causes a decrease in expression and/or activity in the progenitor cell of two or more genes designated as a “down” gene in the gene directionality column of Table 1.

In some embodiments, the present disclosure is related to a method for selecting the patient comprising: obtaining from a subject having the disease or disorder a sample of cells comprising an intestinal stem cell; and contacting the sample of cells with least one perturbagen selected from Table 2, or a variant thereof, wherein when the at least one perturbagen alters a gene signature in the sample of cells, the subject is selected as a patient.

In some embodiments, the method for selecting the patient according to any of the methods described herein include a step of selecting a patient that includes obtaining from a subject, having the disease or disorder described herein, a sample of cells comprising an intestinal stem cell; and contacting the sample of cells with at least one perturbagen capable of altering a gene signature in an intestinal stem cell, wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

In some embodiments, the method for selecting the patient according to any of the methods described herein include obtaining from a subject, having the disease or disorder described herein, a sample of cells comprising an intestinal stem cell; and contacting the sample of cells with at least one perturbagen selected from Table 2, or a variant thereof; wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

In some embodiments, the perturbagen causes an increase in expression and/or activity of one or more genes in the progenitor cell of an activation of a network module designated in the network module column of Table 1. In embodiments, the activation of one or more genes of the network module designated in the network module column of Table 1 comprises modulating expression and/or activity of 2 or more genes within a network module. In some embodiments, the perturbagen causes an increase in expression and/or activity in the progenitor cell of two or more genes designated as an “up” gene in the gene directionality column of Table 1. In embodiments, the perturbagen causes a decrease in expression and/or activity in the progenitor cell of two or more genes designated as a “down” gene in the gene directionality column of Table 1.

In some embodiments, the present disclosure is related to the use of the perturbagen of Table 2, or a variant thereof in the manufacture of a medicament for treating a disease or disorder characterized by an abnormal ratio of goblet cells to intestinal stem cells. In embodiments, the present disclosure is related to the use of the perturbagen of Table 2, or a variant thereof in the manufacture of a medicament for treating a disease or disorder characterized by an abnormal ratio of goblet cells to enterocytes, Paneth cells and/or enteroendocrine cells.

In some aspects, the present disclosure is related to a method of identifying a candidate perturbation for promoting the transition of a starting population of intestinal stem cells into goblet cells or immediate progenitors thereof, the method comprising: exposing the starting population of intestinal stem cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of intestinal stem cells into goblet cells or immediate progenitors thereof following exposure of the population of cells to the perturbation; and identifying the perturbation as a candidate perturbation for promoting the transition of a population of intestinal stem cells into goblet cells or immediate progenitors thereof based on the perturbation signature, wherein the perturbation signature is an increase in expression and/or activity in the intestinal stem cell of one or more genes designated as an “up” gene in the gene directionality column of Table 1, and/or a decrease in expression and/or activity in the intestinal stem cell of one or more genes designated as a “down” gene in the gene directionality column of Table 1.

In some aspects, the present disclosure is related to a method for making a therapeutic agent for a disease or disorder selected from intestinal infection, colitis, inflammatory bowel disease (IBD), ulcerative colitis, cystic fibrosis, and cancer, comprising: (a) identifying a therapeutic agent for therapy according to a method of the disclosure and (b) formulating the therapeutic agent for the treatment of the disease or disorder.

Methods for determining the extension of the lifespan of a specific cell type or a reduction of cell death is well known in the art. As examples, markers for dying cells, e.g., caspases can be detected, or dyes for dead cells, e.g., methylene blue, may be used. Methods for counting cells are well known in the art. Non-limiting examples include hemocytometry, flow cytometry, and cell sorting techniques, e.g., fluorescence activated cell sorting (FACS).

### Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of stem cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter a gene signature in a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int. J. Hematol. Oncol. Stem Cell Res. 2017 Jan. 1; 11(1): 78-88; Hopman and DiPersio “Advances in Stem Cell Mobilization.” Blood Review, 2014, 28(1): 31-40; and Kim “Hematopoietic stem cell mobilization: current status and future perspective.” Blood Res. 2017 June; 52(2): 79-81. The content of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any perturbagen disclosed herein can be administered in a single daily dose, or the total daily dosage can be administered in divided doses of two, three or four times daily. Furthermore, any perturbagen disclosed herein can be administered continuously rather than intermittently throughout the dosage regimen.

### Pharmaceutical Compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

Combination therapies, comprising more than one perturbagen, can be co-delivered in a single delivery vehicle or delivery device. Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any perturbagens disclosed herein is formulated in accordance with routine procedures as a pharmaceutical composition adapted for a mode of administration disclosed herein.

Yet another aspect of the present disclosure is a perturbagen capable of causing a change in a gene signature.

In an aspect, the present disclosure provides a perturbagen capable of causing a change in cell fate.

In another aspect, the present disclosure provides a perturbagen capable of causing a change in a gene signature and a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

The instant disclosure also provides certain embodiments as follows:

Embodiment 1: A method for directing a change in cell state of a progenitor cell comprising: contacting a population of cells comprising a progenitor cell with at least one perturbagen selected from Table 2, or a variant thereof; wherein the at least one perturbagen is capable of altering a gene signature in the progenitor cell; and wherein the progenitor cell is an intestinal stem cell.

Embodiment 2: A method for directing a change in cell state of a progenitor cell, comprising: contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

Embodiment 3: A method for directing a change in cell state of a progenitor cell, comprising: contacting a population of cells comprising a progenitor cell with at least one perturbagen selected from Table 2, or a variant thereof, and capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

Embodiment 4: The method of any one of Embodiments 1-3, wherein altering the gene signature comprises a change in expression and/or activity of one or more genes in the progenitor cell of a network module designated in the network module column of Table 1.

Embodiment 5: The method of any one of Embodiments 1-4, wherein the activation of one or more genes of the network module designated in the network module column of Table 1 comprises modulating expression and/or activity of 2 or more genes within a network module.

Embodiment 6: The method of any one of Embodiments 1-5, wherein altering the gene signature comprises an increase in expression and/or activity in the progenitor cell of two or more genes designated as an “up” gene in the gene directionality column of Table 1.

Embodiment 7: The method of any one of Embodiments 1-6, wherein altering the gene signature comprises a decrease in expression and/or activity in the progenitor cell of two or more genes designated as a “down” gene in the gene directionality column of Table 1.

Embodiment 8: The method of any one of Embodiments 1 to 3, wherein the change in cell state provides an increase in the number of one or more of goblet progenitors, goblet cells, Paneth cells, and enteroendocrine cells.

Embodiment 9: The method of Embodiment 8, wherein the change in cell state provides an increase in the number of goblet cells.

Embodiment 10: The method of Embodiment 9, wherein the increase in the number of goblet cells is relative to the number of goblets cells obtained from a population of progenitor cells that is not contacted with the at least one perturbagen.

Embodiment 11: The method of Embodiment 9, wherein the increase in the number of goblet cells is relative to the number of goblets cells obtained from a population of progenitor cells prior to contacting with the at least one perturbagen.

Embodiment 12: The method of any one of Embodiments 9 to 11, wherein the ratio of the number of goblet cells to the number of enterocytes is increased relative to the ratio obtained from a population of progenitor cells that is not contacted with the at least one perturbagen.

Embodiment 13: The method of any one of Embodiments 9 to 11, wherein the ratio of the number of goblet cells to the number of enterocytes is increased relative to the ratio in the population of progenitor cells prior to contacting with the at least one perturbagen.

Embodiment 14: The method of Embodiment 9, wherein the ratio of the number of goblet cells to the number of progenitor cells is increased relative to the ratio obtained from a population of progenitor cells that is not contacted with the at least one perturbagen.

Embodiment 15: The method of Embodiment 9, wherein the ratio of the number of goblet cells to the number of progenitor cells is increased relative to the ratio in the population of progenitor cells prior to contacting with the at least one perturbagen.

Embodiment 16: The method of any one of Embodiments 8 to 12, wherein the increase in the number of goblet cells is due in part to increased cell proliferation of the goblet cells.

Embodiment 17: The method of any one of Embodiments 8 to 12, wherein the increase in the number of goblet cells is due in part to an increased lifespan of the goblet cells.

Embodiment 18: The method of any one of Embodiments 8 to 12, wherein the increase in the number of goblet cells is due in part to reduced cell death among the goblet cells.

Embodiment 19: The method of any one of Embodiments 1 to 18, wherein the number of progenitor cells is decreased.

Embodiment 20: The method of Embodiment 19, wherein the decrease in the number of progenitor cells is due in part to decreased cell proliferation of the progenitor cells.

Embodiment 21: The method of Embodiment 19 or Embodiment 20, wherein the decrease in the number of progenitor cells is due in part to a decreased lifespan of the progenitor cells.

Embodiment 22: The method of any one of Embodiments 19 to 21, wherein the decrease in the number of progenitor cells is due in part to increased cell death among the progenitor cells.

Embodiment 23: The method of any one of Embodiments 19 to 22, wherein the decrease in the number of progenitor cells is relative to the number of progenitor cells in a population of progenitor cells that is not contacted with the at least one perturbagen.

Embodiment 24: The method of any one of Embodiments 19 to 22, wherein the decrease in the number of progenitor cells is relative to the number of progenitor cells in the population prior to contacting with the at least one perturbagen.

Embodiment 25: The method of any one of Embodiments 19 to 22, wherein the decrease in the number of progenitor cells is due to a change of cell state from a progenitor cell into a goblet cell.

Embodiment 26: The method of any one of Embodiments 1 to 25, wherein the number of progenitor cells is increased.

Embodiment 27: The method of Embodiment 26, wherein the increase in the number of progenitor cells is due in part to increased cell proliferation of the progenitor cells.

Embodiment 28: The method of Embodiment 26 or Embodiment 27, wherein the increase in the number of progenitor cells is due in part to an increased lifespan of the progenitor cells.

Embodiment 29: The method of any one of Embodiments 26 to 28, wherein the increase in the number of progenitor cells is due in part to decreased cell death among the progenitor cells.

Embodiment 30: The method of any one of Embodiments 26 to 29, wherein the increase in the number of progenitor cells is relative to the number of progenitor cells in a population of progenitor cells that is not contacted with the at least one perturbagen.

Embodiment 31: The method of any one of Embodiments 26 to 29, wherein the increase in the number of progenitor cells is relative to the number of progenitor cells in the population prior to contacting with the at least one perturbagen.

Embodiment 32: The method of any one of Embodiments 1 to 18, wherein the number of goblet progenitors, goblet cells, Paneth cells, and/or enteroendocrine cells is increased after contacting the population of cells comprising a progenitor cell with the at least one perturbagen.

Embodiment 33: The method of any one of Embodiments 1 to 18, wherein the number of goblet progenitors and/or goblet cells is increased after contacting the population of cells comprising a progenitor cell with the at least one perturbagen.

Embodiment 34: The method of any one of Embodiments 1 to 18, wherein the number of Paneth cells and/or enteroendocrine cells is increased after contacting the population of cells comprising a progenitor cell with the at least one perturbagen.

Embodiment 35: The method of any one of Embodiments 1 to 18, wherein the number of goblet cells is increased after contacting the population of cells comprising a progenitor cell with the at least one perturbagen.

Embodiment 36: The method of Embodiment 32, wherein the ratio of the number of goblet progenitors, goblet cells, Paneth cells, and/or enteroendocrine cells to the number of progenitor cells is increased relative to the ratio obtained from a population of progenitor cells that is not contacted with the at least one perturbagen.

Embodiment 37: The method of Embodiment 32, wherein the ratio of the number goblet progenitors, goblet cells, Paneth cells, and/or enteroendocrine cells to the number of progenitor cells is increased relative to the ratio in the population of progenitor cells prior to contacting with the at least one perturbagen.

Embodiment 38: The method of any one of Embodiments 1 to 18, wherein the ratio of the number of goblet progenitors to the number of progenitor cells is increased relative to the ratio obtained from a population of progenitor cells that is not contacted with the at least one perturbagen.

Embodiment 39: The method of Embodiment 38, wherein the ratio of the number of goblet progenitors to the number of progenitor cells is increased relative to the ratio in the population of progenitor cells prior to contacting with the at least one perturbagen.

Embodiment 40: The method of any one of Embodiments 1 to 18, wherein the ratio of the number of goblet progenitors to the number of enterocyte progenitors is increased relative to the ratio obtained from a population of progenitor cells that is not contacted with the at least one perturbagen.

Embodiment 41: The method of Embodiment 40, wherein the ratio of the number of goblet progenitors to the number of enterocyte progenitors is increased relative to the ratio in the population of progenitor cells prior to contacting with the at least one perturbagen.

Embodiment 42: The method of any one of Embodiments 1 to 18, wherein the ratio of the number of goblet progenitors to the number of enterocytes is increased relative to the ratio obtained from a population of progenitor cells that is not contacted with the at least one perturbagen.

Embodiment 43: The method of Embodiment 42, wherein the ratio of the number of goblet progenitors to the number of enterocytes is increased relative to the ratio in the population of progenitor cells prior to contacting with the at least one perturbagen.

Embodiment 44: The method of any one of Embodiments 1 to 18, wherein the ratio of the number of goblet cells to the number of goblet progenitors is increased relative to the ratio obtained from a population of progenitor cells that is not contacted with the at least one perturbagen.

Embodiment 45: The method of Embodiment 44, wherein the ratio of the number of goblet cells to the number of goblet progenitors is increased relative to the ratio in the population of progenitor cells prior to contacting with the at least one perturbagen.

Embodiment 46: The method of any one of Embodiments 1 to 18, wherein the ratio of the number of goblet cells to the number of intestinal stem cells is increased relative to the ratio obtained from a population of progenitor cells that is not contacted with the at least one perturbagen.

Embodiment 47: The method of Embodiment 46, wherein the ratio of the number of goblet cells to the number of intestinal stem cells is increased relative to the ratio in the population of progenitor cells prior to contacting with the at least one perturbagen.

Embodiment 48: The method of any one of Embodiments 1 to 18, wherein the ratio of the number of goblet cells to the number of Paneth cells is increased relative to the ratio obtained from a population of progenitor cells that is not contacted with the at least one perturbagen.

Embodiment 49: The method of Embodiment 48, wherein the ratio of the number of goblet cells to the number of Paneth cells is increased relative to the ratio in the population of progenitor cells prior to contacting with the at least one perturbagen.

Embodiment 50: The method of any one of Embodiments 1 to 18, wherein the ratio of the number of goblet cells to the number of enteroendocrine cells is increased relative to the ratio obtained from a population of progenitor cells that is not contacted with the at least one perturbagen.

Embodiment 51: The method of Embodiment 50, wherein the ratio of the number of goblet cells to the number of enteroendocrine cells is increased relative to the ratio in the population of progenitor cells prior to contacting with the at least one perturbagen.

Embodiment 52: The method of any one of Embodiments 1 to 18, wherein the ratio of the number of goblet cells to the number of enterocyte progenitors is increased relative to the ratio obtained from a population of progenitor cells that is not contacted with the at least one perturbagen.

Embodiment 53: The method of Embodiment 52, wherein the ratio of the number of goblet cells to the number of enterocyte progenitors is increased relative to the ratio in the population of progenitor cells prior to contacting with the at least one perturbagen.

Embodiment 54: The method of any one of Embodiments 1 to 18, wherein the ratio of the number of goblet cells, Paneth cells, and enteroendocrine cells to the number of enterocytes is increased relative to the ratio obtained from a population of progenitor cells that is not contacted with the at least one perturbagen.

Embodiment 55: The method of Embodiment 54, wherein the ratio of the number of goblet cells, Paneth cells, and enteroendocrine cells to the number of enterocytes is increased relative to the ratio in the population of progenitor cells prior to contacting with the at least one perturbagen.

Embodiment 56: The method of any one of Embodiments 1 to 31, wherein the number of goblet progenitors, Paneth cells, enteroendocrine cells, enterocyte progenitors, and/or enterocytes is decreased.

Embodiment 57: The method of any of one Embodiments 1 to 31, wherein the number of goblet progenitors is decreased.

Embodiment 58: The method of any one of Embodiments 1 to 31, wherein the number of Paneth cells is decreased.

Embodiment 59: The method of any one of Embodiments 1 to 31, wherein the number of enteroendocrine cells is decreased.

Embodiment 60: The method of any one of Embodiments 1 to 31, wherein the number of enterocyte progenitors is decreased.

Embodiment 61: The method of any one of Embodiments 1 to 31, wherein the number of enterocytes is decreased.

Embodiment 62: The method of any one of Embodiments 1 to 31, wherein the number of goblet progenitors, goblet cells, Paneth cells, and/or enteroendocrine cells is increased.

Embodiment 63: The method of any one of Embodiments 1 to 31, wherein the number of goblet progenitors is increased.

Embodiment 64: The method of any one of Embodiments 1 to 31, wherein the number of goblet cells is increased.

Embodiment 65: The method of any one of Embodiments 1 to 31, wherein the number of Paneth cells is increased.

Embodiment 66: The method of any one of Embodiments 1 to 31, wherein the number of enteroendocrine cells is increased.

Embodiment 67: The method of any one of Embodiments 1-66, wherein the at least one perturbagen selected from Table 2, or a variant thereof, comprises at least 2, at least 3, at least 4, at least 5, at least 6, at least 7, at least 8, at least 9, at least 10, or at least 11 perturbagens selected from Table 2, or variants thereof.

Embodiment 68: The method of any one of Embodiments 1-66, wherein the one or more genes are selected from the genes designated as an “up” gene in the gene directionality column of Table 1 comprises 2 or more, 3 or more, 4 or more, 5 or more, 6 or more, 7 or more, 8 or more, 9 or more, 10 genes or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, or 58 or more genes selected from the genes designated as an “up” gene in the gene directionality column of Table 1.

Embodiment 69: The method of Embodiment 68, wherein the one or more genes selected from Table 1 comprises at least one of BIRC5, CCNB1, UBE2C, CDC20, CCNA2, S100A13, PMM2, GADD45B, GADD45A, RAP1GAP, TM9SF2, TMED10, STXBP1, GALE, KDELR2, ADGRE5, MTHFD2, FHL2, PYCR1, MVP, BAMBI, BAD, HMGCS1, ATP6V0B, FDFT1, HMGCR, EBP, ACLY, FGFR2, STAP2, XBP1, GFPT1, CDKN1A, HYOU1, ACBD3, COPB2, HERPUD1, NUCB2, CDC25B, BACE2, RGS2, RPN1, RAB27A, SLC35A1, RNH1, C2CD2L, ARHGAP1, SLC35B1, CRELD2, UFM1, ARFIP2, KIT, FAIM, NPDC1, TIMM17B, PDIA5, TM9SF3, and EDEM1.

Embodiment 70: The method of any one of Embodiments 1-66, wherein the one or more genes are selected from the genes designated as a “down” gene in the gene directionality column of Table 1 comprises 2 or more, 3 or more, 4 or more, 5 or more, 6 or more, 7 or more, 8 or more, 9 or more, 10 genes or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or ore, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or ore, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, or 81 or more genes selected from the genes designated as a “down” gene in the gene directionality column of Table 1.

Embodiment 71: The method of Embodiment 70, wherein the one or more genes selected from Table 1 comprises at least one of MIF, TRAP1, CETN3, CHEK2, RAE1, HES1, HADH, RFC2, MCM3, PCNA, HAT1, TOPBP1, EED, DDB2, PKIG, SNX7, DYRK3, CCNH, PHGDH, GLRX, CHP1, NR3C1, TSC22D3, EIF5, FOXO3, SOX4, H2AFV, GRB7, SNX6, NOTCH1, NET1, CD44, VDAC1, CREG1, PRSS23, FKBP4, MBNL1, MYC, MRPL12, CD320, G3BP1, CBR1, IFRD2, GRWD1, RPS5, TSPAN3, ADCK3, OXCT1, GPER1, PDGFA, TRIM2, APP, UGDH, PSME1, MTA1, RPA2, PGRMC1, PSIP1, HSPD1, PAICS, SDHB, NOLC1, ATP1B1, PLA2G4A, TOMM70A, ADH5, MRPL19, AARS, SOCS2, HEBP1, TIMM9, NIPSNAP1, OXA1L, MSRA, ARIDSB, BZW2, EDN1, POLR1C, CDK4, DCTD, and HSD17B10.

Embodiment 72: The method of any one of Embodiments 1 to 71, wherein contacting the population of progenitor cells occurs in vitro or ex vivo.

Embodiment 73: The method of any one of Embodiments 1 to 71, wherein contacting the population of progenitor cells occurs in vivo in a subject.

Embodiment 74: The method of Embodiment 73, wherein the subject is a human.

Embodiment 75: The method of any one of Embodiments 1 to 71, wherein the change in cells state provides one or more of: (a) increased secretion of mucus by a goblet cell and (b) increased synthesis of one or more mucins, optionally selected from MUC2, MUC1, MUC3, or MUC17), trefoil factor peptides (TFF), resistin-like molecule β (RELMβ), and Fc-γ binding protein (FcγBP).

Embodiment 76: A perturbagen for use in the method of any one of Embodiments 1 to 75.

Embodiment 77: A pharmaceutical composition comprising the perturbagen of Embodiment 77.

Embodiment 78: A method for promoting the formation of a goblet cell, or an immediate progenitor thereof, comprising: exposing a starting population of intestinal stem cells to a perturbation having a perturbation signature that promotes the transition of the starting population of intestinal stem cells into a M₀ cell or a goblet cell, wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1

Embodiment 79: The method of Embodiment 78, wherein the perturbation signature comprises an increase in expression and/or activity of one or more genes in the progenitor cell of an activation of a network module designated in the network module column of Table 1.

Embodiment 80: The method of Embodiment 78 or Embodiment 79, wherein the activation of one or more genes of the network module designated in the network module column of Table 1 comprises modulating expression and/or activity of 2 or more genes within a network module.

Embodiment 81: The method of any one of Embodiments 78-80, wherein the perturbation signature comprises an increase in expression and/or activity in the progenitor cell of two or more genes designated as an “up” gene in the gene directionality column of Table 1.

Embodiment 82: The method of any one of Embodiments 78-81, wherein the perturbation signature comprises a decrease in expression and/or activity in the progenitor cell of two or more genes designated as a “down” gene in the gene directionality column of Table 1.

Embodiment 83: A method for treating a disease or disorder characterized by an abnormal number or function of goblet cells, comprising: (a) administering to a patient in need thereof a therapeutically effective amount of at least one perturbagen selected from Table 2, or a variant thereof, wherein the at least one perturbagen is capable of changing a gene signature in a progenitor cell or (b) administering to a patient in need thereof a cell, the cell having been contacted with at least one perturbagen selected from Table 2, or a variant thereof, wherein the at least one perturbagen is capable of changing a gene signature in a progenitor cell.

Embodiment 84: The method of Embodiment 83, wherein the disease or disorder is caused by a goblet cell deficiency.

Embodiment 85: The method of Embodiments 83 or 84, wherein the administering is directed to the bone marrow of the patient.

Embodiment 86: The method of Embodiment 85, wherein the administering is via intraosseous injection or intraosseous infusion.

Embodiment 87: The method of any one of Embodiments 83 to 86, wherein the administering the cell is via intravenous injection or intravenous infusion.

Embodiment 88: The method of any one of Embodiments 83 to 87, wherein the administering is simultaneously or sequentially to one or more mobilization agents.

Embodiment 89: The method of Embodiment 83, wherein the administering of the perturbagen is via oral, intravenous, intramuscular, intraperitoneal, subcutaneous, intra-articular injection, and/or infusion route.

Embodiment 90: The method of Embodiment 89, wherein the delivery is the gastrointestinal (GI) tract, optionally selected from the stomach, small intestine, duodenum, jejunum, ileum, large intestine, colon transversum, colon descendens, colon ascendens, colon sigmoidenum, cecum, and rectum.

Embodiment 91: The method of Embodiments 89 or 90, wherein the perturbagen is formulated with a delayed-release coating, which is optionally enzyme-dependent.

Embodiment 92: The method of Embodiment 83, wherein the disease or disorder is selected from intestinal infection, colitis, inflammatory bowel disease (IBD), ulcerative colitis, cystic fibrosis, and cancer.

Embodiment 93: The method of Embodiment 83, wherein at least one perturbagen is administered on the basis of previously determining that the patient exhibits an abnormal number or function of goblet cells, or a disease or disorder characterized thereby.

Embodiment 94: A method for treating a disease or disorder characterized by an abnormal ratio of goblet cells to intestinal stem cells, comprising: (a) administering to a patient in need thereof at least one perturbagen selected from Table 2, or a variant thereof, wherein the at least one perturbagen is capable of changing a gene signature in an intestinal stem cell or (b) administering to a patient in need thereof a cell, the cell having been contacted with at least one perturbagen selected from Table 2, or a variant thereof, wherein the at least one perturbagen is capable of changing a gene signature in an intestinal stem cell.

Embodiment 95: The method of Embodiment 94, wherein the abnormal ratio comprises a decreased number of goblet cells and/or an increased number of intestinal stem cells.

Embodiment 96: The method of Embodiment 95, wherein the abnormal ratio comprises an increased number of intestinal stem cells.

Embodiment 97: The method of Embodiment 95, wherein the abnormal ratio comprises a decreased number of goblet cells.

Embodiment 98: The method of Embodiment 94, wherein the administering is directed to the bone marrow of the patient.

Embodiment 99: The method of Embodiment 94, wherein the administering is via intraosseous injection or intraosseous infusion.

Embodiment 100: The method of any one of Embodiments 94 to 99, wherein the administering the cell is via intravenous injection or intravenous infusion.

Embodiment 101: The method of any one of Embodiments 94 to 100, wherein the administering is simultaneously or sequentially to one or more mobilization agents.

Embodiment 102: The method of Embodiment 94, wherein the administering of the perturbagen is via oral, intravenous, intramuscular, intraperitoneal, subcutaneous, intra-articular injection, and/or infusion route.

Embodiment 103: The method of Embodiment 102, wherein the delivery is the gastrointestinal (GI) tract, optionally selected from the stomach, small intestine, duodenum, jejunum, ileum, large intestine, colon transversum, colon descendens, colon ascendens, colon sigmoidenum, cecum, and rectum.

Embodiment 104: The method of Embodiment 94, wherein the disease or disorder is selected from intestinal infection, colitis, inflammatory bowel disease (IBD), ulcerative colitis, cystic fibrosis, and cancer.

Embodiment 105: The method of Embodiment 94, wherein at least one perturbagen is administered on the basis of previously determining that the patient exhibits the abnormal ratio of goblet cells to intestinal stem cells, or a disease or disorder characterized thereby.

Embodiment 106: The method of any one of Embodiments 94 to 105, wherein the at least one perturbagen is capable of changing a gene signature in a progenitor cell.

Embodiment 107: The method of any one of Embodiments 94 to 106, wherein the patient is selected by steps comprising: obtaining from the patient having the disease or disorder a sample of cells comprising at least one intestinal stem cell; and contacting the sample of cells with least one perturbagen selected from Table 2, or a variant thereof, wherein the at least one perturbagen alters a gene signature in the sample of cells.

Embodiment 108: The method of any one of Embodiments 94 to 106, wherein the patient is selected by steps comprising: obtaining from a subject having the disease or disorder a sample of cells comprising at least one intestinal stem cell; and contacting the sample of cells with at least one perturbagen capable of altering a gene signature in an intestinal stem cell, wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1.

Embodiment 109: The method of Embodiment 108, wherein the perturbagen causes an increase in expression and/or activity of one or more genes in the progenitor cell of an activation of a network module designated in the network module column of Table 1.

Embodiment 110: The method of Embodiment 108 or Embodiment 109, wherein the activation of one or more genes of the network module designated in the network module column of Table 1 comprises modulating expression and/or activity of 2 or more genes within a network module.

Embodiment 111: The method of any one of Embodiments 108-110, wherein the perturbagen causes an increase in expression and/or activity in the progenitor cell of two or more genes designated as an “up” gene in the gene directionality column of Table 1.

Embodiment 112: The method of any one of Embodiments 108-111, wherein the perturbagen causes a decrease in expression and/or activity in the progenitor cell of two or more genes designated as a “down” gene in the gene directionality column of Table 1.

Embodiment 113: The method of any one of Embodiments 94 to 106, wherein the patient is selected by steps comprising: obtaining from a subject having the disease or disorder a sample of cells comprising an intestinal stem cell; and contacting the sample of cells with at least one perturbagen selected from Table 2, or a variant thereof; wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1.

Embodiment 114: The method of Embodiment 113, wherein the perturbagen causes an increase in expression and/or activity of one or more genes in the progenitor cell of an activation of a network module designated in the network module column of Table 1.

Embodiment 115: The method of Embodiment 113 or Embodiment 114, wherein the activation of one or more genes of the network module designated in the network module column of Table 1 comprises modulating expression and/or activity of 2 or more genes within a network module.

Embodiment 116: The method of any one of Embodiments 113-115, wherein the perturbagen causes an increase in expression and/or activity in the progenitor cell of two or more genes designated as an “up” gene in the gene directionality column of Table 1.

Embodiment 117: The method of any one of Embodiments 113-116, wherein the perturbagen causes a decrease in expression and/or activity in the progenitor cell of two or more genes designated as a “down” gene in the gene directionality column of Table 1.

Embodiment 118: A method for selecting the patient of any one of Embodiments 94 to 106, comprising: obtaining from a subject having the disease or disorder a sample of cells comprising an intestinal stem cell; and contacting the sample of cells with least one perturbagen selected from Table 2, or a variant thereof, wherein when the at least one perturbagen alters a gene signature in the sample of cells, the subject is selected as a patient.

Embodiment 119: A method for selecting the patient of any one of Embodiments 94 to 106, comprising: obtaining from a subject having the disease or disorder a sample of cells comprising an intestinal stem cell; and contacting the sample of cells with at least one perturbagen capable of altering a gene signature in an intestinal stem cell, wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

Embodiment 120: The method of Embodiment 119, wherein altering the gene signature comprises a change in expression and/or activity of one or more genes in the progenitor cell of a network module designated in the network module column of Table 1.

Embodiment 121: The method of any one of Embodiment 119 or Embodiment 120, wherein the activation of one or more genes of the network module designated in the network module column of Table 1 comprises modulating expression and/or activity of 2 or more genes within a network module.

Embodiment 122: The method of any one of Embodiments 119-121, wherein altering the gene signature comprises an increase in expression and/or activity in the progenitor cell of two or more genes designated as an “up” gene in the gene directionality column of Table 1.

Embodiment 123: The method of any one of Embodiments 119-122, wherein altering the gene signature comprises a decrease in expression and/or activity in the progenitor cell of two or more genes designated as a “down” gene in the gene directionality column of Table 1.

Embodiment 124: A method for selecting the patient of any one of Embodiments 94 to 106, comprising: obtaining from a subject having the disease or disorder a sample of cells comprising an intestinal stem cell; and contacting the sample of cells with at least one perturbagen selected from Table 2, or a variant thereof; wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

Embodiment 125: The method of Embodiment 124, wherein the perturbagen causes an increase in expression and/or activity of one or more genes in the progenitor cell of an activation of a network module designated in the network module column of Table 1.

Embodiment 126: The method of Embodiment 124 or Embodiment 125, wherein the activation of one or more genes of the network module designated in the network module column of Table 1 comprises modulating expression and/or activity of 2 or more genes within a network module.

Embodiment 127: The method of any one of Embodiments 124-126, wherein the perturbagen causes an increase in expression and/or activity in the progenitor cell of two or more genes designated as an “up” gene in the gene directionality column of Table 1.

Embodiment 128: The method of any one of Embodiments 124-127, wherein the perturbagen causes a decrease in expression and/or activity in the progenitor cell of two or more genes designated as a “down” gene in the gene directionality column of Table 1.

Embodiment 129: Use of the perturbagen of Table 2, or a variant thereof in the manufacture of a medicament for treating a disease or disorder characterized by an abnormal ratio of goblet cells to intestinal stem cells.

Embodiment 130: Use of the perturbagen of Table 2, or a variant thereof in the manufacture of a medicament for treating a disease or disorder characterized by an abnormal ratio of goblet cells to enterocytes, Paneth cells and/or enteroendocrine cells.

Embodiment 131: A method of identifying a candidate perturbation for promoting the transition of a starting population of intestinal stem cells into goblet cells or immediate progenitors thereof, the method comprising: exposing the starting population of intestinal stem cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of intestinal stem cells into goblet cells or immediate progenitors thereof following exposure of the population of cells to the perturbation; and identifying the perturbation as a candidate perturbation for promoting the transition of a population of intestinal stem cells into goblet cells or immediate progenitors thereof based on the perturbation signature, wherein the perturbation signature is an increase in expression and/or activity in the intestinal stem cell of one or more genes designated as an “up” gene in the gene directionality column of Table 1, and/or a decrease in expression and/or activity in the intestinal stem cell of one or more genes designated as a “down” gene in the gene directionality column of Table 1.

Embodiment 132: The method of Embodiment 131, wherein the perturbation signature is an increase in expression and/or activity in the progenitor cell of an activation of a network module designated in the network module column of Table 1.

Embodiment 133: The method of Embodiment 131 or Embodiment 132, wherein the activation of one or more genes of the network module designated in the network module column of Table 1 comprises modulating expression and/or activity of 2 or more genes within a network module.

Embodiment 134: The method of any one of Embodiments 131-133, wherein the perturbation signature is an increase in expression and/or activity in the progenitor cell of two or more genes designated as an “up” gene in the gene directionality column of Table 1.

Embodiment 135: The method of any one of Embodiments 131-134, wherein the perturbation signature is a decrease in expression and/or activity in the progenitor cell of two or more genes designated as a “down” gene in the gene directionality column of Table 1.

Embodiment 136: A method for making a therapeutic agent for a disease or disorder selected from intestinal infection, colitis, inflammatory bowel disease (IBD), ulcerative colitis, cystic fibrosis, and cancer, comprising: (a) identifying a candidate perturbation according to the method of Embodiment 131 and (b) formulating the candidate perturbation as a therapeutic agent for the treatment of the disease or disorder.

### Muco-Obstructive Diseases

The human respiratory system includes two vital regions: the conductive airways and the respiratory region. The airway is further divided into nasal cavity, sinuses, nasopharynx, oropharynx, larynx, trachea, bronchi, and bronchioles. The respiratory region consists of respiratory bronchioles, alveolar ducts, and alveolar sacs. The airway of the human lung consists of highly branched, tree-like system of tubes connected to a single trachea, pluralities of bronchioles and terminating in millions of alveoli. The intralobar airways supported by cartilage are bronchi, the smaller branches lacking cartilage are bronchioles. The human trachea, bronchi and bronchioles>1 mm in diameter are lined up by a pseudostratified epithelium made up of basal cells, secretory cells such as club cells and goblet cells, ciliated cells, inocytes, Tuft cells, and neuroendocrine cells acting as sensory cells. Mucous goblet cells predominate in the larger airways, and club cells reside in the smaller airways. In addition to epithelial cells, the airway system contains various mesenchylmal cell types including smooth muscle cells, peribronchial fibroblasts, myofibroblasts, AEC2 associated fibroblasts that support epithelia homeostasis and lung functions.

Individual neuroendocrine cell and neuroendocrine bodies (NEBs) are scattered in the larger airways and increase distally. Cartilage, smooth muscle and stromal cells are associated with intralobar airways down to the small bronchioles. The airway of the respiratory smallest bronchioles of human lung are covered by a cuboidal epithelium.

This epithelium lacks basal cells and contains ciliated cells, secretory cells and neuroendocrine cells that are usually clustered in neuroendocrine bodies (NEBs). The alveoli of human are composed of two functional distinct cell types, flat and extended alveolar type I cells (AEC1) to allow gas exchange and cuboidal alveolar type II cells (AEC2) that are stem cells for surface protein production and secretion.

Although airway tissue shows limited turnover in homeostasis, damage to the respiratory epithelium can be repaired by resident tissue stem cells. For example, under injury, myoepithelial cells resided in submucosal glands (SMGs) can differentiate into basal cells and serous cells. Myoepithelial cells also can differentiate into mucous cells.

Defects in the homeostatic and reparative mechanism may lead to diseases such as chronic obstructive pulmonary disease (COPD), asthma, fibrosis and cancer. Increases in goblet cells are observed upon immune stimuli and in diseases like COPD. Lineage tracing studies show that goblet cells can arise from Scgb1a1+ secretory cells and trans-differentiation of Foxj1+ciliated cells to goblet cells was observed upon smoke exposure in culture.

Goblet cell hyperplasia (GCH), squamous metaplasia, and chronic mucus hypersecretion, hallmarks of mucoobstructive disease and asthma, significantly contribute to disease morbidity. Goblet hyperplasia is characterized by increased numbers of goblet cells, decreased numbers of ciliated cells. Squamous metaplasia is characterized by multiple layers of basal cells that give rise to keratinized squamous cells. However, current treatments do not address the epithelial cell imbalance, and primarily entail immunosuppression or mucus hydration. Restoring differentiation to mucus-clearing ciliated cells while simultaneously suppressing differentiation to mucus-secreting goblet cells would help reduce total mucus production, which would furthermore ameliorate chronic infections and inflammation. Goblet and ciliated cells arise from a common progenitor, basal cells, therefore the present disclosure provides a composition comprising at least one perturbagen that would decrease differentiation to goblet cells, increase differentiation to ciliated cells without impacting other epithelial lineages.

The present disclosure relates to treat diseases or disorders characterized by abnormal numbers, ratios or bodily distributions of epithelial cells in the airway of human lung including goblet and ciliated cells, or immediate progenitors thereof with respect to each other.

### Genes Signatures

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may be useful to first characterize the cell type or cells of a specific lineage by proteins that are characteristic of the cell type or cells of a specific lineage. Illustrative protein markers for airway epithelial cell lineage include Trp63, Ngfr, pdpn, Krt5, and other protein markers such as Krt8, Krt14, Scgb1a1, Atpv1b1, Scgb3a2, Upka3, Cyp2f2. (Hogan et al., Cell Stem Cell, 2014, vol. 15, pp. 123-138). Illustrative markers for basal cells markers include NPPC, CTGF, DST, KRT15, CYR61, S100A2, KRT5, KRT17, BCAM, IGFBP3, CLCA2, SERPINB4, MT1X, SERPINB3, HBEGF, and SERPINB13. Illustrative markers for activated basal cells include IL1R2, POSTN, MMP10, and BCAM. Illustrative markers for cycling basal cells include CENPF, PTTG1, MK167, TOP2A, and STMN1. Illustrative markers for goblet cells include MUC5AC, FOXA3, CEACAM5, S100A4, PSCA, ASRGL1, LYNX1, LYPD2, KRT4, CD36, CST1, NOS2, CXCL10, IL-19, S100A8, SLC26A4, NOS2, PI3, and IDO1. Illustrative markers for club cells include MUC5B, LYPD2, SCGB3A1, SCGB1A1, MSMB, CXCL6, TFF3, BPIFB1, and TSPAN8. Illustrative markers for ciliated cells include RP11-356K23.1, RRAD, HYDIN, APOD, TSPAN19, FAM183A, PIFO, DNAH12, ERICH3, C20orf85, LRRIQ1, FGF14, C9orf135, SNTN, FXYD1, C2orf40, FOXJ1, C11orf88, PROS1, GSTA2, DYNLRB2, OMG, C1orf194, DNAAF1, KCTD12, TMEM190, and RSPH1. Illustrative markers for tuft cells include DCLK1 and ASCL2.

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

A subset of 61 genes are known to be associated with lung aveolus development: Abca12, Ace, Ada, Asxl1, Atp7a, Atxn1, Atxn11, Bmp4, Bmpr2, Cic, Creb1, Edn2, Errfi1, Fgf10, Fgfr2, Fgfr3, Fgfr4, Flt4, Foxa1, Foxf1, Foxp2, Gata6, Gh, Hopx, Hoxa5, Hoxa5, Hs6st1, Igf1, Igf1, Igfbp5, Kdr, Lif, Ltbp3, Man1a2, Man2a1, Mapk8ip3, Meg3, Myocd, Nkx2-1, Pdgfa, Pdpn, Pgr, Phf14, Pkdcc, Psen2, Ptges3, Pthlh, Rc3h2, Selenon, Sfta3-ps, Sftpd, Slc7a11, Smpd3, Sox2, Stk40, Stra6, Tcf21, Tgfb3, Tmem38b, Tmtc3, Vegfa, and Zfp157. A subset of 51 genes are known to be associated with lung epithelium development: Adamtsl2, Agr2, Aimp2, AscI1, Bmp4, Cdc42, Creb1, Creb1, Errfi1, Eya1, Fgf7, Fgf10, Fgfr2, Fgfr3, Fgfr4, Fndc3b, Foxa1, Foxa1, Foxa2, Foxj1, Foxp1, Foxp1, Foxp1, Foxp2, Foxp4, Gata6, Gpsm2, Grh12, Hmga2, Hoxa5, Igf1, Insc, KIf2, Map2k1, Map2k2, Ncor2, Nfib, Nkx2-1, Numa1, NUMB, Ppp3r1, Rbpj, Rcn3, Sav1, Shh, Sox9, Spdef, Srsf6, Thra, Thrb, Tmem38b, Wnt2, Wnt7b, Yap1. A subset of 34 genes are known to be associated with lung cell differentiation: Agr2, Aimp2, AscI1, Creb1, Ctnnb1, Eya1, Fgf10, Fndc3b, Foxa1, Foxa2, Foxj1, Foxp1, Foxp4, Gata6, Gpsm2, Grh12, Hoxa5, Igf1, Igf1, Insc, KIf2, Ncor2, Nfib, Nkx2-1, Numa1, Ppp3r1, Rbpj, Sav1, Sox9, Spdef, Thra, Thrb, Tmem38b, Yap1. A subset of 6 genes are known to be associated with lobar bronchus epithelium development: ADAMTS-like 2 (Adantsl2), anterior gradient 2 (Agr2), forkhead box p1 (Foxp1), forkhead boxp4 (FoxP4), homeobox A5 (Hoxa5), and SAM pointed domanin containing ets transcription factor (Spdef). A subset of 5 genes are known to be associated with lung goblet cell differentiation: Agr2, Foxp1, Foxp4, Hoxa5 and Spdef. The genes Foxp1 and Foxp4 are associated with negative regulation of lung goblet cell differentiation. The genes Fcxi and Nfib are associated with lung ciliated cell differentiation. Gene Ascl1 is associated with lung neuroendocrine cell differentiation. These genes are complementary to the genes provided in Table 3 below and, in some embodiments, they can be combined with the genes listed in Table 3.

Genes that are differentially expressed and positively associated with the regulation of the differentiation of airway epithelial secretory cell lineage are listed in Table 3.

| | | Gene | Gene | Network |
|---|---|---|---|---|
| | Gene | EntrezID | Directionality | Module |
| 0 | PLP2 | 5355 | down | 0 |
| 1 | GAPDH | 2597 | down | 0 |
| 2 | SNCA | 6622 | down | 0 |
| 3 | CDH3 | 1001 | down | 0 |
| 4 | FKBP4 | 2288 | down | 0 |
| 5 | CAMSAP2 | 23271 | down | 0 |
| 6 | PPP1R13B | 23368 | down | 0 |
| 7 | NISCH | 11188 | down | 0 |
| 8 | HTRA1 | 5654 | down | 0 |
| 9 | ATP11B | 23200 | down | 0 |
| 10 | ETS1 | 2113 | down | 0 |
| 11 | CPSF4 | 10898 | down | 0 |
| 12 | TLE1 | 7088 | down | 0 |
| 13 | CDK2 | 1017 | down | 0 |
| 14 | SESN1 | 27244 | down | 0 |
| 15 | GRB7 | 2886 | down | 0 |
| 16 | CERK | 64781 | down | 0 |
| 17 | ZNF318 | 24149 | down | 0 |
| 18 | MYC | 4609 | down | 1 |
| 19 | ELOVL6 | 79071 | down | 1 |
| 20 | STAMBP | 10617 | down | 1 |
| 21 | EBNA1BP2 | 10969 | down | 1 |
| 22 | MSH6 | 2956 | down | 1 |
| 23 | FAH | 2184 | down | 1 |
| 24 | EIF4EBP1 | 1978 | down | 1 |
| 25 | SLC35F2 | 54733 | down | 1 |
| 26 | RRP1B | 23076 | down | 1 |
| 27 | G3BP1 | 10146 | down | 1 |
| 28 | UTP14A | 10813 | down | 1 |
| 29 | DUSP3 | 1845 | down | 1 |
| 30 | FHL2 | 2274 | down | 3 |
| 31 | VPS72 | 6944 | down | 3 |
| 32 | ARL4C | 10123 | down | 3 |
| 33 | ARPP19 | 10776 | down | 3 |
| 34 | CDKN1B | 1027 | down | 3 |
| 35 | TP53 | 7157 | down | 3 |
| 36 | CRYZ | 1429 | down | 3 |
| 37 | PLOD3 | 8985 | down | 3 |
| 38 | DDIT4 | 54541 | down | 3 |
| 39 | LAMA3 | 3909 | down | 4 |
| 40 | INPP1 | 3628 | down | 4 |
| 41 | CDK7 | 1022 | down | 4 |
| 42 | KLHL21 | 9903 | down | 4 |
| 43 | TIAM1 | 7074 | down | 4 |
| 44 | TIPARP | 25976 | down | 4 |
| 45 | FOXJ3 | 22887 | down | 4 |
| 46 | NPC1 | 4864 | down | 4 |
| 47 | TUBB6 | 84617 | down | 5 |
| 48 | TPM1 | 7168 | down | 5 |
| 49 | RPA3 | 6119 | down | 5 |
| 50 | SFN | 2810 | down | 5 |
| 51 | ST3GAL5 | 8869 | down | 5 |
| 52 | GMNN | 51053 | down | 5 |
| 53 | ACOT9 | 23597 | down | 5 |
| 54 | BLMH | 642 | down | 6 |
| 55 | NNT | 23530 | down | 6 |
| 56 | USP1 | 7398 | down | 6 |
| 57 | FKBP14 | 55033 | down | 6 |
| 58 | HSPB1 | 3315 | down | 6 |
| 59 | TBP | 6908 | down | 6 |
| 60 | EPB41L2 | 2037 | down | 6 |
| 61 | CDCA4 | 55038 | down | 7 |
| 62 | TRAM2 | 9697 | down | 7 |
| 63 | CETN3 | 1070 | down | 7 |
| 64 | METRN | 79006 | down | 7 |
| 65 | PDLIM1 | 9124 | down | 7 |
| 66 | BRCA1 | 672 | down | 7 |
| 67 | LOXL1 | 4016 | down | 7 |

In Table 3 and associated embodiments:
“Gene ID”: at the time of filing the present disclosure, the World Wide Web at ncbi.nlm.nih.gov/gene provides a description of and the nucleic acid sequence for each GeneID listed in Table 3; the contents of each of which is incorporated herein by reference in its entirety.
“Down” indicates a gene for which a decrease in expression and/or activity in the progenitor cell is associated with the gene signature.
A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition-such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 3, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 3. In certain embodiments, a perturbation activates multiple network modules for the desired cell state transition, such as 2, 3, 4, 5, 6, 7, or more modules.

In some embodiments, one or more genes of network module 0 are modulated. In some embodiments, the present disclosure relates to the activation of network module 0, e.g., one or more of (inclusive of all of) PLP2, GAPDH, SNCA, CDH3, FKBP4, CAMSAP2, PPP1R13B, NISCH, HTRA1, ATP11B, ETS1, CPSF4, TLE1, CDK2, SESN1, GRB7, CERK, and ZNF318.

In some embodiments, one or more genes of network module 1 are modulated. In some embodiments, the present disclosure relates to the activation of network module 1, e.g., one or more of (inclusive of all of) MYC, ELOVL6, STAMBP, EBNA1BP2, MSH6, FAH, EIF4EBP1, SLC35F2, RRP1B, G3BP1, UTP14A, and DUSP3.

In some embodiments, one or more genes of network module 3 are modulated. In some embodiments, the present disclosure relates to the activation of network module 3, e.g., one or more of (inclusive of all of) FHL2, VPS72, ARL4C, ARPP19, CDKN1B, TP53, CRYZ, PLOD3, and DDIT4.

In some embodiments, one or more genes of network module 4 are modulated. In some embodiments, the present disclosure relates to the activation of network module 4, e.g., one or more of (inclusive of all of) LAMA3, INPP1, CDK7, KLHL21, TIAM1, TIPARP, FOXJ3, and NPC1.

In some embodiments, one or more genes of network module 5 are modulated. In some embodiments, the present disclosure relates to the activation of network module 5, e.g., one or more of (inclusive of all of) TUBB6, TPM1, RPA3, SFN, ST3GAL5, GMNN, and ACOT9.

In some embodiments, one or more genes of network module 6 are modulated. In some embodiments, the present disclosure relates to the activation of network module 6, e.g., one or more of (inclusive of all of) BLMH, NNT, USP1, FKBP14, HSPB1, TBP, and EPB41L2.

In some embodiments, one or more genes of network module 7 are modulated. In some embodiments, the present disclosure relates to the activation of network module 7, e.g., one or more of (inclusive of all of) CDCA4, TRAM2, CETN3, METRN, PDLIM1, BRCA1, and LOXL1.

In some embodiments, the present methods alter a gene signature in the sample of cells, comprising an activation of a network module designated in the network module column of Table 3.

In some embodiments, the activation of the network module designated in the network module column of Table 3 comprises modulating expression and/or activity of 2 or more genes within a network module.

In some embodiments, the activation of the network module designated in the network module column of Table 3 comprises modulating expression and/or activity of all of the genes within a network module.

In some embodiments, the activation of the network module designated in the network module column of Table 3 comprises modulating expression and/or activity of 2 or more genes within 2 or more network modules. In some embodiments, the activation of the network module designated in the network module column of Table 3 comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more, or 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, 67 or more, or 68 or more genes) within 2 or more network modules (e.g. 2 or more, or 3 or more, or 4 or more, or 5 or more, or 6 or more, or 7 or more network modules).

At the time of filing the present disclosure, the World Wide Web at ncbi.nlm.nih.gov/gene provides a description of and the nucleic acid sequence for each Gene listed in the genes designated as a “down” gene in the gene directionality column of Table 3; the contents of each of which is incorporated herein by reference in its entirety.

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of promoting goblet cell lineage differentiation are listed in Table 4.

TABLE 4
Perturbagens

| | | Molecular | |
|---|---|---|---|
| Perturbagen | Molecular | weight | |
| No. | formula | (g/mol) | Dose |
| 1 | C₂₃H₂₆IN₇O₂S | 591.5 | |
| 2 | C₂₄H₂₁ClF₃NO₄S | 511.9 | |
| 3 | C₂₇H₄₄O₃ | 416.6 | 10 μM |
| 4 | C₂₇H₄₀O₃ | 412.6 | 1 μM |
| 5 | C₃₀H₂₈N₆O₆S₄ | 696.8 | |
| 6 | C₃₀H₄₆O₃ | 454.7 | 1 μM |
| 7 | C₂₇H₄₄O₃ | 416.6 | 1 μM |
| 8 | C₂₇H₄₄O₂ | 400.6 | 1 μM |
| 9 | C₂₆H₄₂O₄ | 418.6 | 1 μM |
| 10 | C₂₇H₄₄O₃ | 416.6 | 1 μM |
| 11 | C₂₈H₄₄O₃ | 428.6 | 1 μM |
| 12 | C₂₇H₃₆N₆O₃S | 524.7 | |
| 13 | C₃₀H₄₆O₃ | 454.7 | |

In various embodiments herein, a perturbagen encompasses the perturbagens named in Table 4. Thus, the named perturbagens of Table 4 represent examples of perturbagens of the present disclosure.

In Table 4, the effective in vitro concentration is the concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In some embodiments, a perturbagen used in the present disclosure is a variant of a perturbagen of Table 4. A variant may be a derivative, analog, enantiomer or a mixture of enantiomers thereof or a pharmaceutically acceptable salt, solvate, hydrate, co-crystal, clathrate, or polymorph of the perturbagen of Table 4. A variant of a perturbagen of Table 4 retains the biological activity of the perturbagen of Table 4.

In some embodiments, the at least one perturbagen selected from Table 4, or a variant thereof, comprises at least 2, at least 3, at least 4, at least 5, at least 6, at least 7, at least 8, at least 9, at least 10, at least 11, at least 12, or 13 perturbagens selected from Table 4, or variants thereof.

### Methods and Perturbagens for Directing a Change in Cell State

The present disclosure relates to the restoring the abnormal numbers, ratios or bodily distributions of goblet cells, ciliated cells, or immediate progenitors thereof with respect to each other. Restoring differentiation to mucus-clearing ciliated cells while simultaneously suppressing differentiation to mucus-secreting goblet cells thereby to reduce total mucus production, which would furthermore ameliorate chronic infections and inflammation.

It has become increasingly recognized that dysfunction in the microenvironment of a stem cell niche may contribute to the morbidity of disease such as cystic fibrosis characterized by significant airway glandular remodeling in response to lung infection, with alterations including glandular hypertrophy (expansion of gland mass in existing glands) and metaplasia (replacement of one cell phenotype with another; e.g., serous to mucous cell) (Crystal et al., Proceedings of the American Thoracic Society, 2008, pp. 772-777).

Towards that, particular cellular changes in cell state can be matched to differential gene expression (which collectively define a gene signature), caused by exposure of a cell to a perturbagen. In some embodiments, the expansion of stem cells and/or progenitor cells and the formation of differentiated epithelial cells was measured by positive expression of the protein markers including Trp63, Krt5, Krt8, Scgb1a1, Atpv1b1, foxj1, Muc5ac, Cyp2f2, Cgrp, acetyl α-tubulin, and by AB/PAS+ staining for total mucus. In some embodiments, the goblet cells are marked by expression of protein Muc5ac, Foxa3, and positive AB/PAS staining. In some embodiments, the ciliated cells are marked by expression of protein Foxj1 and acetyl α-tubulin⁺. In some embodiments, the neuroendocrine cells are marked by expression of protein Cgrp+. In some embodiments, the club cells are marked by Scgb1a1⁺Cyp2f2⁺. In some embodiments, the variant club cells are marked by Scgb1a1⁺Cyp2f2⁻. In some embodiments, the expansion of the basal cells, and/or basal luminal precursor cells was measured by positive expression of the protein markers. In some embodiments, the basal cells are marked by Trp63⁺Krt5⁺. In some embodiments, the basal cells are marked by Krt14⁺. In some embodiments, the basal luminal precursor cells are marked by Trp63⁺Krt5⁺Krt8⁺.

In some embodiments, a change in cell state may be from one progenitor cell type to another progenitor cell type. For example, basal cells (Trp63⁻Krt5⁺) may change to N2ICD⁺ cell and c-myb⁺ cell. In some embodiments, a change in cell state may be from an upstream progenitor cell (e.g. a basal cell (Trp63⁻Krt5⁺)) to a downstream progenitor cell (e.g., basal luminal precursor cell (Trp63⁻Krt5⁺Krt8⁺), N2ICD⁺ cell, or c-myb⁺ cell). In some embodiments, a change in cell state may be from a progenitor cell to a differentiated cell, for example, basal luminal precursor cells (Trp63⁻Krt5⁺Krt8⁺) may change to club cells (Scgb1a1⁺Cyp2f2⁺), goblet cells (Muc5ac⁺, AB/PAS⁺), ciliated cells (acetyl α-tubulin⁺), or neuroendocrine cell (Cgrp⁺) depending on local signals; variant club cells (Scgb1a1⁺Cyp2f2⁻) to club cell (Scgb1a1⁺Cyp2f2⁺); basal cells (Krt14⁺) to secretory and ciliated cells; N21CD⁺ cell to mature secretory cells; c-myb⁺ cell to ciliated cells). In some embodiments, a change in cell state may be from one differentiated cell into another differentiated cell (e.g., Scgb1a1+ secretory cell to goblet cell (Muc5ac⁺, AB/PAS⁺), club cell to goblet cell, ciliated cell to goblet cell, club cell to ciliated cell; neuroendocrine cell (Cgrp⁺) to club cell; club cell to alveolar type II cell (LysM⁺); or club cell to alveolar type I cell (Hopx⁺)). In embodiments, a change in cell state may be from the final differentiated cell into a dedifferentiated cell (e.g. Scgb1a1⁺ secretory cells dedifferentiated into Trp63⁻Krt5⁺ basal cells).

In embodiments, certain markers are associated with certain cell types, e.g. goblet: Muc5ac, Foxa3; ciliated: Acetyl α-Tubulin, Foxj1; basal: Krt5, p63; and club: Scgb1a1.

For details on the goblet cell lineage, see, e.g., Schilders et al. Respiratory Research (2016) 17:44, the entire contents of which are incorporated by reference.

An aspect of the present disclosure is related to a method for inhibiting a change in cell state of a progenitor cell. This method includes a step of contacting a population of cells comprising a progenitor cell with at least one perturbagen selected from Table 4, or a variant of perturbagens described in Table 4. In this aspect, the at least one perturbagen is capable of altering a gene signature in the progenitor cell. In one embodiment, the progenitor cell is a basal cell.

Another aspect of the present disclosure is related to a method for inhibiting a change in cell state of a progenitor cell. This method includes a step of contacting a population of cells comprising a progenitor cell with at least one perturbagen capable of altering a gene signature in the progenitor cell. In this aspect, altering the gene signature comprises a decreased expression and/or activity in the progenitor cell of one or more genes designated as a “down” gene in the gene directionality column of Table 3. In one embodiment, the progenitor cell is a basal cell.

Yet another aspect of the present disclosure is related to a method for inhibiting a change in cell state of a progenitor cell. This method includes a step of contacting a population of cells comprising a progenitor cell with at least one perturbagen selected from Table 4, or a variant thereof, and capable of altering a gene signature in the progenitor cell.

In this aspect, altering the gene signature comprises a decreased expression and/or activity in the progenitor cell of one or more genes designated as a “down” gene in the gene directionality column of Table 3. In one embodiment, the progenitor cell is a basal cell.

In some embodiments, altering the gene signature comprises an activation of a network module designated in the network module column of Table 3. In this aspect, the activation of the network module designated in the network module column of Table 3 comprises modulating expression and/or activity of 2 or more genes within a network module. In some embodiments, the activation of the network module designated in the network module column of Table 3 comprises modulating expression and/or activity of all of the genes within a network module. In other embodiments, the activation of the network module designated in the network module column of Table 3 comprises modulating expression and/or activity of 2 or more genes within 2 or more network modules.

In some embodiments, inhibiting the change in cell state provides i) a decrease in the number of goblet cells; ii) an increase in the number of club cells; and/or iii) an increase in the number of ciliated cells. In some embodiments, inhibiting the change in cell state provides a decrease in the number of goblet cells. In some embodiments, inhibiting the change in cell state provides an increase in the number of club cells. In some embodiments, inhibiting the change in cell state provides an increase in the number of ciliated cells. In some embodiments, inhibiting the change in cell state provides an increase in the number of ciliated cells and a decrease in the number of goblet cells.

In some embodiments, inhibiting the change in cell state provides a decrease in the number of goblet cells relative to the number of basal cells obtained from a population of progenitor cells i) that is not contacted with the at least one perturbagen or ii) prior to contacting with the at least one perturbagen. In some embodiments, inhibiting the change in cell state provides a decrease in the number of goblet cells relative to the number of basal luminal precursor cells obtained from a population of progenitor cells i) that is not contacted with the at least one perturbagen or ii) prior to contacting with the at least one perturbagen. In some embodiments, inhibiting the change in cell state provides a decrease in the number of goblet cells relative to the number of club cells obtained from a population of progenitor cells i) that is not contacted with the at least one perturbagen or ii) prior to contacting with the at least one perturbagen. In some embodiments, inhibiting the change in cell state provides a decrease in the number of goblet cells relative to the number of ciliated cells obtained from a population of progenitor cells i) that is not contacted with the at least one perturbagen or ii) prior to contacting with the at least one perturbagen. In some embodiments, inhibiting the change in cell state provides a decrease in the number of goblet cells relative to the number of neuroendocrine cells obtained from a population of progenitor cells i) that is not contacted with the at least one perturbagen or ii) prior to contacting with the at least one perturbagen. In some embodiments, inhibiting the change in cell state provides an increase in the number of club cells relative to the number of basal cells, goblet cells, basal luminal precursor cells, ciliated cells, and/or neuroendocrine cells obtained from a population of progenitor cells i) that is not contacted with the at least one perturbagen or ii) prior to contacting with the at least one perturbagen. In some embodiments, inhibiting the change in cell state provides an increase in the number of ciliated cells relative to the number of basal cells, goblet cells, basal luminal precursor cells, club cells, and/or neuroendocrine cells obtained from a population of progenitor cells i) that is not contacted with the at least one perturbagen or ii) prior to contacting with the at least one perturbagen.

In some embodiments, inhibiting the change in cell state does not provide a substantial increase in the number of goblet cells.

In some embodiments, inhibiting the change in cell state provides a decrease in the ratio of the number of goblet cells to the number of basal cells relative to the ratio obtained from a population of progenitor cells that is not contacted with the at least one perturbagen. In other embodiments, inhibiting the change in cell state provides a decrease in the ratio of the number of goblet cells to the number of basal cells relative to the ratio in the population of progenitor cells prior to contacting with the at least one perturbagen.

In some embodiments, inhibiting the change in cell state provides a decrease in the ratio of the number of goblet cells to the number of basal luminal precursor cells relative to the ratio obtained from a population of progenitor cells that is not contacted with the at least one perturbagen. In some embodiments, inhibiting the change in cell state provides a decrease in the ratio of the number of goblet cells to the number of basal cells relative to the ratio obtained from a population of progenitor cells that is not contacted with the at least one perturbagen. In other embodiments, inhibiting the change in cell state provides a decrease in the ratio of the number of goblet cells to the number of basal luminal precursor cells relative to the ratio in the population of progenitor cells prior to contacting with the at least one perturbagen. In some embodiments, inhibiting the change in cell state provides a decrease in the ratio of the number of goblet cells to the number of basal cells relative to the ratio in the population of progenitor cells prior to contacting with the at least one perturbagen.

In some embodiments, inhibiting the change in cell state provides a decrease in the ratio of the number of goblet cells to the number of club cells relative to the ratio obtained from a population of progenitor cells that is not contacted with the at least one perturbagen. In other embodiments, inhibiting the change in cell state provides a decrease in the ratio of the number of goblet cells to the number of club cells relative to the ratio in the population of progenitor cells prior to contacting with the at least one perturbagen.

In some embodiments, inhibiting the change in cell state provides a decrease in the ratio of the number of goblet cells to the number of ciliated cells relative to the ratio obtained from a population of progenitor cells that is not contacted with the at least one perturbagen. In other embodiments, inhibiting the change in cell state provides a decrease in the ratio of the number of goblet cells to the number of ciliated cells relative to the ratio in the population of progenitor cells prior to contacting with the at least one perturbagen.

In some embodiments, inhibiting the change in cell state provides a decrease in the ratio of the number of goblet cells to the number of neuroendocrine cells relative to the ratio obtained from a population of progenitor cells that is not contacted with the at least one perturbagen. In other embodiments, inhibiting the change in cell state provides a decrease in the ratio of the number of goblet cells to the number of neuroendocrine cells relative to the ratio in the population of progenitor cells prior to contacting with the at least one perturbagen.

In some embodiments, inhibiting the change in cell state provides an increase in the ratio of the number of club cells to the number of basal cells relative to the ratio obtained from a population of progenitor cells that is not contacted with the at least one perturbagen. In other embodiments, inhibiting the change in cell state provides an increase in the ratio of the number of club cells to the number of basal cells relative to the ratio in the population of progenitor cells prior to contacting with the at least one perturbagen.

In some embodiments, inhibiting the change in cell state provides an increase in the ratio of the number of club cells to the number of basal luminal precursor cells relative to the ratio obtained from a population of progenitor cells that is not contacted with the at least one perturbagen. In other embodiments, inhibiting the change in cell state provides an increase in the ratio of the number of club cells to the number of basal luminal precursor cells relative to the ratio in the population of progenitor cells prior to contacting with the at least one perturbagen. In some embodiments, inhibiting the change in cell state provides an increase in the ratio of the number of club cells to the number of basal cells relative to the ratio obtained from a population of progenitor cells that is not contacted with the at least one perturbagen. In other embodiments, inhibiting the change in cell state provides an increase in the ratio of the number of club cells to the number of basal cells relative to the ratio in the population of progenitor cells prior to contacting with the at least one perturbagen.

In some embodiments, inhibiting the change in cell state provides an increase in the ratio of the number of club cells to the number of ciliated cells relative to the ratio obtained from a population of progenitor cells that is not contacted with the at least one perturbagen. In other embodiments, inhibiting the change in cell state provides an increase in the ratio of the number of club cells to the number of ciliated cells relative to the ratio in the population of progenitor cells prior to contacting with the at least one perturbagen.

In some embodiments, inhibiting the change in cell state provides an increase in the ratio of the number of club cells to the number of neuroendocrine cells relative to the ratio obtained from a population of progenitor cells that is not contacted with the at least one perturbagen. In other embodiments, inhibiting the change in cell state provides an increase in the ratio of the number of club cells to the number of neuroendocrine cells relative to the ratio in the population of progenitor cells prior to contacting with the at least one perturbagen.

In some embodiments, inhibiting the change in cell state provides an increase in the ratio of the number of ciliated cells to the number of basal luminal precursor cells relative to the ratio obtained from a population of progenitor cells that is not contacted with the at least one perturbagen. In other embodiments, inhibiting the change in cell state provides an increase in the ratio of the number of ciliated cells to the number of basal luminal precursor cells relative to the ratio in the population of progenitor cells prior to contacting with the at least one perturbagen. In some embodiments, inhibiting the change in cell state provides an increase in the ratio of the number of ciliated cells to the number of basal cells relative to the ratio obtained from a population of progenitor cells that is not contacted with the at least one perturbagen.

In other embodiments, inhibiting the change in cell state provides an increase in the ratio of the number of ciliated cells to the number of basal cells relative to the ratio in the population of progenitor cells prior to contacting with the at least one perturbagen.

In some embodiments, inhibiting the change in cell state provides an increase in the ratio of the number of ciliated cells to the number of goblet cells relative to the ratio obtained from a population of progenitor cells that is not contacted with the at least one perturbagen. In other embodiments, inhibiting the change in cell state provides an increase in the ratio of the number of ciliated cells to the number of goblet cells relative to the ratio in the population of progenitor cells prior to contacting with the at least one perturbagen.

In some embodiments, inhibiting the change in cell state does not provide a substantial increase in the number of goblet cells.

In some embodiments, inhibiting the change in cell state provides a decrease in the number of goblet cells due in part to decreased cell proliferation of the goblet cells, basal cells, basal luminal precursor cells, and/or club cells. In some embodiments, inhibiting the change in cell state provides a decrease in the number of goblet cells due in part to a decreased lifespan of the goblet cells, basal cells, basal luminal precursor cells, and/or club cells. In some embodiments, inhibiting the change in cell state provides a decrease in the number of goblet cells due in part to increased cell death among the goblet cells, basal cells, basal luminal precursor cells, and/or club cells. In some embodiments, inhibiting the change in cell state provides a decrease in the number of goblet cells due in part to blocking the progression from: i) basal cell to basal luminal precursor cell; ii) basal luminal precursor cell to club cell; and/or iii) club cell to goblet cell.

In some embodiments, the number of basal cells is increased. In this aspect, the increase in the number of basal cells is due in part to i) increased cell proliferation of the basal cells; ii) an increased lifespan of the basal cells; and/or iii) decreased cell death among the basal cells. In another aspect, the increase in the number of basal cells is relative to the number of basal cells in a population of basal cells that is not contacted with the at least one perturbagen. In another aspect, the increase in the number of basal cells is relative to the number of basal cells in the population prior to contacting with the at least one perturbagen. In yet another aspect, the increase in the number of basal cells is due to inhibiting a change of cell state from a basal cell into the goblet cell lineage and/or club cell lineage.

In some embodiments, the number of goblet cells is decreased after contacting the population of cells comprising a basal cell with the at least one perturbagen. In this aspect, inhibiting the change in cell state provides a decrease in the ratio of the number of goblet cells to the number of basal cells relative to the ratio obtained from a population of progenitor cells that is not contacted with the at least one perturbagen. In another aspect, inhibiting the change in cell state provides a decrease in the ratio of the number of goblet cells to the number of basal cells relative to the ratio in the population of progenitor cells prior to contacting with the at least one perturbagen.

Methods for determining the extension of the lifespan of a specific cell type or a reduction of cell death is well known in the art. For examples, markers for dying cells, e.g., caspases can be detected, or dyes for dead cells, e.g., methylene blue, may be used. Methods for counting cells are well known in the art. Non-limiting examples include hemocytometry, flow cytometry, and cell sorting techniques, e.g., fluorescence activated cell sorting (FACS).

In some embodiments, for any herein described method, the at least one perturbagen selected from Table 4, or a variant thereof, comprises at least 2, at least 3, at least 4, at least 5, at least 6, at least 7, at least 8, at least 9, at least 10, at least 11, at least 12, or 13 perturbagens selected from Table 4, or variants thereof.

In some embodiments, for any herein described method, at least one perturbagen selected from Table 4.

In some embodiments, altering the gene signature comprises decreased expression and/or decreased activity in the progenitor cell of one or more genes designated as a “down” gene in the gene directionality column of Table 3. In this aspect, the one or more genes selected from Table 3 comprises 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more, or 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, 67 or more, or 68 or more genes designated as a “down” gene in the gene directionality column of Table 3. In some embodiments, the one or more genes designated as a “down” gene in the gene directionality column of Table 3 are selected from PLP2, GAPDH, SNCA, CDH3, FKBP4, CAMSAP2, PPP1R13B, NISCH, HTRA1, ATP11B, ETS1, CPSF4, TLE1, CDK2, SESN1, GRB7, CERK, ZNF318, MYC, ELOVL6, STAMBP, EBNA1BP2, MSH6, FAH, EIF4EBP1, SLC35F2, RRP1B, G3BP1, UTP14A, DUSP3, FHL2, VPS72, ARL4C, ARPP19, CDKN1B, TP53, CRYZ, PLOD3, DDIT4, LAMA3, INPP1, CDK7, KLHL21, TIAM1, TIPARP, FOXJ3, NPC1, TUBB6, TPM1, RPA3, SFN, ST3GAL5, GMNN, ACOT9, BLMH, NNT, USP1, FKBP14, HSPB1, TBP, EPB41L2, CDCA4, TRAM2, CETN3, ETRN, PDLIM1, BRCA1, and LOXL1.

In some embodiments, for any herein described method, contacting the population of cells comprising a progenitor cell occurs in vitro or ex vivo. In some embodiments, for any herein described method, contacting the population of cells comprising a progenitor cell occurs in vivo in a subject. In this aspect, the subject is a human. In some embodiments, the human is an adult human.

An aspect of the present disclosure is related to a perturbagen for use in any herein described method.

An aspect of the present disclosure is related to a pharmaceutical composition containing the perturbagen used in any herein described method.

An aspect of the present disclosure is related to a method for inhibiting the formation of a goblet cell or an immediate progenitor thereof. This method includes a step of exposing a starting population of progenitor cells comprising at least one basal cell to a perturbation having a perturbation signature that prevents progression of a progenitor cell into and/or reduces the likelihood that a progenitor cell will progress into a goblet cell or other lineage associated progenitor thereof.

In this aspect, the perturbation signature comprises a decreased expression and/or activity in the progenitor cells of one or more genes selected from Table 3 designated as a “down” gene in the gene directionality column of Table 3.

In this aspect, the activation of the network module designated in the network module column of Table 3 comprises modulating expression and/or activity of 2 or more genes within 2 or more network modules.

In some embodiments, for any herein described method, the formation of goblet cells is inhibited.

In some embodiments, for any herein described method, the perturbation signature comprises an activation of a network module designated in the network module column of Table 3. In this aspect, the activation of the network module designated in the network module column of Table 3 comprises modulating expression and/or activity of 2 or more genes within a network module. In another aspect, the activation of the network module designated in the network module column of Table 3 comprises modulating expression and/or activity of all of the genes within a network module. An aspect of the present disclosure is related to a method for promoting the formation of a ciliated cell, or an immediate progenitor thereof. This method includes a step of exposing a starting population of progenitor cells comprising at least one basal cell to a perturbation having a perturbation signature that promotes progression of a progenitor cell into and/or increases the likelihood that a progenitor cell will progress into a ciliated cell or other lineage associated progenitor thereof. In this aspect, the perturbation signature comprises a decreased expression and/or activity in the progenitor cells of one or more genes selected from designated as a “down” gene in the gene directionality column of Table 3. In this aspect, the activation of the network module designated in the network module column of Table 3 comprises modulating expression and/or activity of 2 or more genes within 2 or more network modules.

In some embodiments, for any herein described method, the perturbation signature comprises an activation of a network module designated in the network module column of Table 3. In this aspect, the activation of the network module designated in the network module column of Table 3 comprises modulating expression and/or activity of 2 or more genes within a network module. In another aspect, the activation of the network module designated in the network module column of Table 3 comprises modulating expression and/or activity of all of the genes within a network module.

In embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (e.g. including an appropriate vehicle only control such as DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (e.g. including an appropriate vehicle only control such as DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (e.g. including an appropriate vehicle only control such as DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (e.g. including an appropriate vehicle only control such as DMSO).

In some embodiments, the subject has an abnormal number of one or more of basal cells, basal luminal precursor cells, goblet cells, ciliate cells, or progenitor cells thereof, or a disease or disorder characterized thereby. In some embodiments, the subject has a reduced number of basal cells, basal luminal precursor cells, or a disease or disorder characterized thereby. In some embodiments, the subject has a reduced number of ciliated cells, or a disease or disorder characterized thereby. In some embodiments, the subject has an increased number of goblet cells or disorder characterized thereby. In some embodiments, the subject has a reduced number of ciliated cells, and an increased number of goblet cells, or a disease or disorder characterized thereby. In some embodiments, the subject has an abnormal bodily distribution of one or more of basal cells, basal luminal precursor cells, goblet cells, ciliate cells, club cells, neuroendocrine cells, or a disease or disorder characterized thereby.

Methods for evaluating abnormal numbers or bodily distribution of one or more of goblet cells, ciliate cells, club cells, neuroendocrine cells are well known in the art (See Examples 1-2 infra).

Exemplary abnormal bodily distribution of one or more of goblet cells, ciliate cells, club cells, neuroendocrine cells includes, but is not limited to, e.g., (1) an increased number of ciliated cells in the pseudostratified airway epithelium of the lung; (2) a decreased number of secretory cells such as goblet cells in the pseudostratified airway epithelium of the lung; (3) an increased number of club cells in the pseudostratified airway epithelium of the lung; (4) an increased number of neuroendocrine cells the pseudostratified airway epithelium of the lung; or a combination of any two or more thereof.

In some embodiments, the subject has an abnormal ratio of the number of goblet cells to the number of ciliated cells, the number of goblet cells to the number of club cells, the number of goblet cells to the number of club cells, and the number of goblet cells to the number of neuroendocrine cells; or a disease or disorder characterized thereby.

Additionally or alternatively, in some embodiments, the method provides an increase in expression and/or activity of Notch signaling pathway proteins including Notch 3 signaling and Grhl2 (regulating basal luminal precursor cell differentiation); Notch^{high} signaling, Notch 1 and Notch 2 (regulating club cell differentiation); or Notch^{low} signaling, Notch 1 and Notch 2 (regulating ciliated cell differentiation), optionally as compared to the expression and/or activity in the absence of a perturbagen. In some embodiments, the method provides an increase in expression and/or activity of Notch proteins, optionally as compared to the expression and/or activity prior to contacting with the at least one perturbagen. In some embodiments, the method provides a decrease in expression and/or activity of Hedgehog signaling pathway (Hh) protein (e.g., smoothened (SMO)), optionally as compared to the expression and/or activity in the absence of a perturbagen. In some embodiments, the method provides a decrease in expression and/or activity of Hh protein, optionally as compared to the expression and/or activity prior to contacting with the at least one perturbagen. In some embodiments, the method provides a decrease in expression and/or activity of the transforming growth factor-β (TGF-β) protein, optionally as compared to the expression and/or activity in the absence of a perturbagen. In some embodiments, the method provides a decrease in expression and/or activity of TGF-β protein, optionally as compared to the expression and/or activity prior to contacting with the at least one perturbagen. In some embodiments, the method provides a decrease in expression and/or activity of jagged proteins (Jag1 and Jag2), optionally as compared to the expression and/or activity in the absence of a perturbagen. In some embodiments, the method provides a decrease in expression and/or activity of Jag1 and Jag2 proteins, optionally as compared to the expression and/or activity prior to contacting with the at least one perturbagen. In some embodiments, the method provides a decrease in expression and/or activity of bone morphogenetic proteins (BMPs), optionally as compared to the expression and/or activity in the absence of a perturbagen. In some embodiments, the method provides a decrease in expression and/or activity of BMPs, optionally as compared to the expression and/or activity prior to contacting with the at least one perturbagen. In some embodiments, the method provides a decrease in expression and/or activity of Activin A protein, optionally as compared to the expression and/or activity in the absence of a perturbagen. In some embodiments, the method provides a decrease in expression and/or activity of Activin A protein, optionally as compared to the expression and/or activity prior to contacting with the at least one perturbagen.

In yet another aspect, the present disclosure provides a perturbagen for use in any herein disclosed method.

In a further aspect, the present disclosure provides a pharmaceutical composition comprising at least one perturbagen for use in any herein disclosed method.

### Methods and Perturbagens for Treating a Disease or Disorder

The term “disease or disorder” as used in context of the present disclosure refers to a disease or condition in the airway of respiratory system that is characterized by abnormal numbers, ratios or bodily distributions of goblet cells and/or ciliated cells, or immediate progenitors thereof with respect to each other. The airway epithelium plays a critical role in maintaining the conduit for air to and from the alveoli. It is central to the defenses of the lung against pathogens and particulates inhaled from the environment, with the combined function of secretory and ciliated cells maintaining efficient mucociliary clearance, and a variety of other host defense processes. Airway epithelial cells are central to the pathogenesis of major lung disorders, including chronic obstructive pulmonary disease (COPD), asthma, and bronchogenic carcinoma. In these disorders, the function of the airway epithelium is further modified by local inflammatory/immune signals.

The present disclosure relates to the treatment of diseases or disorders characterized by abnormal numbers, ratios or bodily distribution of goblet cell, ciliated cell, club cell, neuroendocrine cell, or immediate progenitors thereof with respect to each other. The ability of a perturbagen to specifically promote the formation of ciliated cell and to reduce or suppress the formation of goblet cell would be valuable in designing a therapeutic composition for such diseases of disorders. As example, for a disease characterized by an increased number of goblet cells, a therapeutic composition comprising at least one perturbagen that decreases the number of goblet cells could be beneficial, and/or a disease (including the same disease) that would benefit from decrease the number of goblet cells could be treated by a therapeutic composition comprising at least one perturbagen that decreases the number of goblet cells.

An aspect of the present disclosure is a method for promoting the formation of a ciliated cell, or an immediate progenitor thereof. This method includes the step of exposing a starting population of stem/progenitor cells comprising a non-lineage committed basal cell to a perturbation having a perturbation signature that promotes the transition of the starting population of stem/progenitor cells into a ciliated cell, wherein the perturbation signature a decreased expression and/or activity in the non-lineage committed basal cell of one or more genes selected from the genes designated as a “down” gene in the gene directionality column of Table 3. In some embodiments, the perturbation signature comprises an increase in expression and/or activity in the progenitor cell of an activation of a network module designated in the network module column of Table 3. In some embodiments, the activation of one or more genes of the network module designated in the network module column of Table 3 comprises modulating expression and/or activity of 2 or more genes within a network module. In some embodiments, the activation of one or more genes of the network module designated in the network module column of Table 3 comprises modulating expression and/or activity of 3 or more genes within a network module. In some embodiments, the activation of one or more genes of the network module designated in the network module column of Table 3 comprises modulating expression and/or activity of 5 or more genes within a network module. In some embodiments, the activation of one or more genes of the network module designated in the network module column of Table 3 comprises modulating expression and/or activity of all genes within a network module. In some embodiments, the perturbation signature comprises a decrease in expression and/or activity in the progenitor cell of one or more genes designated as a “down” gene in the gene directionality column of Table 3. In some embodiments, the perturbation signature comprises a decrease in expression and/or activity in the progenitor cell of two or more genes designated as a “down” gene in the gene directionality column of Table 3.

An aspect of the present disclosure is a method of increasing a quantity of basal luminal precursor cell (Trp63⁻Krt5⁺Krt8⁺), or immediate progenitors thereof in a subject in need thereof. The method comprises exposing a starting population of stem/progenitor cells comprising a non-lineage committed basal cell (Trp63⁻Krt5⁺) to a pharmaceutical composition that promotes the formation of lineage specific progenitor population selected from basal luminal precursor cell (Trp63⁻Krt5⁺Krt8⁺), Krt14⁺, N2ICD⁺ cell, or c-myb⁺ cell, the pharmaceutical composition promoting the transition of a primitive stem/progenitor population into the lineage specific progenitor population that has the capacity to differentiate into goblet cell, club cell, ciliated cell, neuroendocrine cell, or immediate progenitors thereof. In this aspect, the pharmaceutical composition comprises at least one perturbagen selected from Table 4, or a variant thereof.

An aspect of the present disclosure is a method for decreasing a quantity of goblet cells, or immediate progenitors thereof in a subject in need thereof. The method comprises exposing a starting population of stem/progenitor cells comprising basal cells to a pharmaceutical composition that promotes the formation of lineage specific progenitor population selected from basal luminal precursor cell (Trp63⁻Krt5⁺Krt8⁺), Krt14⁺, N21CD⁺ cell, or c-myb⁺ cell, the pharmaceutical composition promoting the transition of a primitive stem/progenitor population into the lineage specific progenitor population that has the capacity to differentiate into goblet cell, club cell, ciliated cell, neuroendocrine cell, or immediate progenitors thereof. In this aspect, the pharmaceutical composition comprises at least one perturbagen selected from Table 4, or a variant thereof.

An aspect of the present disclosure is related to a method for treating a disease or disorder characterized by an abnormal number of goblet cells or an increase in the production of mucus by goblet cells. This method includes the steps of (a) administering to a patient in need thereof a therapeutically effective amount of at least one perturbagen selected from Table 4, or a variant thereof, wherein the at least one perturbagen is capable of changing a gene signature in a progenitor cell or (b) administering to a patient in need thereof a cell, the cell having been contacted with at least one perturbagen selected from Table 4, or a variant thereof, wherein the progenitor cell is a basal cell. In this aspect, the at least one perturbagen is capable of changing a gene signature in a progenitor cell. In this aspect, the progenitor cell is a basal cell.

In some embodiments, for any herein described method, the disease or disorder is caused by an increase in the number of goblet cells or an increase in the production of mucus by goblet cells.

In some embodiments, for any herein described method, the administering is via intraosseous injection or intraosseous infusion. In some embodiments, for any herein described method, the administering the cell is via intravenous injection or intravenous infusion. In some embodiments, for any herein described method, the administering is simultaneously or sequentially to one or more mobilization agents. In some embodiments, for any herein described method, the administering of the perturbagen is via respiratory tract, oral, intravenous, intramuscular, intraperitoneal, subcutaneous, intra-articular injection, and/or infusion route.

In some embodiments, for any herein described method, the disease or disorder is caused by an increase in the number of goblet cells or an increase in the production of mucus by goblet cells. In some embodiments, for any herein described method, the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

In some embodiments, for any herein described method, the at least one perturbagen is administered on the basis of previously determining that the patient exhibits an abnormal number of goblet cells, or a disease or disorder characterized thereby. In some embodiments, for any herein described method, the at least one perturbagen is administered on the basis of previously determining that the patient exhibits an abnormal function of goblet cells, or a disease or disorder characterized thereby. In this aspect, the abnormal function of goblet cells includes increase in the production of mucus by the goblet cells.

An aspect of the present disclosure is related to a method for treating a disease or disorder characterized by an abnormal ratio of goblet cells to basal cells. This method includes the steps of a) administering to a patient in need thereof at least one perturbagen selected from Table 4, or a variant thereof, wherein the at least one perturbagen is capable of changing a gene signature in a basal cell or (b) administering to a patient in need thereof a cell, the cell having been contacted with at least one perturbagen selected from Table 4, or a variant thereof.

In some embodiments, the abnormal ratio comprises a decreased number of goblet cells and/or an increased number of basal cells. In some embodiments, the abnormal ratio comprises an increased number of basal cells. In some embodiments, the abnormal ratio comprises a decreased number of goblet cells. In some embodiments, for any herein described method, the at least one perturbagen is capable of changing a gene signature in a basal cell.

In some embodiments, for any herein described method, the administering is via intraosseous injection or intraosseous infusion. In some embodiments, for any herein described method, the administering the cell is via intravenous injection or intravenous infusion. In some embodiments, for any herein described method, the administering is simultaneously or sequentially to one or more mobilization agents. In some embodiments, for any herein described method, the administering of the perturbagen is via respiratory tract, oral, intravenous, intramuscular, intraperitoneal, subcutaneous, intra-articular injection, and/or infusion route. In another aspect, for any herein described method, for any herein described method, the at least one perturbagen is administered on the basis of previously determining that the patient exhibits the abnormal ratio of goblet cells to basal cells, or a disease or disorder characterized thereby.

In some embodiments, for any herein described method, the disease or disorder is caused by an increase in the number of goblet cells or an increase in the production of mucus by goblet cells. In some embodiments, for any herein described method, the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

In this aspect, for any herein described method, the at least one perturbagen is capable of changing a gene signature in a basal cell.

In some embodiments, the present disclosure is related to a method for selecting the patient for any herein described methods. This method includes the steps of obtaining from a subject having the disease or disorder a sample of cells comprising a basal cell; and contacting the sample of cells with least one perturbagen selected from Table 4, or a variant thereof. In this aspect, the at least one perturbagen alters a gene signature in the sample of cells.

In some embodiments, the present disclosure is related to a method for selecting the patient for any herein described methods. This method includes the steps of obtaining from a subject having the disease or disorder a sample of cells comprising a basal cell; and contacting the sample of cells with at least one perturbagen capable of altering a gene signature in a basal cell. In this aspect, the at least one perturbagen decreases in the sample of cells the expression and/or activity of one or more genes selected from Table 3 designated as a “down” gene in the gene directionality column of Table 3.

In some embodiments, the perturbation signature comprises an activation of a network module designated in the network module column of Table 3. In some embodiments, the activation of the network module designated in the network module column of Table 3 comprises modulating expression and/or activity of 2 or more genes within a network module. In this aspect, the activation of the network module designated in the network module column of Table 3 comprises modulating expression and/or activity of all of the genes within a network module. In some embodiments, the activation of the network module designated in the network module column of Table 3 comprises modulating expression and/or activity of 2 or more genes within 2 or more network modules.

In some embodiments, the present disclosure is related to a method for selecting the patient for any herein described methods. This method includes the steps of obtaining from a subject having the disease or disorder a sample of cells comprising a basal cell; and contacting the sample of cells with at least one perturbagen selected from Table 4, or a variant thereof. In this aspect, the at least one perturbagen decreases in the sample of cells the expression and/or activity of one or more genes selected from Table 3 designated as a “down” gene in the gene directionality column of Table 3.

In some embodiments, the method alters a gene signature in the sample of cells, comprising activation of a network module designated in the network module column of Table 3. In this aspect, the activation of the network module designated in the network module column of Table 3 comprises modulating expression and/or activity of 2 or more genes within a network module. In a further aspect, the activation of the network module designated in the network module column of Table 3 comprises modulating expression and/or activity of all of the genes within a network module.

In some embodiments, the activation of the network module designated in the network module column of Table 3 comprises modulating expression and/or activity of 2 or more genes within 2 or more network modules.

In some embodiments, the present disclosure is related to a method for selecting the patient for any herein described methods. This method includes the steps of obtaining from a subject having the disease or disorder a sample of cells comprising a basal cell; and contacting the sample of cells with least one perturbagen selected from Table 4, or a variant thereof. In this aspect, when the at least one perturbagen alters a gene signature in the sample of cells, the subject is selected as a patient.

In some embodiments, the present disclosure is related to a method for selecting the patient for any herein described methods. This method includes the steps of obtaining from a subject having the disease or disorder a sample of cells comprising a basal cell; and contacting the sample of cells with at least one perturbagen capable of altering a gene signature in a basal cell. In this aspect, when the at least one perturbagen decreases in the sample of cells the expression and/or activity of one or more genes selected from Table 3 designated as a “down” gene in the gene directionality column of Table 3, the subject is selected as a patient. In this aspect, the method alters a gene signature in the sample of cells, comprising activation of a network module designated in the network module column of Table 3. In some embodiments, the activation of the network module designated in the network module column of Table 3 comprises modulating expression and/or activity of 2 or more genes within a network module. In this aspect, the activation of the network module designated in the network module column of Table 3 comprises modulating expression and/or activity of all of the genes within a network module. In some embodiments, the activation of the network module designated in the network module column of Table 3 comprises modulating expression and/or activity of 2 or more genes within 2 or more network modules.

In some embodiments, the present disclosure is related to a method for selecting the patient for any herein described methods. This method includes the steps of obtaining from a subject having the disease or disorder a sample of cells comprising a basal cell; and contacting the sample of cells with at least one perturbagen selected from Table 4, or a variant thereof. In this aspect, when the at least one perturbagen decreases in the sample of cells the expression and/or activity of one or more genes selected from Table 3 designated as a “down” gene in the gene directionality column of Table 3, the subject is selected as a patient. In this aspect, the method alters a gene signature in the sample of cells, comprising activation of a network module designated in the network module column of Table 3. In this aspect, the activation of the network module designated in the network module column of Table 3 comprises modulating expression and/or activity of 2 or more genes within a network module. In a further aspect, the activation of the network module designated in the network module column of Table 3 comprises modulating expression and/or activity of all of the genes within a network module. In some embodiments, the activation of the network module designated in the network module column of Table 3 comprises modulating expression and/or activity of 2 or more genes within 2 or more network modules.

In some embodiments, the present disclosure is related to the use of the perturbagen of Table 4, or a variant thereof in the manufacture of a medicament for treating a disease or disorder characterized by an abnormal ratio of goblet cells to basal cells. Another aspect of the present disclosure is related to the use of the perturbagen of Table 4, or a variant thereof in the manufacture of a medicament for treating a disease or disorder characterized by an abnormal ratio of goblet cells to basal luminal cells, club cells, ciliated cells, and/or neuroendocrine cells. Some embodiments of the present disclosure are related to the use of the perturbagen of Table 4, or a variant thereof in the manufacture of a medicament for treating a disease or disorder characterized by an abnormal ratio of goblet cells to basal cells, club cells, ciliated cells, and/or neuroendocrine cells.

In some embodiments, the present disclosure is related to a method for identifying a candidate perturbation for promoting the transition of a starting population of basal cells into goblet cells or immediate progenitors thereof. This method includes the steps of exposing the starting population of basal cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of basal cells into goblet cells or immediate progenitors thereof following exposure of the population of cells to the perturbation; and identifying the perturbation as a candidate perturbation for promoting the transition of a population of basal cells into goblet cells or immediate progenitors thereof based on the perturbation signature. In this aspect, the perturbation signature is a decrease in expression and/or activity in the basal cell of one or more genes selected from Table 3 designated as a “down” gene in the gene directionality column of Table 3.

In some embodiments, the method alters a gene signature in the sample of cells, comprising activation of a network module designated in the network module column of Table 3. In this aspect, the activation of the network module designated in the network module column of Table 3 comprises modulating expression and/or activity of 2 or more genes within a network module. In a further aspect, the activation of the network module designated in the network module column of Table 3 comprises modulating expression and/or activity of all of the genes within a network module.

In some embodiments, the activation of the network module designated in the network module column of Table 3 comprises modulating expression and/or activity of 2 or more genes within 2 or more network modules.

In some embodiments, for any one of herein described method, the administering occurs about once per day for one or more days. In embodiments, the administering occurs more than once per day for one or more days. In some embodiments, the administering occurs at most once per day for one or more days. In some embodiments, the administering occurs substantially continuously per administration period.

In some embodiments, for any one of herein described method, the at least one perturbagen is administered to the patient via intranasal administration. In some embodiments, for any one of herein described methods, the at least one perturbagen is administered to the patient via oral inhaled administration. In some embodiments, for any one of herein described methods, the at least one perturbagen is administered to the patient via intra-tracheal instillation. In some embodiments, for any one of herein described methods, the at least one perturbagen is administered to the patient via intra-tracheal inhalation. In some embodiments, for any one of herein described methods, the at least one perturbagen is administered to the patient via aerosol administration in the pulmonary airway. In some embodiments, for any one of herein described methods, the at least one perturbagen is administered to the patient via aerosol administration in the pulmonary airway using a device selected from the group consisting of nebulizer (e.g., jet nebulizer, ultrasound nebulizer, vibrating mesh nebulizer), metered-dose inhaler, and dry powder inhaler (DPI).

In some embodiments, for any herein described method, the present disclosure provides a pharmaceutical composition comprising at least one perturbagen that decreases the differentiation to goblet cells, increases the differentiation to ciliated cells without impacting other epithelial lineages.

In some embodiments, for any herein described method, the change in cells state provides one or more of: (a) decreased secretion of mucus by a goblet cell and (b) decreased synthesis of one or more mucins, optionally selected from MUC5AC, MUC5B, MUC2, MUC4, MUC7, MUC8, or MUC19.

An aspect of the present disclosure is a method for making a therapeutic agent for a disease or disorder characterized by an abnormal ratio of goblet cells to basal luminal cells, club cells, ciliated cells, and/or neuroendocrine cells, comprising: (a) identifying a candidate perturbation according to any herein described method; and (b) formulating the candidate perturbation as a therapeutic agent for the treatment of the disease or disorder. Some embodiments of the present disclosure are related a method for making a therapeutic agent for a disease or disorder characterized by an abnormal ratio of goblet cells to basal cells, club cells, ciliated cells, and/or neuroendocrine cells, comprising: (a) identifying a candidate perturbation according to any herein described method; and (b) formulating the candidate perturbation as a therapeutic agent for the treatment of the disease or disorder.

### Disease or Disorders

Mucociliary clearance has an important innate immune function in healthy airways, with contributions by the ciliated cells in the conducting airway epithelium and the secretory cells, including goblet cells, the mucous and serous cells in the submucosal glands. Coordinated ciliary motion clears mucus-bound particles from the airways. Mucus binds infectious agents and particulates, but also has antioxidant, anti-protease, and antimicrobial properties. Expression of specific mucins is segregated in different secretory cells. For example, MUC5AC is localized to goblet cells and MUC5B is localized to mucous cells of submucosal glands in the healthy airway.

The secretory cells are implicated in lung diseases as mucus hypersecretion/overproduction, goblet cell hypertrophy and hyperplasia, and submucosal gland hypertrophy, which are common pathologic features of chronic inflammatory airway diseases including asthma, COPD, and cystic fibrosis (CF).

Distinct signaling pathways regulate goblet cell hyperplasia and differ according to the disease and the environmental insult. In asthma, IL-13 and epidermal growth factor receptor (EGFR) activate independent pathways to regulate goblet cell hyperplasia. In COPD, tobacco smoke or reactive oxygen species regulate goblet cell hyperplasia by either EGFR-dependent or EGFR-independent pathways. Overexpression of the epithelial sodium channel creates cystic fibrosis-like primary defects of increased sodium absorption and diminished airway surface liquid volume, resulting in goblet cell metaplasia and mucus obstruction of airways. A profile of secretory cell positive and negative regulatory transcription factors is emerging with FoxA2, a negative regulatory factor for goblet cell metaplasia; and catenin and SAM pointed domain containing ETS transcription factor [SPDEF], positive regulatory factors for goblet cell metaplasia.

### Chronic Obstructive Pulmonary Disease (COPD)

The small airway epithelium is a critical zone for several common lung diseases including chronic obstructive pulmonary disease (COPD). COPD is a slowly progressing disease with a long asymptomatic phase, during which lung function continues to decline. The prevalence of COPD, characterized by an irreversible limitation of expiratory airflow, is growing in the United States and worldwide, and no cure is available. Cigarette smoking or exposure to noxious agents induces an inflammatory process in the lungs and airways of the bronchial tree that leads to small airway disease and parenchymal destruction. Loss of elasticity of the alveolar attachments, or their destruction, is a hallmark of emphysema. The inability of the lungs to empty results in air trapping and hyperinflation, manifested as dyspnea on exertion. Chronic bronchitis is a common form of chronic obstructive pulmonary disease (COPD) associated with persistent inflammation and pathogen colonization of the airway epithelium.

Upregulation of antioxidant-related genes in the airway epithelium in smokers has been identified as being associated with the development of chronic bronchitis, for example, genes associated with glutathione metabolism including GPX2 (glutathione peroxidase 2), GCLM (glutamate-cysteine ligase modifier subunit), GSR (glutathione-disulfide reductase), GCLC (glutamate-cysteine ligase catalytic subunit), GPX3 (glutathione peroxidase 3), IDH2 (isocitrate dehydrogenase (NADP(+)) 2), GSTA2 (glutathione S-transferase alpha 2); genes associated with redox balance including ADH7 (alcohol dehydrogenase 7), AKR1C3 (aldo-keto reductase family 1 member C3), AKR1B1 (aldo-keto reductase family 1 member B), TXNRD1 (thioredoxin reductase 1); genes associated with pentose phosphate including TALDO1 (transaldolase 1), PGD (phosphogluconate dehydrogenase), TKT (transketolase), G6PD (glucose-6-phosphate dehydrogenase), ALDOA (aldolase, fructose-bisphosphate A) (See Hackett et al. supra). (Hackett et al., Am. J.

Respiratory Cell and Mol. Biology, 2002, pp. 331-343) The currently available medications for preventing and relieving symptoms of COPD include: inhaled β2-agonist such as levalbuterol, albuterol, formoterol, foradil, salmeterol, arformoterol; inhaled anticholinergics such as ipratropium, triotropium; inhaled corticosteroids such as beclomethasone, budesonide, fluticasone, triamcinolone; salmeterol and fluticasone; formoterol and budesonide; and theophylline.

None of the currently available COPD medication has been shown to alter the progressive deterioration of lung function that characterizes the disease. It has been reported that jagged inhibition with antibodies results in an increase in ciliated cells at the expense of club cells, and reverses the goblet cell hyperplasia, which is potentially important in COPD patients to reduce the mucus production and to increase the clearance by the ciliated cells (Schilders et al., Respiratory Research, 2016, vol. 17, pp. 1-17).

In some embodiments, the present disclosure provides the use of perturbagen to probe the molecular mechanisms of goblet cell degranulation by assaying the genes expressions and or gene signature induced by the perturbagen selected from Table 4. The gene signature discovered by the present disclosure provides new targets for novel therapeutic interventions of COPD. Such new treatments are urgently needed, because current COPD therapy does not alter the progressive deterioration of lung function. In this aspect, the perturbagen described herein is selected for targeting cell signaling pathways in COPD including hedgehog signaling, Notch signaling, the retinoic acid pathway, Wnt/β-catenin pathway, and the transforming growth factor-β (TGF-β pathway), etc.

In some embodiments, the present disclosure provides a method for treating chronic obstructive pulmonary disease and related conditions in a patient in need thereof. The method comprising a step of: (a) administering to a patient in need thereof a therapeutically effective amount of at least one perturbagen selected from Table 4, or a variant thereof; or (b) administering to a patient in need thereof a cell, the cell having been contacted with at least one perturbagen selected from Table 4, or a variant thereof.

In some embodiments, the present disclosure provides a method for treating COPD and related conditions in a patient in need thereof. This method includes (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. The at least one perturbagen selected from Table 4, or a variant thereof may be used for manufacture of a medicament for, COPD, and related conditions.

In some embodiments, the present disclosure provides a method for treating COPD and related conditions in a patient in need thereof. This method includes (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. In this aspect, altering the gene signature comprises a decrease in expression and/or activity in the progenitor cell of one or more genes designated as a “down” gene in the gene directionality column of Table 3. In one embodiment, the progenitor cell is a basal cell. In some embodiments, altering the gene signature comprises an activation of a network module designated in the network module column of Table 3. In other embodiments, the activation of the network module designated in the network module column of Table 3 comprises modulating expression and/or activity of 2 or more genes within a network module. In some embodiments, the activation of the network module designated in the network module column of Table 3 comprises modulating expression and/or activity of all of the genes within a network module. In other embodiments, the activation of the network module designated in the network module column of Table 3 comprises modulating expression and/or activity of 2 or more genes within 2 or more network modules.

In some embodiments, the present disclosure provides a method for treating chronic obstructive pulmonary disease and related conditions in a patient in need thereof. The method comprising a step of administering to a patient in need thereof a therapeutically effective amount of at least one perturbagen selected from Table 4. In some embodiments, the present disclosure provides a method for treating chronic obstructive pulmonary disease and related conditions in a patient in need thereof. The method comprising a step of administering to a patient in need thereof a cell, the cell having been contacted with at least one perturbagen selected from Table 4

In some embodiments, the present disclosure provides a method for treating COPD and related conditions in a patient in need thereof. This method includes (a) administering to a patient in need thereof a combination therapy having a conventional inhaled COPD therapy and at least one perturbagen selected from Table 4, or a variant thereof, wherein the combination therapy is capable of changing a gene signature in a progenitor cell, or (b) administering to a patient in need thereof a cell, the cell having been contacted with a combination therapy having a conventional inhaled COPD therapy and at least one perturbagen selected from Table 4, or a variant thereof, wherein the combination therapy is capable of changing a gene signature in a progenitor cell. In some embodiments, the combination therapy may be used for manufacture of a medicament for COPD and related conditions in a patient in need thereof. In this aspect, the conventional inhaled COPD therapy is selected from the group consisting of inhaled β2-agonist including levalbuterol, albuterol, formoterol, foradil, salmeterol, arformoterol; inhaled anticholinergics including ipratropium, triotropium; inhaled corticosteroids including beclomethasone, budesonide, fluticasone, triamcinolone; salmeterol and fluticasone; formoterol and budesonide; and theophylline.

In some embodiments, the present disclosure provides a method for treating COPD and related conditions in a patient in need thereof. This method includes (a) administering to a patient in need thereof a combination therapy having an antioxidant and at least one perturbagen selected from Table 4, or a variant thereof, wherein the at least one perturbagen is capable of changing a gene signature in a progenitor cell, or (b) administering to a patient in need thereof a cell, the cell having been contacted with a combination therapy having an antioxidant and at least one perturbagen selected from Table 4, or a variant thereof, wherein the combination therapy is capable of changing a gene signature in a progenitor cell. In some embodiments, the combination therapy may be used for manufacture of a medicament for, COPD, and related conditions. In this aspect, the antioxidant may be selected from the group consisting of flavonoids, glutathione, superoxide dismutase (SOD), n-acetyl-l-cysteine (NAC), phenolic antioxidant (hydroquinone and tert-butyl hydroquinone), quercetin, α-tocopherol, vitamin C, vitamin B6, vitamin D, vitamin E, and combinations thereof.

In some embodiments, the present disclosure provides a method for treating COPD and related conditions in a patient in need thereof. This method includes (a) administering to a patient in need thereof a combination therapy having at least one perturbagen selected from Table 4, or a variant thereof and a therapeutic agent targeting cell signaling pathways selected from the group consisting of hedgehog signaling inhibitor, Notch signaling agonist, the retinoic acid pathway inhibitor, TGF-β pathway inhibitor, and combinations thereof, wherein the at least one perturbagen is capable of changing a gene signature in a progenitor cell, or (b) administering to a patient in need thereof a cell, the cell having been contacted with a combination therapy having at least one perturbagen selected from Table 4, or a variant thereof and a therapeutic agent targeting cell signaling pathways selected from the group consisting of hedgehog signaling inhibitor, Notch signaling agonist, the retinoic acid pathway inhibitor, TGF-β pathway inhibitor, and combinations thereof, wherein the combination therapy is capable of changing a gene signature in a progenitor cell.

In some embodiments, for any herein described method, the COPD related conditions include emphysema, dyspnea, and chronic bronchitis.

In some embodiments, for any herein described methods, the present disclosure provides the use of the perturbagen of Table 4, or a variant thereof in the manufacture of a medicament in the form of aerosolized microparticles for treating COPD and related conditions in a patient in need thereof. In some embodiments, for any herein described methods, the present disclosure provides the use of the perturbagen of Table 4, or a variant thereof in the manufacture of a medicament in the form of aerosolized microparticles for treating emphysema in COPD patient. In some embodiments, for any herein described methods, the present disclosure provides the use of the perturbagen of Table 4, or a variant thereof in the manufacture of a medicament in the form of aerosolized microparticles for treating dyspnea in COPD patient. In some embodiments, for any herein described methods, the present disclosure provides the use of the perturbagen of Table 4, or a variant thereof in the manufacture of a medicament in the form of aerosolized microparticles for treating chronic bronchitis in COPD patient. In some embodiments, for any herein described methods, the present disclosure provides the use of the perturbagen of Table 4, or a variant thereof in the manufacture of a medicament in the form of aerosolized microparticles for clearing thick mucus in COPD patient.

In some embodiments, for any one of herein described methods, the aerosolized perturbagen microparticles are administered to the patient via the pulmonary airway using a device selected from the group consisting of nebulizer (e.g., jet nebulizer, ultrasound nebulizer, vibrating mesh nebulizer), metered-dose inhaler, and dry powder inhaler (DPI).

In some embodiments, for any one of herein described method, the administering occurs about once per day for one or more days. In embodiments, the administering occurs more than once per day for one or more days. In some embodiments, the administering occurs at most once per day for one or more days. In some embodiments, the administering occurs substantially continuously per administration period.

In some embodiments, for any herein described method, the present disclosure provides a pharmaceutical composition comprising at least one perturbagen that decreases the differentiation to goblet cells, increases the differentiation to ciliated cells without impacting other epithelial lineages.

In some embodiments, for any herein described method, the present disclosure provides a pharmaceutical composition comprising at least one perturbagen that restores differentiation of basal cells to mucus-clearing ciliated cells while simultaneously suppresses differentiation to mucus-secreting goblet cells thereby reduces the total mucus production, which furthermore ameliorates chronic infections and inflammation.

In some embodiments, for any herein described method, the treatment with at least one perturbagen based therapies provide one or more of: (a) decreased secretion of mucus by a goblet cell and (b) decreased synthesis of one or more mucins, optionally selected from MUC5AC, MUC5B, MUC2, MUC4, MUC7, MUC8, and MUC19.

### Asthma

Asthma is a chronic, or long-term condition that intermittently inflames and narrows the airways in the lungs. Asthma causes periods of wheezing, chest tightness, shortness of breath, and coughing. Asthma affects people of all ages and often starts during childhood.

Goblet cell hyperplasia (GCH) and/or goblet-cell metaplasia (GCM) have been established as a pathologic characteristic of mild, moderate, and severe asthma. The resulting hypersecretion of mucus associated with GCH/GCM causes airway narrowing and thus contributes to airflow obstruction.

The airway GCM is reported to be associated with the induction of pulmonary expression of Muc-5/5ac gene and mucin in murine models of allergic asthma (Allmam et al., Am. J. Respir. Cell Mol. Biol., 2000, vol. 22, pp. 253-260). Abnormalities in goblet cell number are accompanied by changes in stored and secreted mucin (MUC). The functional consequences of these changes in MUC stores and secretion can contribute to the pathophysiologic mechanisms for multiple clinical abnormalities in patients with asthma, including sputum production, airway narrowing, exacerbations, and accelerated loss in lung function. CD4(+) T cells and their T-helper type-2 cytokine products are important mediators of GCH, and MUC5AC is the dominant MUC gene that is expressed in goblet cells. The mechanism of cytokine-induced GCH, the relationships between MUC gene up-regulation and GCH, and the role of ion channels have been reported (Fahy, Chest, 2002, vol. 122, pp. 320S-326S).

COPD is also characterized by airflow obstruction and inflammation. However, the inflammatory process in asthma is markedly different from that in COPD.

Currently available asthma therapies include: short-acting β2-adrenoceptor agonists such as salbutamol; Long-acting beta-adrenoceptor agonists such as salmeterol and formoterol; inhaled anticholinergic medications such as ipratropium; inhaled adrenergic agonist such as epinephrine; corticosteroids, anti-leukotriene agents such as montelukast and zafirlukast; mast cell stabilizers such as cromolyn sodium; chloroquine; monoclonal antibody therapeutics, such as, Dupilumab and Benzaralizumab and combinations thereof.

In some embodiments, the present disclosure provides the use of perturbagen to probe the molecular mechanisms of GCH and goblet cell degranulation by assaying the genes expressions and or gene signature patterns induced by the perturbagen selected from Table 3. The gene signature discovered by the present disclosure provides new targets for novel therapeutic interventions of asthma. Such new treatments are urgently needed, because mucus hypersecretion is an important cause of morbidity and mortality in patients with asthma, and no specific treatments are available. In this aspect, the at least one perturbagen is selected for targeting cell signaling pathways in asthma including hedgehog signaling, Notch signaling, the retinoic acid pathway, Wnt/β-catenin pathway, TGF-β pathway, bone morphogenetic proteins (BMPs), growth differentiation factors and activins.

In some embodiments, the present disclosure provides a method for treating treatment of asthma in a patient in need thereof. This method includes (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. The at least one perturbagen selected from Table 4, or a variant thereof may be used for manufacture of a medicament for asthma and related conditions.

In some embodiments, the present disclosure provides a method for treating asthma and related conditions in a patient in need thereof. This method includes (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. In this aspect, altering the gene signature comprises a decrease in expression and/or activity in the progenitor cell of one or more genes designated as a “down” gene in the gene directionality column of Table 3. In one embodiment, the progenitor cell is a basal cell. In some embodiments, altering the gene signature comprises an activation of a network module designated in the network module column of Table 3. In other embodiments, the activation of the network module designated in the network module column of Table 3 comprises modulating expression and/or activity of 2 or more genes within a network module. In some embodiments, the activation of the network module designated in the network module column of Table 3 comprises modulating expression and/or activity of all of the genes within a network module. In other embodiments, the activation of the network module designated in the network module column of Table 3 comprises modulating expression and/or activity of 2 or more genes within 2 or more network modules.

In some embodiments, the present disclosure provides a method for treating treating asthma and related conditions in a patient in need thereof. The method comprising a step of administering to a patient in need thereof a therapeutically effective amount of at least one perturbagen selected from Table 4.

In some embodiments, the present disclosure provides a method for treating asthma and related conditions in a patient in need thereof. This method includes (a) administering to a patient in need thereof a combination therapy having a conventional inhaled asthma therapy and at least one perturbagen selected from Table 4, or a variant thereof, wherein the combination therapy is capable of changing a gene signature in a progenitor cell, or (b) administering to a patient in need thereof a cell, the cell having been contacted with a combination therapy having a conventional inhaled asthma management medicament and at least one perturbagen selected from Table 4, or a variant thereof, wherein the combination therapy is capable of changing a gene signature in a progenitor cell. The at least one perturbagen selected from Table 4, or a variant thereof may be used for manufacture of a medicament for asthma and related conditions. In this aspect, the conventional inhaled asthma therapy comprises a drug selected from the group consisting of inhaled 2-agonist including albuterol, formoterol; inhaled anticholinergics including ipratropium; inhaled corticosteroids, inhaled adrenergic agonist including epinephrine; corticosteroids, anti-leukotriene agents including montelukast and zafirlukast; mast cell stabilizers including cromolyn sodium; chloroquine; and combinations thereof.

In some embodiments, for any one of herein described method, the present disclosure provides the use of the perturbagen of Table 4, or a variant thereof in the manufacture of a medicament in the form of aerosolized microparticles for treating asthma. In some embodiments, for any one of herein described method, the present disclosure provides the use of the perturbagen of Table 4, or a variant thereof in the manufacture of a medicament in the form of aerosolized microparticles to control symptoms or prevent exacerbations in asthma patient.

In some embodiments, for any one of herein described method, the aerosolized perturbagen microparticles are administered to the patient via the pulmonary airway using a device selected from the group consisting of nebulizer (e.g., jet nebulizer, ultrasound nebulizer, vibrating mesh nebulizer), metered-dose inhaler, and dry powder inhaler (DPI).

In some embodiments, the present disclosure provides a method for treating asthma and related conditions in a patient in need thereof. The method comprising a step of: (a) administering to a patient in need thereof a therapeutically effective amount of at least one perturbagen selected from Table 4, or a variant thereof; or (b) administering to a patient in need thereof a cell, the cell having been contacted with at least one perturbagen selected from Table 4, or a variant thereof.

In some embodiments, for any one of herein described method, the administering occurs about once per day for one or more days. In embodiments, the administering occurs more than once per day for one or more days. In some embodiments, the administering occurs at most once per day for one or more days. In some embodiments, the administering occurs substantially continuously per administration period.

In some embodiments, for any herein described method, the present disclosure provides a pharmaceutical composition comprising at least one perturbagen that decreases the differentiation to goblet cells, increases the differentiation to ciliated cells without impacting other epithelial lineages.

In some embodiments, for any herein described method, the present disclosure provides a pharmaceutical composition comprising at least one perturbagen that restores differentiation of basal cells to mucus-clearing ciliated cells while simultaneously suppresses differentiation to mucus-secreting goblet cells thereby reduces the total mucus production, which furthermore ameliorates chronic infections and inflammation.

In some embodiments, for any herein described method, the treatment with at least one perturbagen based therapies provide one or more of: (a) decreased secretion of mucus by a goblet cell and (b) decreased synthesis of one or more mucins, optionally selected from MUC5AC, MUC5B, MUC2, MUC4, MUC7, MUC8, and MUC19.

### Severe, Glucocorticoid-Resistant Asthma

Corticosteroid (CS)-resistant or refractory (CSR) asthma was defined as less than 15% improvement in baseline forced expiratory volume in 1 second (FEV1) after a 14-day course of oral prednisolone (40 mg/d) in patients who demonstrate more than 15% improvement in FEV1 following the inhaled β2-agonist, salbutamol. Glucocorticoid insensitivity presents a profound management problem in patients with asthma because conventional therapies are not effective.

The thickness of the airway epithelium and basement membrane in patients with CSR asthma is greater than in corticosteroid sensitive (CSS) asthma, with similar levels of epithelial shedding. This difference was associated with altered expression of markers of epithelial proliferation, such as increased expression of Ki67, reduced retinoblastoma expression, and reduced expression of Bcl-2 (a negative regulator of epithelial cell death).

There are multiple molecular mechanisms underlying CSR asthma that may differ between patients, for example, reduced glucocorticoid receptor (GR) expression, GR affinity abnormality, loss of GR function due to abnormal GR phosphorylation induced by p38 mitogen-activated protein kinase (MAKP) activation, regulating GR responsiveness following IL-2 stimulation by Janus kinase 3/signal transducer and activator of transcription 5 (JAK3/STAT5), excessive activation of inflammatory transcription factors (e.g., AP-1), increased expression of c-Fos, c-Jun N-terminal kinase (JNK) activation, Cofilin-1 overexpression, activities of T-helper 2 (Th2) cytokines, reduced expression of transcriptional repressor cofactor including HDAC2 and Brahma-related gene Brg1. Genetic studies implicate the NF-κB pathway in CSR asthma. Certain cytokines, specifically IL-2, IL-4 and IL-13 that are overexpressed in patients with steroid resistant asthma, may induce a reduction in GR affinity in T lymphocytes, resulting in local resistance to the anti-inflammatory actions of corticosteroids. Five asthma genes or gene complexes have been identified such as ADAM33, PHF11, DPP10, GRRA, and SPINK5. The expression of ADAM33 is increased in the epithelium, smooth muscle, and submucosa of patients with CSR asthma and has been implicated in affecting airway remodeling in these patients. Polymorphisms in IL-4 signaling have also been associated with asthma severity and glucocorticoid responsiveness. The E375A and Q551R alleles in IL-4Rα are associated with severe asthma exacerbations and reduced lung function (Adcock et al., Current Allergy and Asthma Reports, 2008, vol. 8, pp. 171-178).

In addition, several related conditions are associated with recurrent exacerbations of treatment-insensitive asthma such as nasal sinus disease, gastroesophageal reflux, recurrent infection, psychological dysfunction, and obstructive sleep apnea. Treatment of the risk factors may affect subject's morbidity and mortality. Managing patients with CSR asthma poses considerable challenges. These patients are often subjected to the unwanted side effects of prolonged systemic glucocorticoid therapy without evidence of appreciable benefit. Further, the multiple mechanisms underlying CSR asthma may indicate a need for patient-specific treatment with novel therapies directed at abnormal signaling pathways to restore asthma control.

In some embodiments, the present disclosure provides the use of perturbagen to probe the molecular mechanisms of corticosteroid-resistance mechanism by assaying the genes expressions and or gene signature patterns induced by the perturbagen selected from Table 4. The gene signature discovered by the present disclosure provides new targets for novel therapeutic interventions of corticosteroid-resistant asthma. Such new treatments are urgently needed, because insensitivity to glucocorticoid treatment is an important cause of morbidity and mortality in patients with corticosteroid-resistant asthma, and conventional therapies are not effective. In this aspect, the molecular targets of the selected perturbagen for treating corticosteroid-resistant asthma include: at least one mechanism of action (MOA) selected from Table 4, restoring the ability of T-regulatory cells (Treg) cells from CSR subjects to release IL-10 at levels similar to those of CSS patients; antioxidants and/or NO synthase 2 inhibitor, NF-κB pathway inhibitor, NF-κB kinase 2 (IKK2) inhibitor, CXC chemokine receptor antagonist, PED4 inhibitor, JAK 3 kinase inhibitor, anti-IL-1 therapy, LXA4 therapy, anti-inflammatory cytokine therapy, and combinations thereof.

In some embodiments, the present disclosure provides a method for treating severe corticosteroid-resistant asthma and related conditions in a patient in need thereof. The method comprising a step of: (a) administering to a patient in need thereof a therapeutically effective amount of at least one perturbagen selected from Table 4, or a variant thereof; or (b) administering to a patient in need thereof a cell, the cell having been contacted with at least one perturbagen selected from Table 4, or a variant thereof.

In some embodiments, the present disclosure provides a method for treating severe corticosteroid-resistant asthma and related conditions in a patient in need thereof. This method includes the steps of: (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. The at least one perturbagen selected from Table 4, or a variant thereof may be used for manufacture of a medicament for, corticosteroid-resistant asthma, and related conditions.

In some embodiments, the present disclosure provides a method for treating severe corticosteroid-resistant asthma and related conditions in a patient in need thereof. This method includes the steps of: (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. In this aspect, altering the gene signature comprises a decrease in expression and/or activity in the progenitor cell of one or more genes designated as a “down” gene in the gene directionality column of Table 3. In one embodiment, the progenitor cell is a basal cell. In some embodiments, altering the gene signature comprises an activation of a network module designated in the network module column of Table 3. In other embodiments, the activation of the network module designated in the network module column of Table 3 comprises modulating expression and/or activity of 2 or more genes within a network module. In some embodiments, the activation of the network module designated in the network module column of Table 3 comprises modulating expression and/or activity of all of the genes within a network module. In other embodiments, the activation of the network module designated in the network module column of Table 3 comprises modulating expression and/or activity of 2 or more genes within 2 or more network modules.

In some embodiments, the present disclosure is a method for treating severe glucocorticoid-resistant asthma and related conditions in a patient in need thereof. This method includes a step of administering to a patient in need thereof a therapeutically effective amount of at least one perturbagen selected from Table 4. In some embodiments, the present disclosure is a method for treating severe, glucocorticoid-resistant asthma and related conditions in a patient in need thereof. The method comprising a step of administering to a patient in need thereof a cell, the cell having been contacted with at least one perturbagen selected from Table 4

In some embodiments, the present disclosure provides a method for treating severe corticosteroid-resistant asthma and related conditions in a patient in need thereof. This method includes (a) administering to a patient in need thereof a combination therapy having a conventional inhaled asthma therapy and at least one perturbagen selected from Table 4, or a variant thereof, wherein the combination therapy is capable of changing a gene signature in a progenitor cell, or (b) administering to a patient in need thereof a cell, the cell having been contacted with a combination therapy having a conventional inhaled asthma therapy and at least one perturbagen selected from Table 4, or a variant thereof, wherein the combination therapy is capable of changing a gene signature in a progenitor cell. The combination therapy may be used for manufacture of a medicament for, corticosteroid-resistant, and related conditions. In this aspect, the conventional inhaled asthma therapy comprises a drug selected from the group consisting of inhaled β2-agonist including albuterol, formoterol; inhaled anticholinergics including ipratropium; inhaled corticosteroids, inhaled adrenergic agonist including epinephrine; corticosteroids, anti-leukotriene agents including montelukast and zafirlukast; mast cell stabilizers including cromolyn sodium; chloroquine; and combinations thereof.

In some embodiments, the present disclosure provides a method for treating severe corticosteroid-resistant asthma and related conditions in a patient in need thereof. This method includes the steps of (a) administering to a patient in need thereof a combination therapy having a targeted therapy for CSR and at least one perturbagen selected from Table 4, or a variant thereof, wherein the combination therapy is capable of changing a gene signature in a progenitor cell or (b) administering to a patient in need thereof a cell, the cell having been contacted with a combination therapy having a targeted therapy for CSR and at least one perturbagen selected from Table 4, or a variant thereof, wherein the combination therapy is capable of changing a gene signature in a progenitor cell. The combination therapy may be used for manufacture of a medicament for, corticosteroid-resistant asthma, and related conditions. In this aspect, the targeted therapy for CSR may include combination of vitamin D3 with dexamethasone; antioxidants, NO synthase 2 inhibitor, NF-κB pathway inhibitor, NF-κB kinase 2 (IKK2, IKKβ) inhibitor, CXC chemokine receptor antagonist, PED4 inhibitor, JAK 3 kinase inhibitor, anti-IL-1 therapy, LXA4 therapy, anti-inflammatory cytokine therapy, TNF-α receptor blocker, and combinations thereof.

In some embodiments, the present disclosure provides a method for treating severe corticosteroid-resistant asthma and related conditions in a patient in need thereof. This method includes the steps of: (a) administering to a patient in need thereof a combination therapy having a NF-κB pathway inhibitor and/or a NF-κB kinase 2 (IKK2) inhibitor and at least one perturbagen selected from Table 4, or a variant thereof, wherein the combination therapy is capable of changing a gene signature in a progenitor cell, or (b) administering to a patient in need thereof a cell, the cell having been contacted with a combination therapy having a NF-κB pathway inhibitor and/or a NF-κB kinase 2 (IKK2) inhibitor and at least one perturbagen selected from Table 4, or a variant thereof, wherein the combination therapy is capable of changing a gene signature in a progenitor cell. The combination therapy may be used for manufacture of a medicament for, corticosteroid-resistant asthma, and related conditions. In this aspect, the NF-κB pathway inhibitor and/or NF-κB kinase 2 (IKK2) inhibitor is selected from the group consisting of BI605906, MLN120B, PHA-408, TPCA-1, SC-514, LY2409881, PS-1145, IMD-0354, ACHP, BMS-345541, withaferin A, BOT-64, ainsliadimer A, and combinations thereof.

In some embodiments, the present disclosure provides a method for treating severe corticosteroid-resistant asthma in a patient in need thereof. This method includes the stpes of: (a) administering to a patient in need thereof a combination therapy having a proteasome inhibitor and at least one perturbagen selected from Table 4, or a variant thereof, wherein the combination therapy is capable of changing a gene signature in a progenitor cell or (b) administering to a patient in need thereof a cell, the cell having been contacted with a combination therapy having a proteasome inhibitor and at least one perturbagen selected from Table 4, or a variant thereof, wherein the combination therapy is capable of changing a gene signature in a progenitor cell. The combination therapy may be used for manufacture of a medicament for, corticosteroid-resistant asthma, and related conditions. In this aspect, the proteasome inhibitor may include cyclosporine A, ubiquitin ligase inhibitors, boronic acid peptide, bortezomib, FK506 (tacrolimus), disulfiram, and combinations thereof.

In some embodiments, the present disclosure provides a method for treating severe corticosteroid-resistant asthma in a patient in need thereof. This method includes the steps of: (a) administering to a patient in need thereof a combination therapy having an antioxidant and at least one perturbagen selected from Table 4, or a variant thereof, wherein the at least one perturbagen is capable of changing a gene signature in a progenitor cell, or (b) administering to a patient in need thereof a cell, the cell having been contacted with a combination therapy having an antioxidant and at least one perturbagen selected from Table 4, or a variant thereof, wherein the combination therapy is capable of changing a gene signature in a progenitor cell. The combination therapy may be used for manufacture of a medicament for, corticosteroid-resistant asthma, and related conditions. In this aspect, the antioxidant may be selected from the group consisting of flavonoids, glutathione, superoxide dismutase (SOD), n-acetyl-l-cysteine (NAC), phenolic antioxidant (hydroquinone and tert-butyl hydroquinone), quercetin, α-tocopherol, vitamin C, vitamin B6, vitamin D, vitamin E, and combinations thereof.

In some embodiments, the present disclosure provides the use of the perturbagen of Table 4, or a variant thereof in the manufacture of a medicament in the form of aerosolized microparticles for treating corticosteroid-resistant asthma.

In some embodiments, the present disclosure provides the use of the perturbagen of Table 4, or a variant thereof in the manufacture of a medicament in the form of aerosolized microparticles to control symptoms or related conditions in corticosteroid-resistant asthma patient. In this aspect, the related conditions in corticosteroid-resistant asthma patient include nasal sinus disease, gastroesophageal reflux, recurrent infection, psychological dysfunction, and obstructive sleep apnea. Treatment of the herein described CRS related conditions in corticosteroid-resistant asthma may reduce subject's morbidity and mortality.

In some embodiments, for any one of herein described methods, the aerosolized perturbagen microparticles are administered to the patient via the pulmonary airway using a device selected from the group consisting of nebulizer (e.g., jet nebulizer, ultrasound nebulizer, vibrating mesh nebulizer), metered-dose inhaler, and dry powder inhaler (DPI).

In some embodiments, the present disclosure provides a method for making a therapeutic agent for severe glucocorticoid-resistant asthma. The method includes the steps of: (a) identifying a therapeutic agent for therapy according to any of the embodiments disclosed herein, and (b) formulating the candidate perturbation as a therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into ciliated cells or immediate progenitors thereof following exposure of the population of cells to the perturbation; and identifying the perturbation as a candidate perturbation for promoting the transition of a population of progenitor cells into ciliated cells or immediate progenitors thereof based on the perturbation signature. Further, in this aspect, the perturbation signature is an increase in expression and/or activity in the progenitor cell of one or more genes selected from in an activation of a network module designated in the network module column of Table 3. In some embodiments, the activation of one or more genes of the network module designated in the network module column of Table 3 comprises modulating expression and/or activity of 2, 3, 4, 5, 6 7, 8, 9, 10 or more genes within a network module. In some embodiments, altering the gene signature comprises a decrease in expression and/or activity in the progenitor cell of one or more genes designated as a “down” gene in the gene directionality column of Table 3. In some embodiments, altering the gene signature comprises a decrease in expression and/or activity in the progenitor cell of two or more genes designated as a “down” gene in the gene directionality column of Table 3. When at least one of the perturbation signatures selected from Table 3 is detected by the herein described method, the therapeutic agent is selected for making the medicament to treat the patient with severe glucocorticoid-resistant asthma and related conditions in a patient in need thereof.

In some embodiments, for any one of herein described method, the administering occurs about once per day for one or more days. In embodiments, the administering occurs more than once per day for one or more days. In some embodiments, the administering occurs at most once per day for one or more days. In some embodiments, the administering occurs substantially continuously per administration period.

In some embodiments, for any herein described method, the present disclosure provides a pharmaceutical composition comprising at least one perturbagen that decreases the differentiation to goblet cells, increases the differentiation to ciliated cells without impacting other epithelial lineages.

In some embodiments, for any herein described method, the present disclosure provides a pharmaceutical composition comprising at least one perturbagen that restores differentiation of basal cells to mucus-clearing ciliated cells while simultaneously suppresses differentiation to mucus-secreting goblet cells thereby reduces the total mucus production, which furthermore ameliorates chronic infections and inflammation.

In some embodiments, for any herein described method, the treatment with at least one perturbagen based therapies provide one or more of: (a) decreased secretion of mucus by a goblet cell and (b) decreased synthesis of one or more mucins, optionally selected from MUC5AC, MUC5B, MUC2, MUC4, MUC7, MUC8, and MUC19.

### Cystic Fibrosis (CF)

Cystic fibrosis (CF) is an autosomal recessive genetic disease caused by mutations in the cystic fibrosis transmembrane regulator (CFTR) gene, which has important roles in ion exchange. Cystic fibrosis is an inherited disease characterized by the buildup of thick, sticky mucus that can clog the airways, leading to severe problems with breathing and bacterial infections in the lungs. These infections cause chronic coughing, wheezing, and inflammation. Over time, mucus buildup and infections result in permanent lung damage, including the formation of scar tissue (fibrosis) and cysts in the lungs.

The CFTR protein is located in the apical surface of airway, intestinal, and exocrine epithelial cells. In the lungs, functional CFTR protein ensures optimal volume, electrolyte composition, and pH of the airway surface liquid (ASL), a thin fluid layer protecting the epithelium from inspired air. Mutations in the CFTR gene (e.g., Arg553X, Gly542X, Trp1282X, Phe508del, Gly85Glu, Arg560Thr, Ile507del, Asn1303Lys, Gly551Asp, Arg117His and Arg347Pro) disrupt the function of the chloride channels, preventing them from regulating the flow of chloride ions and water across cell membranes. As a result, cells that line the passageways of the lungs, produce mucus that is unusually thick and sticky (Elborn et al., Advances in Genomics and Genetics, 2014, pp. 161-172). Even patients bearing the exactly the same CFTR genotype may differ in their clinical response to treatment, calling for personalized therapy.

The currently available therapeutic treatment for cystic fibrosis targeting symptom management rather than the fundamental cellular defect (See Elborn et al. supra). Mucolytic, hydrating, and bronchodilator agents are used to facilitate lung mucus clearance, antibiotics are prescribed to control chronic respiratory infections or to treat acute episodes of infective exacerbations, and anti-inflammatory drugs are used to reduce the deleterious inflammatory reaction. Pancreatic enzymes and vitamin supplements are prescribed for patients who are pancreatic insufficient in order to optimize their nutritional status. New mutation specific therapies have been approved for the patients bearing the most common F508del CFTR mutation in homozygosis include CFTR corrector Lumacaftor and the potentiator ivacaftor.

In some embodiments, the present disclosure provides the use of perturbagen to probe the molecular mechanisms of CFTR gene mutations by assaying the genes expressions and or gene signature patterns induced by the perturbagen.

The gene signature discovered by the present disclosure provides new targets for novel therapeutic interventions of cystic fibrosis. Such new treatments are urgently needed, because currently available therapeutic treatment for cystic fibrosis targeting symptom management rather than the fundamental cellular defect.

In some embodiments, the present disclosure provides a method for treating cystic fibrosis and related conditions in a patient in need thereof. This method includes the steps of: (a) administering to a patient in need thereof a therapeutically effective amount of at least one perturbagen selected from Table 4, or a variant thereof; or (b) administering to a patient in need thereof a cell, the cell having been contacted with at least one perturbagen selected from Table 4, or a variant thereof.

In some embodiments, the present disclosure provides a method for treating cystic fibrosis and related conditions in a patient in need thereof. This method includes (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. The at least one perturbagen selected from Table 4, or a variant thereof may be used for manufacture of a medicament for, cystic fibrosis, and related conditions.

In some embodiments, the present disclosure provides for methods of treatment of cystic fibrosis and related conditions in a patient in need thereof. This method includes the steps of: (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. In this aspect, altering the gene signature comprises a decrease in expression and/or activity in the progenitor cell of one or more genes designated as a “down” gene in the gene directionality column of Table 3. In one embodiment, the progenitor cell is a basal cell. In some embodiments, altering the gene signature comprises an activation of a network module designated in the network module column of Table 3. In other embodiments, the activation of the network module designated in the network module column of Table 3 comprises modulating expression and/or activity of 2 or more genes within a network module. In some embodiments, the activation of the network module designated in the network module column of Table 3 comprises modulating expression and/or activity of all of the genes within a network module. In other embodiments, the activation of the network module designated in the network module column of Table 3 comprises modulating expression and/or activity of 2 or more genes within 2 or more network modules.

In some embodiments, the present disclosure is a method for treating cystic fibrosis and related conditions in a patient in need thereof. This method includes a step of administering to a patient in need thereof a therapeutically effective amount of at least one perturbagen selected from Table 4. The method comprising a step of administering to a patient in need thereof a cell, the cell having been contacted with at least one perturbagen selected from Table 4.

In some embodiments, the present disclosure provides a method for treating cystic fibrosis and related conditions in a patient in need thereof. This method includes the steps of (a) administering to a patient in need thereof a combination therapy having a conventional cystic-fibrosis therapy and at least one perturbagen selected from Table 4, or a variant thereof, wherein the combination therapy is capable of changing a gene signature in a progenitor cell, or (b) administering to a patient in need thereof a cell, the cell having been contacted with a combination therapy having a conventional cystic-fibrosis therapy and at least one perturbagen selected from Table 4, or a variant thereof, wherein the combination therapy is capable of changing a gene signature in a progenitor cell. The combination therapy may be used for manufacture of a medicament for, corticosteroid-resistant, and related conditions. In this aspect, the conventional cystic-fibrosis therapy comprises a drug selected from the group consisting of bronchodilator agents, antibiotics, ivacaftor, lumacaftor, anti-inflammatory drugs, and combinations thereof.

In some embodiments, the present disclosure provides the use of the perturbagen of Table 4, or a variant thereof in the manufacture of a medicament in the form of aerosolized microparticles for treating cystic fibrosis. In some embodiments, the present disclosure provides the use of the perturbagen of Table 4, or a variant thereof in the manufacture of a medicament in the form of aerosolized microparticles to control symptoms in patient having cystic fibrosis. In some embodiments, for any one of herein described methods, the aerosolized perturbagen microparticles are administered to the patient via the pulmonary airway using a device selected from the group consisting of nebulizer (e.g., jet nebulizer, ultrasound nebulizer, vibrating mesh nebulizer), metered-dose inhaler, and dry powder inhaler (DPI).

An aspect of the present disclosure is related to a method for selecting the patient for any herein described methods.

This method includes the steps of obtaining from a subject having cystic fibrosis a sample of cells comprising a basal cell; and contacting the sample of cells with least one perturbagen selected from Table 4, or a variant thereof. In this aspect, when the at least one perturbagen alters a gene signature in the sample of cells, the subject is selected as a patient.

In some embodiments, the present disclosure provides a method for selecting the patient for any herein described methods. This method includes the steps of obtaining from a subject having cystic fibrosis a sample of cells comprising a basal cell; and contacting the sample of cells with at least one perturbagen selected from Table 4, or a variant thereof.

In this aspect, when the at least one perturbagen decreases in the sample of cells the expression and/or activity of one or more genes selected from Table 3 designated as a “down” gene in the gene directionality column of Table 3, the subject is selected as a patient. In this aspect, altering the gene signature comprises an activation of a network module designated in the network module column of Table 3. In other embodiments, the activation of the network module designated in the network module column of Table 3 comprises modulating expression and/or activity of 2 or more genes within a network module. In some embodiments, the activation of the network module designated in the network module column of Table 3 comprises modulating expression and/or activity of all of the genes within a network module.

In other embodiments, the activation of the network module designated in the network module column of Table 3 comprises modulating expression and/or activity of 2 or more genes within 2 or more network modules.

In some embodiments, the present disclosure provides a patient-specific therapy for cystic fibirosis and related conditions in a patient in need thereof. In this aspect, the present disclosure provides a method for selecting the patient having a CFTR mutation genotype for any herein described methods. This method includes the steps of obtaining from a subject having cystic fibrosis a biological sample and isolating DNA from the biological sample, and performing genotyping assay by amplifying the isolated DNA, detecting the presence of a mutation of CFTR gene in the amplified DNA. In this aspect, when the patient has one or more the single site of mutation in CFTR gene DNA sequence selected from the group consisting of Arg553X, Gly542X, Trp1282X, Phe508del, Gly85Glu, Arg560Thr, Ile507del, Asn1303Lys, Gly551Asp, Arg117His, and Arg347Pro, the subject is selected as a patient. In some embodiments, the biological sample is a blood sample or airway epithelium tissue in the lung.

Genotyping assays for detecting mutation in amplified DNA sequence are well known in the art. Non-limiting examples include peptide mass signature genotyping (PMSG), real time PCR such as Applied Biosystems™ TaqMan® Genotyping Assays may be used for the detection of DNA mutations in the cystic fibrosis transmembrane conductance regulator (CFTR) gene.

In some embodiments, the present disclosure provides a method for determining efficacy of the herein described perturbagen in the patient with a CFTR mutation genotype selected from the group consisting Arg553X, Gly542X, Trp1282X, Phe508del, Gly85Glu, Arg560Thr, Ile507del, Asn1303Lys, Gly551Asp, Arg117His, and Arg347Pro. In this aspect, the method comprises the steps of: obtaining from the CF patient with the genotype of CFTR mutation as described herein a sample of cells comprising a basal cell; and contacting the sample of cells with the herein described therapeutic agent; identifying a perturbation signature for the herein described therapeutic agent, the perturbation signature comprising one or more cellular-components and a significance score associated with each cellular-component, the significance score of each cellular-component quantifying an association between a change in expression of the cellular-component and a change in cell fate of the population of the population of progenitor cells into ciliated cells or immediate progenitors thereof following exposure of the population of cells to the perturbation; and identifying the perturbation as a candidate perturbation for promoting the transition of a population of progenitor cells into ciliated cells or immediate progenitors thereof based on the perturbation signature. Further, in this aspect, the perturbation signature is an increase in expression and/or activity in the progenitor cell of one or more genes selected from in an activation of a network module designated in the network module column of Table 3. In some embodiments, the activation of one or more genes of the network module designated in the network module column of Table 3 comprises modulating expression and/or activity of 2, 3, 4, 5, 6 7, 8, 9, 10 or more genes within a network module. In some embodiments, altering the gene signature comprises a decrease in expression and/or activity in the progenitor cell of one or more genes designated as a “down” gene in the gene directionality column of Table 3. In some embodiments, altering the gene signature comprises a decrease in expression and/or activity in the progenitor cell of two or more genes designated as a “down” gene in the gene directionality column of Table 3. In this aspect, the detection of the perturbation signature indicates therapeutic efficacy in the patient with the CFTR mutation genotype as herein described, and a therapeutically effective amount of the therapeutic agent is administered to this patient.

In some embodiments, the present disclosure provides a method for making a therapeutic agent for cystic fibrosis in a patient having a CFTR mutation genotype selected from the group consisting of Arg553X, Gly542X, Trp1282X, Phe508del, Gly85Glu, Arg560Thr, Ile507del, Asn1303Lys, Gly551Asp, Arg117His, and Arg347Pro. The method comprises (a) identifying a therapeutic agent for therapy according to any of the embodiments disclosed herein, and (b) formulating the candidate perturbation as a therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into ciliated cells or immediate progenitors thereof following exposure of the population of cells to the perturbation; and identifying the perturbation as a candidate perturbation for promoting the transition of a population of progenitor cells into ciliated cells or immediate progenitors thereof based on the perturbation signature. Further, in this aspect, the perturbation signature is an increase in expression and/or activity in the progenitor cell of one or more genes selected from in an activation of a network module designated in the network module column of Table 3. In some embodiments, the activation of one or more genes of the network module designated in the network module column of Table 3 comprises modulating expression and/or activity of 2, 3, 4, 5, 6 7, 8, 9, 10 or more genes within a network module. In some embodiments, altering the gene signature comprises a decrease in expression and/or activity in the progenitor cell of one or more genes designated as a “down” gene in the gene directionality column of Table 3. In some embodiments, altering the gene signature comprises a decrease in expression and/or activity in the progenitor cell of two or more genes designated as a “down” gene in the gene directionality column of Table 3. When at least one of the perturbation signatures selected from Tables 1 is detected by the herein described method, the therapeutic agent is selected for making the medicament to treat the patient with severe glucocorticoid-resistant asthma.

In some embodiments, for any one of herein described method, the administering occurs about once per day for one or more days. In embodiments, the administering occurs more than once per day for one or more days. In some embodiments, the administering occurs at most once per day for one or more days. In some embodiments, the administering occurs substantially continuously per administration period.

In some embodiments, for any herein described method, the present disclosure provides a pharmaceutical composition comprising at least one perturbagen that decreases the differentiation to goblet cells, increases the differentiation to ciliated cells without impacting other epithelial lineages.

In some embodiments, for any herein described method, the present disclosure provides a pharmaceutical composition comprising at least one perturbagen that restores differentiation of basal cells to mucus-clearing ciliated cells while simultaneously suppresses differentiation to mucus-secreting goblet cells thereby reduces the total mucus production, which furthermore ameliorates chronic infections and inflammation.

In some embodiments, for any herein described method, the treatment with at least one perturbagen based therapies provide one or more of: (a) decreased secretion of mucus by a goblet cell and (b) decreased synthesis of one or more mucins, optionally selected from MUC5AC, MUC5B, MUC2, MUC4, MUC7, MUC8, and MUC19.

### Primary Ciliary Dyskinesia

Primary ciliary dyskinesia (PCD) (also known as Immotile Cilia Syndrome) is an inherited disorder of motile cilia and sperm flagella that causes impaired clearance of mucus and debris.

PCD is characterized by recurrent respiratory infections such as bronchitis and/or pneumonias, chronic cough, chronic wheezing, excess mucus, chronic nasal congestion, difficulty clearing mucus. The current therapeutic treatment for PCD includes saline nasal washes, anti-inflammatory nasal sprays and nasal/sinus surgery for sinus infection, or bronchodilators, mucolytics, antibiotics and steroids for preventing or delaying progressive and/or advanced lung diseases. There is no cure for PCD and the current treatment slows the progression of the disease and remove trapped mucus from the lungs and airways.

PCD and CF are both autosomal recessive genetic diseases. However, there are a number of differences in both pathogenesis and etiology. CF is associated with defects of a single gene, the CFTR gene, whereas PCD is a genetically heterogeneous condition with mutations in 27 known genes including DNAH1, DNAH5, DNA/I1, ZMYND10, DYX1C1, CCDC39, CCDC40, ARMC4 which cause defects of the dynein proteins.

In some embodiments, the present disclosure provides the use of perturbagen to probe the molecular mechanisms of PCD by assaying the genes expressions and or gene signature patterns induced by the perturbagen selected from Table 3. The gene signature discovered by the present disclosure provides new targets for novel therapeutic interventions of primary ciliary dyskinesia. Such new treatments are urgently needed, because there is no cure for PCD and the current treatment only slows the progression of the disease and remove trapped mucus from the lungs and airways.

In some embodiments, the present disclosure is a method for treating primary ciliary dyskinesia and related conditions in a patient in need thereof. The method comprising a step of: (a) administering to a patient in need thereof a therapeutically effective amount of at least one perturbagen selected from Table 4, or a variant thereof; or (b) administering to a patient in need thereof a cell, the cell having been contacted with at least one perturbagen selected from Table 4, or a variant thereof.

In some embodiments, the present disclosure provides for methods of treatment of PCD and related conditions in a patient in need thereof. This method includes (a) administering to a patient in need thereof at least one perturbagen selected from Table 4, or a variant thereof, wherein the at least one perturbagen is capable of changing a gene signature in a progenitor cell or (b) administering to a patient in need thereof a cell, the cell having been contacted with at least one perturbagen selected from Table 4, or a variant thereof, wherein the at least one perturbagen is capable of changing a gene signature in a progenitor cell. The at least one perturbagen selected from Table 4, or a variant thereof may be used for manufacture of a medicament for, PCD, and related conditions.

In some embodiments, the present disclosure provides for methods of treatment of PCD and related conditions in a patient in need thereof. This method includes (a) administering to a patient in need thereof at least one perturbagen selected from Table 4, or a variant thereof, wherein the at least one perturbagen is capable of changing a gene signature in a progenitor cell or (b) administering to a patient in need thereof a cell, the cell having been contacted with at least one perturbagen selected from Table 4, or a variant thereof, wherein the at least one perturbagen is capable of changing a gene signature in a progenitor cell. In this aspect, altering the gene signature comprises a decrease in expression and/or activity in the progenitor cell of one or more genes designated as a “down” gene in the gene directionality column of Table 3. In one embodiment, the progenitor cell is a basal cell. In some embodiments, altering the gene signature comprises an activation of a network module designated in the network module column of Table 3.

In other embodiments, the activation of the network module designated in the network module column of Table 3 comprises modulating expression and/or activity of 2 or more genes within a network module. In some embodiments, the activation of the network module designated in the network module column of Table 3 comprises modulating expression and/or activity of all of the genes within a network module. In other embodiments, the activation of the network module designated in the network module column of Table 3 comprises modulating expression and/or activity of 2 or more genes within 2 or more network modules.

In some embodiments, the present disclosure is a method for treating primary ciliary dyskinesia and related conditions in a patient in need thereof. The method comprising a step of administering to a patient in need thereof a therapeutically effective amount of at least one perturbagen selected from Table 4. In some embodiments, the present disclosure is a method for treating primary ciliary dyskinesia and related conditions in a patient in need thereof. The method comprising a step of administering to a patient in need thereof a cell, the cell having been contacted with at least one perturbagen selected from Table 4.

In some embodiments, the present disclosure provides a method for treating PCD and related conditions in a patient in need thereof. This method includes the steps of (a) administering to a patient in need thereof a combination therapy having a conventional PCD therapy and at least one perturbagen selected from Table 4, or a variant thereof, wherein the combination therapy is capable of changing a gene signature in a progenitor cell, or (b) administering to a patient in need thereof a cell, the cell having been contacted with a combination therapy having a conventional PCD therapy and at least one perturbagen selected from Table 4, or a variant thereof, wherein the combination therapy is capable of changing a gene signature in a progenitor cell. The combination therapy may be used for manufacture of a medicament for, PCD and related conditions. In this aspect, the conventional PCD therapy comprises a drug selected from the group consisting of saline nasal washes, anti-inflammatory nasal sprays, nasal/sinus surgery for sinus infection, bronchodilators, mucolytics, antibiotics, steroids, and combinations thereof.

In some embodiments, the present disclosure provides the use of the perturbagen of Table 4, or a variant thereof in the manufacture of a medicament in the form of aerosolized microparticles for treating PCD. In some embodiments, the present disclosure provides the use of the perturbagen of Table 4, or a variant thereof in the manufacture of a medicament in the form of aerosolized microparticles to control symptoms in PCD patient. In some embodiments, for any one of herein described methods, the aerosolized perturbagen microparticles are administered to the patient via the pulmonary airway using a device selected from the group consisting of nebulizer (e.g., jet nebulizer, ultrasound nebulizer, vibrating mesh nebulizer), metered-dose inhaler, and dry powder inhaler (DPI).

An aspect of the present disclosure is related to a method for selecting the patient for any herein described methods.

This method includes the steps of obtaining from a subject having PCD a sample of cells comprising a basal cell; and contacting the sample of cells with least one perturbagen selected from Table 4, or a variant thereof. In this aspect, when the at least one perturbagen alters a gene signature in the sample of cells, the subject is selected as a patient.

In some embodiments, the present disclosure provides a method for selecting the patient having PCD for any herein described methods. This method includes the steps of obtaining from a subject having PCD a sample of cells comprising a basal cell; and contacting the sample of cells with at least one perturbagen selected from Table 4, or a variant thereof. In this aspect, when the at least one perturbagen decreases in the sample of cells the expression and/or activity of one or more genes selected from Table 3 designated as a “down” gene in the gene directionality column of Table 3, the subject is selected as a patient.

In some embodiments, for any one of herein described method, the administering occurs about once per day for one or more days. In embodiments, the administering occurs more than once per day for one or more days. In some embodiments, the administering occurs at most once per day for one or more days. In some embodiments, the administering occurs substantially continuously per administration period.

In some embodiments, for any herein described method, the present disclosure provides a pharmaceutical composition comprising at least one perturbagen that decreases the differentiation to goblet cells, increases the differentiation to ciliated cells without impacting other epithelial lineages.

In some embodiments, for any herein described method, the present disclosure provides a pharmaceutical composition comprising at least one perturbagen that restores differentiation of basal cells to mucus-clearing ciliated cells while simultaneously suppresses differentiation to mucus-secreting goblet cells thereby reduces the total mucus production, which furthermore ameliorates chronic infections and inflammation.

In some embodiments, for any herein described method, the treatment with at least one perturbagen based therapies provide one or more of: (a) decreased secretion of mucus by a goblet cell and (b) decreased synthesis of one or more mucins, optionally selected from MUC5AC, MUC5B, MUC2, MUC4, MUC7, MUC8, and MUC19.

### Non-Cystic Fibrosis Bronchiectasis

Non-cystic fibrosis bronchiectasis (NCFBE) is a chronic inflammatory lung disease characterized by irreversible dilation of the bronchi, symptoms of persistent cough and expectoration, and recurrent infective exacerbations. Pseudomonas aeruginosa infections are associated with the most severe forms of bronchiectasis.

NCFBE is a heterogeneous, chronic condition with many etiologies such as including idiopathic (up to 50% of cases), post-respiratory tract infection, rare immunodeficiency disorders, genetic abnormalities, autoimmune conditions, chronic inflammation, and mechanical obstruction. It poses a significant burden on patients and healthcare practitioners and services. Clinical exacerbations often result in reduced quality of life, increased rate of lung function decline, increased hospitalization, and mortality. The mainstay of bronchiectasis management is improving symptoms and reducing exacerbations. The current available NCFBE therapies include: antibiotic, saline, inhaled mannitol, mucolytics, carbocisteine, non-steroidal anti-inflammatory drugs (NSAIDs), leukotriene receptor antagonists, ibuprofen; long-acting β2 agonists and inhaled corticosteroids; macrolide, bronchodilator including β2 adrenoreceptor agonists.

In some embodiments, the present disclosure provides the use of perturbagen to probe the molecular mechanisms of NCFBE by assaying the genes expressions and or gene signature patterns induced by the perturbagen selected from Tables 1. The gene signature discovered by the present disclosure provides new targets for novel therapeutic interventions of non-cystic fibrosis bronchiectasis. Such new treatments are urgently needed because there is no cure NCFBE.

In some embodiments, the present disclosure is a method for treating non-cystic fibrosis bronchiectasis and related conditions in a patient in need thereof. This method includes the steps of: (a) administering to a patient in need thereof a therapeutically effective amount of at least one perturbagen selected from Table 4, or a variant thereof; or (b) administering to a patient in need thereof a cell, the cell having been contacted with at least one perturbagen selected from Table 4, or a variant thereof.

In some embodiments, the present disclosure provides a method for treating non-cystic fibrosis bronchiectasis and related conditions in a patient in need thereof. This method includes the steps of: (a) administering to a patient in need thereof at least one perturbagen selected from Table 4, or a variant thereof, wherein the at least one perturbagen is capable of changing a gene signature in a progenitor cell, or (b) administering to a patient in need thereof a cell, the cell having been contacted with at least one perturbagen selected from Table 4, or a variant thereof, wherein the at least one perturbagen is capable of changing a gene signature in a progenitor cell. The at least one perturbagen selected from Table 4, or a variant thereof may be used for manufacture of a medicament for, non-cystic fibrosis bronchiectasis, and related conditions.

In some embodiments, the present disclosure is a method for treating non-cystic fibrosis bronchiectasis and related conditions in a patient in need thereof. This method includes a step of administering to a patient in need thereof a therapeutically effective amount of at least one perturbagen selected from Table 4. In some embodiments, the present disclosure is a method for treating non-cystic fibrosis bronchiectasis and related conditions in a patient in need thereof.

The method comprising a step of administering to a patient in need thereof a cell, the cell having been contacted with at least one perturbagen selected from Table 4 In some embodiments, the present disclosure provides a method for treating non-cystic fibrosis bronchiectasis and related conditions in a patient in need thereof. This method includes the steps of (a) administering to a patient in need thereof a combination therapy having a conventional non-cystic fibrosis bronchiectasis therapy and at least one perturbagen selected from Table 4, or a variant thereof, wherein the combination therapy is capable of changing a gene signature in a progenitor cell, or (b) administering to a patient in need thereof a cell, the cell having been contacted with a combination therapy having a conventional non-cystic fibrosis bronchiectasis therapy and at least one perturbagen selected from Table 4, or a variant thereof, wherein the combination therapy is capable of changing a gene signature in a progenitor cell. The combination therapy may be used for manufacture of a medicament for, corticosteroid-resistant, and related conditions. In this aspect, the conventional non-cystic fibrosis bronchiectasis therapy comprises a drug selected from the group consisting of bronchodilator agents; antibiotics; anti-inflammatory drugs; ibuprofen; saline; inhaled mannitol; mucolytics; carbocisteine; NSAID; long-acting β2 agonists and inhaled corticosteroids; macrolide; bronchodilator including β2 adrenoreceptor agonist; and combinations thereof.

In some embodiments, the present disclosure provides the use of the perturbagen of Table 4, or a variant thereof in the manufacture of a medicament in the form of aerosolized microparticles for treating non-cystic fibrosis bronchiectasis. In some embodiments, the present disclosure provides the use of the perturbagen of Table 4, or a variant thereof in the manufacture of a medicament in the form of aerosolized microparticles to control symptoms or related conditions in non-cystic fibrosis bronchiectasis patient.

In some embodiments, for any one of herein described methods, the aerosolized perturbagen microparticles are administered to the patient via the pulmonary airway using a device selected from the group consisting of nebulizer (e.g., jet nebulizer, ultrasound nebulizer, vibrating mesh nebulizer), metered-dose inhaler, and dry powder inhaler (DPI).

In some embodiments, for any one of herein described method, the administering occurs about once per day for one or more days. In embodiments, the administering occurs more than once per day for one or more days. In some embodiments, the administering occurs at most once per day for one or more days. In some embodiments, the administering occurs substantially continuously per administration period.

In some embodiments, for any herein described method, the present disclosure provides a pharmaceutical composition comprising at least one perturbagen that decreases the differentiation to goblet cells, increases the differentiation to ciliated cells without impacting other epithelial lineages.

In some embodiments, for any herein described method, the present disclosure provides a pharmaceutical composition comprising at least one perturbagen that restores differentiation of basal cells to mucus-clearing ciliated cells while simultaneously suppresses differentiation to mucus-secreting goblet cells thereby reduces the total mucus production, which furthermore ameliorates chronic infections and inflammation.

In some embodiments, for any herein described method, the treatment with at least one perturbagen based therapies provide one or more of: (a) decreased secretion of mucus by a goblet cell and (b) decreased synthesis of one or more mucins, optionally selected from MUC5AC, MUC5B, MUC2, MUC4, MUC7, MUC8, and MUC19

### Administration, Dosing, and Treatment Regimens

As examples, administration results in the delivery of one or more perturbagens disclosed herein into the bloodstream (via transdermal, enteral or parenteral administration), or alternatively, the one or more perturbagens is administered by oral inhalation to directly deposit on an area of airway epithelium in the lung.

Delivery of one or more perturbagens disclosed herein into the bloodstream may be via intravenous injection or intravenous infusion or via intraosseous injection or intraosseous infusion. Devices and apparatuses for performing these delivery methods are well known in the art.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In other embodiments, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In other embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In other embodiments, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed.

Any perturbagen disclosed herein can be administered in a single daily dose (also known as QD, qd or q.d.), or the total daily dosage can be administered in divided doses of twice daily (also known as BID, bid, or b.i.d.), three times daily (also known as TID, tid, or t.i.d.), or four times daily (also known as QID, qid, or q.i.d.). Furthermore, any perturbagen disclosed herein can be administered continuously rather than intermittently throughout the dosage regimen.

Any perturbagen disclosed herein can be administered about once per day for one or more days. Any perturbagen disclosed herein can be administered more than once per day for one or more days. Any perturbagen disclosed herein can be administered at most once per day for one or more days. Any perturbagen disclosed herein can be administered substantially continuously per administration period.

### Pharmaceutical Compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use.

P. H. Stahl and C. G. Wermuth (eds.), Verlag, Zurich (Switzerland) 2002, which are hereby incorporated by reference in their entirety. In some embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like.

The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In some embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In some embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions.

Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

The present disclosure includes the disclosed perturbagens in various particle-based formulations suitable for pulmonary airway administration to achieve localized deposit of perturbagen to a desired region of the lung (e.g., lung alveoli).

In some embodiments, the present disclosure provides a pharmaceutical composition comprising aerosolized perturbagen microparticles to treat chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma. In this aspect, the microparticles include nanoparticles having median particle size less than 1000 nm or microparticles having median particle size at 1 micron or greater.

In some embodiments, the herein described aerosolized perturbagen microparticles comprise microparticles having a median particle size greater than 10 microns for delivery to oropharyngeal region of the respiratory system. In some embodiments, the herein described aerosolized perturbagen microparticles comprise microparticles having a median particle size ranging from about 5 microns to about 10 microns for delivery to large conductive airway or oropharyngeal region of the respiratory system. In some embodiments, the herein described aerosolized perturbagen microparticles comprise microparticles having a median particle size ranging from about 1 microns to about 5 microns for delivery to small airways and alveoli of the lung. In some embodiments, the herein described aerosolized perturbagen microparticles comprise microparticles having a median particle size ranging from about 0.1 micron to about 3 microns for delivery to airways, lower airways and alveoli of the lung. In some embodiments, the herein described aerosolized perturbagen microparticles comprise microparticles having a median particle size less than 0.1 micron for delivery to peripheral airways and alveoli of the lung. (Paranjpe et al., Int. J. Mol. Sci., 2014, vol. 15 pp. 5852-5873).

In some embodiments, the herein described aerosolized perturbagen microparticles are used for treating COPD. In this aspect, the aerosolized perturbagen microparticles further include a conventional COPD therapy selected from the group consisting of inhaled β2-agonist including levalbuterol, albuterol, formoterol, foradil, salmeterol, arformoterol; inhaled anticholinergics including ipratropium, triotropium; inhaled corticosteroids including beclomethasone, budesonide, fluticasone, triamcinolone; salmeterol and fluticasone; formoterol and budesonide; theophylline and combinations thereof.

In some embodiments, the herein described aerosolized perturbagen microparticles are used for treating asthma. In this aspect, the aerosolized perturbagen microparticles further include a conventional asthma therapy selected from the group consisting of inhaled β2-agonist including albuterol, formoterol; inhaled anticholinergics including ipratropium; inhaled corticosteroids, inhaled adrenergic agonist including epinephrine; corticosteroids, anti-leukotriene agents including montelukast and zafirlukast; mast cell stabilizers including cromolyn sodium; chloroquine; and combinations thereof.

In some embodiments, the herein described aerosolized perturbagen microparticles are used for treating severe glucocorticoid-resistant asthma. In this aspect, the aerosolized perturbagen microparticles further include a conventional CRS asthma therapy selected from the group consisting of Lactacystine, β-lactone, cyclosporine A, ALLnL (N-acetyl-leucinyl-leucynil-norleucynal, MG101), LLM (N-acetyl-leucinyl-leucynil-methional), Z-LLnV (carbobenzoxyl-leucinyl-leucynil-norvalinal, MG115), Z-LLL (N-carbobenzoxyl-L-leucinyl-L-leucinyl-L-norleucinal, MG132), ubiquitin ligase inhibitors, boronic acid peptide, bortezomib, salinosporamide A, FK506 (tacrolimus), deoxyspergualin, disulfiram, antioxidant, vitamin D3 combined with glucocorticoid, and combinations thereof.

In some embodiments, the herein described aerosolized perturbagen microparticles are used for treating cystic fibrosis.

In this aspect, the aerosolized perturbagen microparticles further include a conventional CF therapy selected from the group consisting of mucolytic agent, hydrating agent, bronchodilator agent, antibiotic, anti-inflammatory drug, pancreatic enzyme, ivacaftor for CF having G551D mutations, lumacaftor and vitamin supplements.

In some embodiments, the herein described aerosolized perturbagen microparticles are prepared from dry powdered material. In some embodiments, the herein described aerosolized perturbagen microparticles are formulated as particle dispersion in a pharmaceutically acceptable liquid medium and/or propellant. In some embodiments, the propellant for aerosolized perturbagen microparticles is selected from the group consisting of hydrofluoroalkane (HFA) including 1,1,1,2-tetrafluoroethane (HFA134a) and 1,1,1,2,3, 3,3-heptafluoropropane (HFA 227); hydrocarbon having a boiling point of −5° C. to +40° C. including n-butane, n-pentane, i-pentane, neopentane; and combinations thereof.

In some embodiments, polymers suitable for preparing the aerosolized perturbagen microparticles may be selected from the group consisting of polyethylene glycol (PEG), poly(N-[2-hydroxypropyl]-methacrylamide), poly(lactide-co-glicolide) (PLGA), polyethylene glycol modified PLGA, poly(ethylene glycol)-poly(ε-caprolactone)-poly(ethylene glycol), monomethoxy poly(ethylene glycol)-poly(ε-caprolactine), poly(ethylene glycol)-b-poly(D, L-lactic acid), mesoporous silica, chitosan, dextran, gelatin, polyethylene glycol modified chitosan, lipid (e.g., phospholipids, phosphatidylcholine, phosphatidylglycerol, phosphatidylethanolamine), dendrimer, protein (e.g., silk, zein protein, whey, sodium caseinate, albumin), elastin-like polypeptide, and combinations thereof.

Where necessary, the pharmaceutical compositions comprising the perturbagens can also include a solubilizing agent. Also, the agents can be delivered with a suitable vehicle or delivery device as known in the art.

In some embodiments, the present disclosure provides a pharmaceutical composition comprising at least one perturbagens selected from Table 4 for the treatment of a disease or disorder selected from the group consisting of chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

In some embodiments, the present disclosure provides a pharmaceutical composition comprising the combination of two or more perturbagens, each with a different mechanism of action, selected from Table 4 for the treatment of a disease or disorder selected from the group consisting of chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma. In some embodiments, the disease or disorder is COPD. In some embodiments, the disease or disorder is asthma.

In some embodiments, the present disclosure provides a pharmaceutical composition comprising the combination of two or more perturbagens, each with a different mechanism of action, selected from Table 4 for the treatment of a disease or disorder selected from the group consisting of chronic obstructive pulmonary disease (COPD), cystic fibrosis (CF), primary ciliary dyskinesia (PCD), non-cystic fibrosis bronchiectasis (NCFB), asthma, and severe glucocorticoid-resistant asthma. In some embodiments, the disease or disorder is COPD. In some embodiments, the disease or disorder is asthma. In some embodiments, the disease or disorder is NCFB. In some embodiments, the disease or disorder is PCD. In some embodiments, the disease or disorder is cystic fibrosis. In some embodiments, the disease or disorder is severe glucocorticoid-resistant asthma.

In some embodiments, two or more perturbagens selected from Table 4, or a variant thereof may be mixed into a single preparation or two or more perturbagens of the combination may be formulated into separate preparations for use in combination separately or at the same time. In some embodiments, the present disclosure provides a kit containing the two or more perturbagens selected from Table 4, or a variant thereof, formulated into separate preparations. In some embodiments, the combination therapies, comprising more than one perturbagen, can be co-delivered in a single delivery vehicle or delivery device.

As used herein, the term “combination” or “pharmaceutical combination” refers to the combined administration of the perturbagens. The combination of two or more perturbagen may be formulated as fixed dose combination or co-packaged discrete perturbagen dosages. In some embodiments, the fixed dose combination therapy of perturbagens comprises bilayer tablet, triple layer tablet, multilayered tablet, or capsule having plurality populations of particles of perturbagens. In some embodiments, the combination of two or more perturbagens may be administered to a subject in need thereof, e.g., concurrently or sequentially.

In some embodiments, the combination therapies of perturbagens as described above give synergistic effects on promoting the proliferation of ciliated cell of airway epithelium of the lung in a subject. The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 40%, 50%, 60%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination (Chou, Cancer Res. 2010, vol. 70, pp. 440-446). When the CI value is less than 1, there is synergy between the compounds used in the combination; when the CI value is equal to 1, there is an additive effect between the compounds used in the combination and when CI value is more than 1, there is an antagonistic effect.

The synergistic effect may be attained by co-formulating the agents of the pharmaceutical combination. The synergistic effect may be attained by administering two or more agents as separate formulations administered simultaneously or sequentially.

Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In some embodiments, any perturbagens disclosed herein is formulated in accordance with routine procedures as a pharmaceutical composition adapted for a mode of administration disclosed herein.

### Other Aspects of the Present Disclosure

Embodiments associated with any of the above-disclosed aspects are likewise relevant to the below-mentioned aspects. In other words, each of the embodiments mentioned above for the above aspects may be revised/adapted to be applicable to the below aspects.

Yet another aspect of the present disclosure is a use of the perturbagen of Table 4, or a variant thereof in the manufacture of a medicament for treating a disease or disorder characterized by an abnormal ratio of the number of goblet cell to the number of one or more of basal cell, basal luminal precursor cell, club cell, ciliated cell, and neuroendocrine cell.

In an aspect, the present disclosure provides a use of the perturbagen of Table 4, or a variant thereof in the manufacture of a medicament for treating a disease or disorder characterized by the number of goblet cells to the number of ciliated cells.

An aspect of the present disclosure is a method for making a therapeutic agent for a disease or disorder selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma. The method comprises (a) identifying a therapeutic agent for therapy according to any of the embodiments disclosed herein, and (b) formulating the candidate perturbation as a therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into ciliated cells or immediate progenitors thereof following exposure of the population of cells to the perturbation; and identifying the perturbation as a candidate perturbation for promoting the transition of a population of progenitor cells into ciliated cells or immediate progenitors thereof based on the perturbation signature. Further, in this aspect, the perturbation signature is an increase in expression and/or activity in the progenitor cell of one or more genes selected from in an activation of a network module designated in the network module column of Table 3. In some embodiments, the activation of one or more genes of the network module designated in the network module column of Table 3 comprises modulating expression and/or activity of 2, 3, 4, 5, 6 7, 8, 9, 10 or more genes within a network module. In some embodiments, altering the gene signature comprises a decrease in expression and/or activity in the progenitor cell of one or more genes designated as a “down” gene in the gene directionality column of Table 3. In some embodiments, altering the gene signature comprises a decrease in expression and/or activity in the progenitor cell of two or more genes designated as a “down” gene in the gene directionality column of Table 3.

In some embodiments, the present disclosure is related to a method for making a therapeutic agent for a disease or disorder characterized by an abnormal ratio of goblet cells to basal luminal cells, club cells, ciliated cells, and/or neuroendocrine cells. This method includes the steps of (a) identifying a candidate perturbation according to herein described method; and (b) formulating the candidate perturbation as a therapeutic agent for the treatment of the disease or disorder. In some embodiments, the present disclosure is related to a method for making a therapeutic agent for a disease or disorder characterized by an abnormal ratio of goblet cells to basal cells, club cells, ciliated cells, and/or neuroendocrine cells. This method includes the steps of (a) identifying a candidate perturbation according to herein described method; and (b) formulating the candidate perturbation as a therapeutic agent for the treatment of the disease or disorder.

Yet another aspect of the present disclosure is a perturbagen capable of causing a change in a gene signature.

In an aspect, the present disclosure provides a perturbagen capable of causing a change in cell fate.

In another aspect, the present disclosure provides a perturbagen capable of causing a change in a gene signature and a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

The instant disclosure also provides certain embodiments as follows:

Embodiment 1001: A method for inhibiting a change in cell state of a progenitor cell comprising: contacting a population of cells comprising a progenitor cell with at least one perturbagen selected from Table 4, or a variant thereof, wherein the at least one perturbagen is capable of altering a gene signature in the progenitor cell; and wherein the progenitor cell is a basal cell.

Embodiment 1002: A method for inhibiting a change in cell state of a progenitor cell, comprising: contacting a population of cells comprising a progenitor cell with at least one perturbagen capable of altering a gene signature in the progenitor cell, wherein altering the gene signature comprises a decrease in expression and/or activity in the progenitor cell of one or more genes designated as a “down” gene in the gene directionality column of Table 3 and wherein the progenitor cell is a basal cell.

Embodiment 1003: A method for inhibiting a change in cell state of a progenitor cell, comprising: contacting a population of cells comprising a progenitor cell with at least one perturbagen selected from Table 4, or a variant thereof, and capable of altering a gene signature in the progenitor cell, wherein altering the gene signature comprises a decrease in expression and/or activity in the progenitor cell of one or more genes selected from Table 4 and wherein the progenitor cell is a basal cell.

Embodiment 1004: The method of any one of Embodiments 1001-1003, wherein altering the gene signature comprises an activation of a network module designated in the network module column of Table 3.

Embodiment 1005: The method of Embodiment 1004, wherein the activation of the network module designated in the network module column of Table 3 comprises modulating expression and/or activity of 2 or more genes within a network module.

Embodiment 1006: The method of Embodiment 1005, wherein the activation of the network module designated in the network module column of Table 3 comprises modulating expression and/or activity of all of the genes within a network module.

Embodiment 1007: The method of any one of Embodiments 1001-1003, wherein the activation of the network module designated in the network module column of Table 3 comprises modulating expression and/or activity of 2 or more genes within 2 or more network modules.

Embodiment 1008: The method of any one of Embodiments 1001-1003, wherein inhibiting the change in cell state provides: i) a decrease in the number of goblet cells; ii) an increase in the number of club cells; and/or iii) an increase in the number of ciliated cells.

Embodiment 1009: The method of Embodiment 1008, wherein inhibiting the change in cell state provides a decrease in the number of goblet cells.

Embodiment 1010: The method of any of Embodiments 1008-1009, wherein inhibiting the change in cell state provides an increase in the number of club cells.

Embodiment 1011: The method of any of Embodiments 1008-1009, wherein inhibiting the change in cell state provides an increase in the number of ciliated cells.

Embodiment 1012: The method of Embodiment 1008, wherein inhibiting the change in cell state provides an increase in the number of ciliated cells and a decrease in the number of goblet cells.

Embodiment 1013: The method of Embodiment 1008, wherein the decrease in the number of goblet cells is relative to the number of basal cells obtained from a population of progenitor cells i) that is not contacted with the at least one perturbagen or ii) prior to contacting with the at least one perturbagen.

Embodiment 1014: The method of Embodiment 1008, wherein the decrease in the number of goblet cells is relative to the number of basal luminal precursor cells obtained from a population of progenitor cells i) that is not contacted with the at least one perturbagen or ii) prior to contacting with the at least one perturbagen.

Embodiment 1015: The method of Embodiment 1008, wherein the decrease in the number of goblet cells is relative to the number of club cells obtained from a population of progenitor cells i) that is not contacted with the at least one perturbagen or ii) prior to contacting with the at least one perturbagen.

Embodiment 1016: The method of Embodiment 1008, wherein the decrease in the number of goblet cells is relative to the number of ciliated cells obtained from a population of progenitor cells i) that is not contacted with the at least one perturbagen or ii) prior to contacting with the at least one perturbagen.

Embodiment 1017: The method of Embodiment 1008, wherein the decrease in the number of goblet cells is relative to the number of neuroendocrine cells obtained from a population of progenitor cells i) that is not contacted with the at least one perturbagen or ii) prior to contacting with the at least one perturbagen.

Embodiment 1018: The method of Embodiment 1008, wherein the increase in the number of club cells is relative to the number of basal cells, goblet cells, basal luminal precursor cells, ciliated cells, and/or neuroendocrine cells obtained from a population of progenitor cells i) that is not contacted with the at least one perturbagen or ii) prior to contacting with the at least one perturbagen.

Embodiment 1019: The method of Embodiment 1008, wherein the increase in the number of ciliated cells is relative to the number of basal cells, goblet cells, basal luminal precursor cells, club cells, and/or neuroendocrine cells obtained from a population of progenitor cells i) that is not contacted with the at least one perturbagen or ii) prior to contacting with the at least one perturbagen.

Embodiment 1020: The method of Embodiments 1001-1019, wherein inhibiting the change in cell state does not provide a substantial increase in the number of goblet cells.

Embodiment 1021: The method of Embodiment 1008, wherein the ratio of the number of goblet cells to the number of basal cells is decreased relative to the ratio obtained from a population of progenitor cells that is not contacted with the at least one perturbagen.

Embodiment 1022: The method of Embodiment 1008, wherein the ratio of the number of goblet cells to the number of basal cells is decreased relative to the ratio in the population of progenitor cells prior to contacting with the at least one perturbagen.

Embodiment 1023: The method of Embodiment 1008, wherein the ratio of the number of goblet cells to the number of basal luminal precursor cells is decreased relative to the ratio obtained from a population of progenitor cells that is not contacted with the at least one perturbagen.

Embodiment 1024: The method of Embodiment 1008, wherein the ratio of the number of goblet cells to the number of basal luminal precursor cells is decreased relative to the ratio in the population of progenitor cells prior to contacting with the at least one perturbagen.

Embodiment 1025: The method of Embodiment 1008, wherein the ratio of the number of goblet cells to the number of club cells is decreased relative to the ratio obtained from a population of progenitor cells that is not contacted with the at least one perturbagen.

Embodiment 1026: The method of Embodiment 1008, wherein the ratio of the number of goblet cells to the number of club cells is decreased relative to the ratio in the population of progenitor cells prior to contacting with the at least one perturbagen.

Embodiment 1027: The method of Embodiment 1008, wherein the ratio of the number of goblet cells to the number of ciliated cells is decreased relative to the ratio obtained from a population of progenitor cells that is not contacted with the at least one perturbagen.

Embodiment 1028: The method of Embodiment 1008, wherein the ratio of the number of goblet cells to the number of ciliated cells is decreased relative to the ratio in the population of progenitor cells prior to contacting with the at least one perturbagen.

Embodiment 1029: The method of Embodiment 1008, wherein the ratio of the number of goblet cells to the number of neuroendocrine cells is decreased relative to the ratio obtained from a population of progenitor cells that is not contacted with the at least one perturbagen.

Embodiment 1030: The method of Embodiment 1008, wherein the ratio of the number of goblet cells to the number of neuroendocrine cells is decreased relative to the ratio in the population of progenitor cells prior to contacting with the at least one perturbagen.

Embodiment 1031: The method of Embodiment 1008, wherein the ratio of the number of club cells to the number of basal cells is increased relative to the ratio obtained from a population of progenitor cells that is not contacted with the at least one perturbagen.

Embodiment 1032: The method of Embodiment 1008, wherein the ratio of the number of club cells to the number of basal cells is increased relative to the ratio in the population of progenitor cells prior to contacting with the at least one perturbagen.

Embodiment 1033: The method of Embodiment 1008, wherein the ratio of the number of club cells to the number of basal luminal precursor cells is increased relative to the ratio obtained from a population of progenitor cells that is not contacted with the at least one perturbagen.

Embodiment 1034: The method of Embodiment 1008, wherein the ratio of the number of club cells to the number of basal luminal precursor cells is increased relative to the ratio in the population of progenitor cells prior to contacting with the at least one perturbagen.

Embodiment 1035: The method of Embodiment 1008, wherein the ratio of the number of club cells to the number of ciliated cells is increased relative to the ratio obtained from a population of progenitor cells that is not contacted with the at least one perturbagen.

Embodiment 1036: The method of Embodiment 1008, wherein the ratio of the number of club cells to the number of ciliated cells is increased relative to the ratio in the population of progenitor cells prior to contacting with the at least one perturbagen.

Embodiment 1037: The method of Embodiment 1008, wherein the ratio of the number of club cells to the number of neuroendocrine cells is increased relative to the ratio obtained from a population of progenitor cells that is not contacted with the at least one perturbagen.

Embodiment 1038: The method of Embodiment 1008, wherein the ratio of the number of club cells to the number of neuroendocrine cells is increased relative to the ratio in the population of progenitor cells prior to contacting with the at least one perturbagen.

Embodiment 1039: The method of Embodiment 1008, wherein the ratio of the number of club cells to the number of goblet cells is increased relative to the ratio obtained from a population of progenitor cells that is not contacted with the at least one perturbagen.

Embodiment 1040: The method of Embodiment 1008, wherein the ratio of the number of club cells to the number of goblet cells is increased relative to the ratio in the population of progenitor cells prior to contacting with the at least one perturbagen.

Embodiment 1041: The method of Embodiment 1008, wherein the ratio of the number of ciliated cells to the number of basal cells is increased relative to the ratio obtained from a population of progenitor cells that is not contacted with the at least one perturbagen.

Embodiment 1042: The method of Embodiment 1008, wherein the ratio of the number of ciliated cells to the number of basal cells is increased relative to the ratio in the population of progenitor cells prior to contacting with the at least one perturbagen.

Embodiment 1043: The method of Embodiment 1008, wherein the ratio of the number of ciliated cells to the number of basal luminal precursor cells is increased relative to the ratio obtained from a population of progenitor cells that is not contacted with the at least one perturbagen.

Embodiment 1044: The method of Embodiment 1008, wherein the ratio of the number of ciliated cells to the number of basal luminal precursor cells is increased relative to the ratio in the population of progenitor cells prior to contacting with the at least one perturbagen.

Embodiment 1045: The method of Embodiment 1008, wherein the ratio of the number of ciliated cells to the number of club cells is increased relative to the ratio obtained from a population of progenitor cells that is not contacted with the at least one perturbagen.

Embodiment 1046: The method of Embodiment 1008, wherein the ratio of the number of ciliated cells to the number of club cells is increased relative to the ratio in the population of progenitor cells prior to contacting with the at least one perturbagen.

Embodiment 1047: The method of Embodiment 1008, wherein the ratio of the number of ciliated cells to the number of goblet cells is increased relative to the ratio obtained from a population of progenitor cells that is not contacted with the at least one perturbagen.

Embodiment 1048: The method of Embodiment 1008, wherein the ratio of the number of ciliated cells to the number of goblet cells is increased relative to the ratio in the population of progenitor cells prior to contacting with the at least one perturbagen.

Embodiment 1049: The method of any one of Embodiments 1001-1048, wherein inhibiting the change in cell state does not provide a substantial increase in the number of goblet cells.

Embodiment 1050: The method of any one of Embodiments 1001-1020, wherein the decrease in the number of goblet cells is due in part to decreased cell proliferation of the goblet cells, basal luminal precursor cells, and/or club cells.

Embodiment 1051: The method of any one of Embodiments 1001-1020, wherein the decrease in the number of goblet cells is due in part to a decreased lifespan of the goblet cells, basal luminal precursor cells, and/or club cells.

Embodiment 1052: The method of any one of Embodiments 1001-1020, wherein the decrease in the number of goblet cells is due in part to increased cell death among the goblet cells, basal luminal precursor cells, and/or club cells.

Embodiment 1053: The method of any one of Embodiments 1001-1020, wherein the decrease in the number of goblet cells is due in part to blocking the progression from: i) basal cell to basal luminal precursor cell; ii) basal luminal precursor cell to club cell; and/or iii) club cell to goblet cell.

Embodiment 1054: The method of any one of Embodiments 1001-1008, wherein the number of basal cells is increased.

Embodiment 1055: The method of Embodiment 1054, wherein the increase in the number of basal cells is due in part to i) increased cell proliferation of the basal cells; ii) an increased lifespan of the basal cells; and/or iii) decreased cell death among the basal cells.

Embodiment 1056: The method of any one of Embodiments 1053-1055, wherein the increase in the number of basal cells is relative to the number of basal cells in a population of basal cells that is not contacted with the at least one perturbagen.

Embodiment 1057: The method of any one of Embodiments 1053-1055, wherein the increase in the number of basal cells is relative to the number of basal cells in the population prior to contacting with the at least one perturbagen.

Embodiment 1058: The method of any one of Embodiments Embodiment 1053-1057, wherein the increase in the number of basal cells is due to inhibiting a change of cell state from a basal cell into the goblet cell lineage and/or club cell lineage.

Embodiment 1059: The method of any one of Embodiments 1053-1058, wherein the number of basal luminal precursor cells, and/or goblet cells is decreased after contacting the population of cells comprising a basal cell with the at least one perturbagen.

Embodiment 1060: The method of Embodiment 1059, wherein the ratio of the number of basal luminal precursor cells, and/or goblet cells, to the number of basal cells is decreased relative to the ratio obtained from a population of progenitor cells that is not contacted with the at least one perturbagen.

Embodiment 1061: The method of Embodiment 1059, wherein the ratio of the number of basal luminal precursor cells, and/or goblet cells to the number of basal cells is decreased relative to the ratio in the population of progenitor cells prior to contacting with the at least one perturbagen.

Embodiment 1062: The method of any one of Embodiments 1001-1061, wherein the at least one perturbagen selected from Table 4, or a variant thereof, comprises at least 2, at least 3, at least 4, at least 5, at least 6, at least 7, at least 8, at least 9, at least 10, at least 11, at least 12, or 13 perturbagens selected from Table 4, or variants thereof.

Embodiment 1063: The method of any one of Embodiments 1001-1062, wherein altering the gene signature comprises decreased expression and/or decreased activity in the progenitor cell of one or more genes designated as a “down” gene in the gene directionality column of Table 3.

Embodiment 1064: The method of Embodiment 1063, wherein the one or more genes selected from Table 3 comprises 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more, or 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, 67 or more, or 68 or more genes designated as a “down” gene in the gene directionality column of Table 3.

Embodiment 1065: The method of Embodiment 1063 or 1064, wherein the one or more genes designated as a “down” gene in the gene directionality column of Table 3 are selected from PLP2, GAPDH, SNCA, CDH3, FKBP4, CAMSAP2, PPP1R13B, NISCH, HTRA1, ATP11B, ETS1, CPSF4, TLE1, CDK2, SESN1, GRB7, CERK, ZNF318, MYC, ELOVL6, STAMBP, EBNA1BP2, MSH6, FAH, EIF4EBP1, SLC35F2, RRP1B, G3BP1, UTP14A, DUSP3, FHL2, VPS72, ARL4C, ARPP19, CDKN1B, TP53, CRYZ, PLOD3, DDIT4, LAMA3, INPP1, CDK7, KLHL21, TIAM1, TIPARP, FOXJ3, NPC1, TUBB6, TPM1, RPA3, SFN, ST3GAL5, GMNN, ACOT9, BLMH, NNT, USP1, FKBP14, HSPB1, TBP, EPB41L2, CDCA4, TRAM2, CETN3, ETRN, PDLIM1, BRCA1, and LOXL1.

Embodiment 1066: The method of any one of Embodiments 1001-1065, wherein contacting the population of cells comprising a progenitor cell occurs in vitro or ex vivo.

Embodiment 1067: The method of any one of Embodiments 1001-1065, wherein contacting the population of cells comprising a progenitor cell occurs in vivo in a subject.

Embodiment 1068: The method of Embodiment 1067, wherein the subject is a human.

Embodiment 1069: The method of Embodiment 1068, wherein the human is an adult human.

Embodiment 1070: A perturbagen for use in the method of any one of Embodiments 1001-1069.

Embodiment 1071: A pharmaceutical composition comprising the perturbagen of Embodiment 1070.

Embodiment 1072: The method of any one of Embodiments 1001-1069, wherein the change in cells state provides one or more of: (a) decreased secretion of mucus by a goblet cell and (b) decreased synthesis of one or more mucins, optionally selected from MUC5AC, MUC5B, MUC2, MUC4, MUC7, MUC8, and MUC19.

Embodiment 1073: A method for inhibiting the formation of a goblet cell or an immediate progenitor thereof, comprising: exposing a starting population of progenitor cells comprising at least one basal cell to a perturbation having a perturbation signature that prevents progression of a progenitor cell into and/or reduces the likelihood that a progenitor cell will progress into a goblet cell or other lineage associated progenitor thereof, wherein the perturbation signature comprises a decreased expression and/or activity in the progenitor cells of one or more genes selected from Table 3 designated as a “down” gene in the gene directionality column of Table 3.

Embodiment 1074: The method of Embodiment 1073, wherein the formation of goblet cells is inhibited.

Embodiment 1075: The method of Embodiment 1073, wherein the perturbation signature comprises an activation of a network module designated in the network module column of Table 3.

Embodiment 1076: The method of Embodiment 1075, wherein the activation of the network module designated in the network module column of Table 3 comprises modulating expression and/or activity of 2 or more genes within a network module.

Embodiment 1077: The method of Embodiment 1076, wherein the activation of the network module designated in the network module column of Table 3 comprises modulating expression and/or activity of all of the genes within a network module.

Embodiment 1078: The method of Embodiment 1073, wherein the activation of the network module designated in the network module column of Table 3 comprises modulating expression and/or activity of 2 or more genes within 2 or more network modules.

Embodiment 1079: A method for promoting the formation of a ciliated cell, or an immediate progenitor thereof, comprising: exposing a starting population of progenitor cells comprising at least one basal cell to a perturbation having a perturbation signature that promotes progression of a progenitor cell into and/or increases the likelihood that a progenitor cell will progress into a ciliated cell or other lineage associated progenitor thereof, wherein the perturbation signature comprises a decreased expression and/or activity in the progenitor cells of one or more genes selected from designated as a “down” gene in the gene directionality column of Table 3.

Embodiment 1080: The method of Embodiment 1079, wherein the perturbation signature comprises an activation of a network module designated in the network module column of Table 3.

Embodiment 1081: The method of Embodiment 1080, wherein the activation of the network module designated in the network module column of Table 3 comprises modulating expression and/or activity of 2 or more genes within a network module.

Embodiment 1082: The method of Embodiment 1081, wherein the activation of the network module designated in the network module column of Table 3 comprises modulating expression and/or activity of all of the genes within a network module.

Embodiment 1083: The method of Embodiment 1079, wherein the activation of the network module designated in the network module column of Table 3 comprises modulating expression and/or activity of 2 or more genes within 2 or more network modules.

Embodiment 1084: A method for treating a disease or disorder characterized by an abnormal number or abnormal function of goblet cells, comprising: (a) administering to a patient in need thereof a therapeutically effective amount of at least one perturbagen selected from Table 4, or a variant thereof, wherein the at least one perturbagen is capable of changing a gene signature in a progenitor cell or (b) administering to a patient in need thereof a cell, the cell having been contacted with at least one perturbagen selected from Table 4, or a variant thereof, wherein the at least one perturbagen is capable of changing a gene signature in a progenitor cell.

Embodiment 1085: The method of Embodiment 1084, wherein the disease or disorder is caused by an increase in the number of goblet cells or an increase in the production of mucus by goblet cells.

Embodiment 1086: The method of Embodiment 1084, wherein the administering is via intraosseous injection or intraosseous infusion.

Embodiment 1087: The method of Embodiment 1084, wherein the administering the cell is via intravenous injection or intravenous infusion.

Embodiment 1088: The method of Embodiment 1084, wherein the administering is simultaneously or sequentially to one or more mobilization agents.

Embodiment 1089: The method of Embodiment 1084, wherein the administering of the perturbagen is via respiratory tract, oral, intravenous, intramuscular, intraperitoneal, subcutaneous, intra-articular injection, and/or infusion route.

Embodiment 1090: The method of Embodiment 1084, wherein the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

Embodiment 1091: The method of Embodiment 1084, wherein at least one perturbagen is administered on the basis of previously determining that the patient exhibits an abnormal number of goblet cells, or a disease or disorder characterized thereby.

Embodiment 1092: The method of Embodiment 1084, wherein at least one perturbagen is administered on the basis of previously determining that the patient exhibits an abnormal function of goblet cells, or a disease or disorder characterized thereby.

Embodiment 1093: The method of Embodiment 1092, wherein the abnormal function of goblet cells includes increase in the production of mucus by the goblet cells.

Embodiment 1094: A method for treating a disease or disorder characterized by an abnormal ratio of goblet cells to basal cells, comprising: (a) administering to a patient in need thereof at least one perturbagen selected from Table 4, or a variant thereof, wherein the at least one perturbagen is capable of changing a gene signature in a basal cell or (b) administering to a patient in need thereof a cell, the cell having been contacted with at least one perturbagen selected from Table 4, or a variant thereof, wherein the at least one perturbagen is capable of changing a gene signature in a basal cell.

Embodiment 1095: The method of Embodiment 1094, wherein the abnormal ratio comprises a decreased number of goblet cells and/or an increased number of basal cells.

Embodiment 1096: The method of Embodiment 1095, wherein the abnormal ratio comprises an increased number of basal cells.

Embodiment 1097: The method of Embodiment 1095, wherein the abnormal ratio comprises a decreased number of goblet cells.

Embodiment 1098: The method of Embodiment 1094, wherein the administering is via intraosseous injection or intraosseous infusion.

Embodiment 1099: The method of Embodiment 1094, wherein the administering the cell is via intravenous injection or intravenous infusion.

Embodiment 1100: The method of Embodiment 1094, wherein the administering is simultaneously or sequentially to one or more mobilization agents.

Embodiment 1101: The method of Embodiment 1094, wherein the administering of the perturbagen is via respiratory tract, oral, intravenous, intramuscular, intraperitoneal, subcutaneous, intra-articular injection, and/or infusion route.

Embodiment 1102: The method of Embodiment 1094, wherein the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

Embodiment 1103: The method of Embodiment 1094, wherein at least one perturbagen is administered on the basis of previously determining that the patient exhibits the abnormal ratio of goblet cells to basal cells, or a disease or disorder characterized thereby.

Embodiment 1104: The method of any one of Embodiments 1094-1103, wherein the at least one perturbagen is capable of changing a gene signature in a basal cell.

Embodiment 1105: The method of any one of Embodiments 1094-1104, wherein the patient is selected by steps comprising: obtaining from the patient having the disease or disorder a sample of cells comprising at least one basal cell; and contacting the sample of cells with least one perturbagen selected from Table 4, or a variant thereof, wherein the at least one perturbagen alters a gene signature in the sample of cells.

Embodiment 1106: The method of any one of Embodiments 1094-1104, wherein the patient is selected by steps comprising: obtaining from a subject having the disease or disorder a sample of cells comprising at least one basal cell; and contacting the sample of cells with at least one perturbagen capable of altering a gene signature in a basal cell, wherein the at least one perturbagen decreases in the sample of cells the expression and/or activity of one or more genes selected from Table 3 designated as a “down” gene in the gene directionality column of Table 3.

Embodiment 1107: The method of Embodiment 1106, wherein the perturbation signature comprises an activation of a network module designated in the network module column of Table 3.

Embodiment 1108: The method of Embodiment 1107, wherein the activation of the network module designated in the network module column of Table 3 comprises modulating expression and/or activity of 2 or more genes within a network module.

Embodiment 1109: The method of Embodiment 1108, wherein the activation of the network module designated in the network module column of Table 3 comprises modulating expression and/or activity of all of the genes within a network module.

Embodiment 1110: The method of Embodiment 1106, wherein the activation of the network module designated in the network module column of Table 3 comprises modulating expression and/or activity of 2 or more genes within 2 or more network modules.

Embodiment 1111: The method of any one of Embodiments 1094-1104, wherein the patient is selected by steps comprising: obtaining from a subject having the disease or disorder a sample of cells comprising a basal cell; and contacting the sample of cells with at least one perturbagen selected from Table 4, or a variant thereof; wherein the at least one perturbagen decreases in the sample of cells the expression and/or activity of one or more genes selected from Table 3 designated as a “down” gene in the gene directionality column of Table 3.

Embodiment 1112: The method of Embodiment 1111, wherein the method alters a gene signature in the sample of cells, comprising activation of a network module designated in the network module column of Table 3.

Embodiment 1113: The method of Embodiment 1112, wherein the activation of the network module designated in the network module column of Table 3 comprises modulating expression and/or activity of 2 or more genes within a network module.

Embodiment 1114: The method of Embodiment 1113, wherein the activation of the network module designated in the network module column of Table 3 comprises modulating expression and/or activity of all of the genes within a network module.

Embodiment 1115: The method of Embodiment 1111, wherein the activation of the network module designated in the network module column of Table 3 comprises modulating expression and/or activity of 2 or more genes within 2 or more network modules.

Embodiment 1116: A method for selecting the patient of any one of Embodiments 1094-1104, comprising: obtaining from a subject having the disease or disorder a sample of cells comprising a basal cell; and contacting the sample of cells with least one perturbagen selected from Table 4, or a variant thereof, wherein when the at least one perturbagen alters a gene signature in the sample of cells, the subject is selected as a patient.

Embodiment 1117: A method for selecting the patient of any one of Embodiments 1094-1104, comprising: obtaining from a subject having the disease or disorder a sample of cells comprising a basal cell; and contacting the sample of cells with at least one perturbagen capable of altering a gene signature in a basal cell, wherein when the at least one perturbagen decreases in the sample of cells the expression and/or activity of one or more genes selected from Table 3 designated as a “down” gene in the gene directionality column of Table 3, the subject is selected as a patient.

Embodiment 1118: The method of Embodiment 1117, wherein the method alters a gene signature in the sample of cells, comprising activation of a network module designated in the network module column of Table 3.

Embodiment 1119: The method of Embodiment 1118, wherein the activation of the network module designated in the network module column of Table 3 comprises modulating expression and/or activity of 2 or more genes within a network module.

Embodiment 1120: The method of Embodiment 1119, wherein the activation of the network module designated in the network module column of Table 3 comprises modulating expression and/or activity of all of the genes within a network module.

Embodiment 1121: The method of Embodiment 1117, wherein the activation of the network module designated in the network module column of Table 3 comprises modulating expression and/or activity of 2 or more genes within 2 or more network modules.

Embodiment 1122: A method for selecting the patient of any one of Embodiments 1094-1104, comprising: obtaining from a subject having the disease or disorder a sample of cells comprising a basal cell; and contacting the sample of cells with at least one perturbagen selected from Table 4, or a variant thereof; wherein when the at least one perturbagen decreases in the sample of cells the expression and/or activity of one or more genes selected from Table 3 designated as a “down” gene in the gene directionality column of Table 3, the subject is selected as a patient.

Embodiment 1123: The method of Embodiment 1122, wherein the method alters a gene signature in the sample of cells, comprising activation of a network module designated in the network module column of Table 3.

Embodiment 1124: The method of Embodiment 1123, wherein the activation of the network module designated in the network module column of Table 3 comprises modulating expression and/or activity of 2 or more genes within a network module.

Embodiment 1125: The method of Embodiment 1124, wherein the activation of the network module designated in the network module column of Table 3 comprises modulating expression and/or activity of all of the genes within a network module.

Embodiment 1126: The method of Embodiment 1122, wherein the activation of the network module designated in the network module column of Table 3 comprises modulating expression and/or activity of 2 or more genes within 2 or more network modules.

Embodiment 1127: Use of the perturbagen of Table 4, or a variant thereof in the manufacture of a medicament for treating a disease or disorder characterized by an abnormal ratio of goblet cells to basal cells.

Embodiment 1128: Use of the perturbagen of Table 4, or a variant thereof in the manufacture of a medicament for treating a disease or disorder characterized by an abnormal ratio of goblet cells to basal luminal cells, club cells, ciliated cells, and/or neuroendocrine cells.

Embodiment 1129: A method of identifying a candidate perturbation for promoting the transition of a starting population of basal cells into goblet cells or immediate progenitors thereof, the method comprising: exposing the starting population of basal cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of basal cells into goblet cells or immediate progenitors thereof following exposure of the population of cells to the perturbation; and identifying the perturbation as a candidate perturbation for promoting the transition of a population of basal cells into goblet cells or immediate progenitors thereof based on the perturbation signature, wherein the perturbation signature is a decrease in expression and/or activity in the basal cell of one or more genes selected from Table 3 designated as a “down” gene in the gene directionality column of Table 3.

Embodiment 1130: The method of Embodiment 1129, wherein the perturbation signature comprises an activation of a network module designated in the network module column of Table 3.

Embodiment 1131: The method of any one of Embodiments 1129-1130, wherein the activation of the network module designated in the network module column of Table 3 comprises modulating expression and/or activity of 2 or more genes within a network module.

Embodiment 1132: The method of any one of Embodiments 1129-1131, wherein the activation of the network module designated in the network module column of Table 3 comprises modulating expression and/or activity of all of the genes within a network module.

Embodiment 1133: The method of any one of Embodiments 1129-1132, wherein the activation of the network module designated in the network module column of Table 3 comprises modulating expression and/or activity of 2 or more genes within 2 or more network modules.

Embodiment 1134: A method for making a therapeutic agent for a disease or disorder characterized by an abnormal ratio of goblet cells to basal luminal cells, club cells, ciliated cells, and/or neuroendocrine cells, comprising: (a) identifying a candidate perturbation according to the method of Embodiment 1129; and (b) formulating the candidate perturbation as a therapeutic agent for the treatment of the disease or disorder.

### Methods of Culturing Cells In Vitro to Perform Single-Cell Analyses

In carrying out the techniques described herein for identifying the causes of cell fate, it is useful to generate datasets regarding cellular-component measurements obtained from single-cells. To generate these datasets, a population of cells of interest may be cultured in vitro. Alternately, these datasets may be generated, from single cells that have not been previously cultured; for example, cells used in single cell analyses may be obtained from dissociated primary tissue or from a blood product. This latter method of generating datasets is often desirable if one wants to capture information of the primary cell/organ as close to the in vivo setting as possible. However, for cells undergoing culturing, single-cell measurements of one or more cellular-components of interest may be performed at one or more time periods during the culturing to generate datasets.

In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof. In some embodiments, the cellular-component measurements comprise gene expression measurements, such as RNA levels.

Any one of a number of single-cell cellular-component expression measurement techniques may be used to collect the datasets. Examples include, but are not limited to single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and so on. The cellular-component expression measurement can be selected based on the desired cellular-component to be measured. For instance, scRNA-seq, scTag-seq, and miRNA-seq measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq measure protein expression in the cell. CITE-seq simultaneously measures both gene expression and protein expression in the cell. And scATAC-seq measures chromatin conformation in the cell. Table 3 below provides links to example protocols for performing each of the single-cell cellular-component expression measurement techniques described herein.

TABLE 3
Example Measurement Protocols

| Technique | Protocol |
|---|---|
| RNA-seq | Olsen and Baryawno “Introduction to Single-Cell RNA Sequencing” Current |
| | Protocols in Molecular Biology. Volume 122, Issue 1, April 2018, e57 |
| Tag-seq | Rozenberg et al., “Digital gene expression analysis with sample multiplexing and |
| | PCR duplicate detection: A straightforward protocol”, BioTechniques, vol. 61, |
| | No. 1, March 2018 |
| ATAC-seq | Buenrostro et al., “ATAC-seq: A Method for Assaying Chromatin Accessibility |
| | Genome-Wide”, Curr Protoc Mol Biol. 2015; 109: 21.29.1-21.29.9 |
| miRNA-seq | Faridani et al., “Single-cell sequencing of the small-RNA transcriptome” Nature |
| | Biotechnology volume 34, pages 1264-1266 (2016) |
| CyTOF/SCoPE-MS/Abseq | Bandura et al., “Mass Cytometry: Technique for Real Time Single Cell |
| | Multitarget Immunoassay Based on Inductively Coupled Plasma Time-of-Flight |
| | Mass Spectrometry”, Anal Chem. 2009 Aug. 15; 81(16): 6813-22 |
| | Shahi et al., “Abseq: Ultrahigh-throughput single cell protein profiling with droplet |
| | microfluidic barcoding”, Scientific Reports volume 7, Article number: 44447 |
| | (2017) |
| | Budnik et al., “SCoPE-MS: mass spectrometry of single mammalian cells |
| | quantifies proteome heterogeneity during cell differentiation”, Genome Biology |
| | 2018 19: 161 |
| CITE-seq | Stoeckius et al., “Simultaneous epitope and transcriptome measurement in |
| | single cells”, Nature Methods, vol 14, pages 865-868 (2017) |

The cellular-component expression measurement technique used may result in cell death. Alternatively, cellular-components may be measured by extracting out of the live cell, for example by extracting cell cytoplasm without killing the cell. Techniques of this variety allow the same cell to be measured at multiple different points in time.

If the cell population is heterogeneous such that multiple different cell types that originate from a same “progenitor” cell are present in the population, then single-cell cellular-component expression measurements can be performed at a single time point or at relatively few time points as the cells grow in culture. As a result of the heterogeneity of the cell population, the collected datasets will represent cells of various types along a trajectory of transition.

If the cell population is substantially homogeneous such that only a single or relatively few cell types, mostly the “progenitor” cell of interest, are present in the population, then single-cell cellular-component expression measurements can be performed multiple times over a period of time as the cells transition.

A separate single-cell cellular-component expression dataset is generated for each cell, and where applicable at each of the time periods. The collection of single-cell cellular-component expression measurements from a population of cells at multiple different points in time can collectively be interpreted as a “pseudo-time” representation of cell expression over time for the cell types originating from the same “progenitor” cell. The term pseudo-time is used in two respects, first, in that cell state transition is not necessarily the same from cell to cell, and thus the population of cell provides a distribution of what transition processes a cell of that “progenitor” type is likely to go through over time, and second, that the cellular-component expression measurements of those multiple cell's expressions at multiple time points simulates the possible transition behavior over time, even if cellular-component expression measurements of distinct cells give rise to the datasets. As a deliberately simple example, even if cell X gave a dataset for time point A and cell Y gave a dataset for time point B, together these two datasets represent the pseudo-time of transition between time point A and time point B.

For convenience of description, two such datasets captured for a “same” cell at two different time periods (assuming a technique is used that does not kill the cell) are herein referred to as different “cells” (and corresponding different datasets) because in practice such cells will often be slightly or significantly transitioned from each other, in some cases having an entirely distinct cell type as determined from the relative quantities of various cellular-components. Viewed from this context, these two measurements of a single-cell at different time points can be interpreted as different cells for the purpose of analysis because the cell itself has changed.

Note that the separation of datasets by cell/time period described herein is for clarity of description, in practice, these datasets may be stored in computer memory and logically operated on as one or more aggregate dataset/s (e.g., by cell for all time periods, for all cells and time periods at once).

In some instances, it is useful to collect datasets where a “progenitor” cell of interest has been perturbed from its base line state. There are a number of possible reasons to do this, for example, to knock out one or more cellular-components, to evaluate the difference between healthy and diseased cell states. In these instances, a process may also include steps for introducing the desired modifications to the cells. For example, one or more perturbations may be introduced to the cells, tailored viruses designed to knock out one or more cellular-components may be introduced, clustered regularly interspaced short palindromic repeats (CRISPR) may be used to edit cellular-components, and so on. Examples of techniques that could be used include, but are not limited to, RNA interference (RNAi), Transcription activator-like effector nuclease (TALEN) or Zinc Finger Nuclease (ZFN).

Depending upon how the perturbation is applied, not all cells will be perturbed in the same way. For example, if a virus is introduced to knockout a particular gene, that virus may not affect all cells in the population. More generally, this property can be used advantageously to evaluate the effect of many different perturbations with respect to a single population. For example, a large number of tailored viruses may be introduced, each of which performs a different perturbation such as causing a different gene to be knocked out. The viruses will variously infect some subset of the various cells, knocking out the gene of interest. Single-cell sequencing or another technique can then be used to identify which viruses affected which cells. The resulting differing single-cell sequencing datasets can then be evaluated to identify the effect of gene knockout on gene expression in accordance with the methods described elsewhere in this description.

Other types of multi-perturbation cell modifications can be performed similarly, such as the introduction of multiple different perturbations, barcoding CRISPR, etc. Further, more than one type perturbation may be introduced into a population of cells to be analyzed. For example, cells may be affected differently (e.g., different viruses introduced), and different perturbations may be introduced into different sub-populations of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

Below, methods are exemplified using single-cell gene expression measurements. It is to be understood that this is by way of illustration and not limitation, as the present disclosure encompasses analogous methods using measurements of other cellular-components obtained from single-cells. It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

As discussed herein, gene expression in a cell can be measured by sequencing the cell and then counting the quantity of each gene transcript identified during the sequencing. In some embodiments, the gene transcripts sequenced and quantified may comprise RNA, for example mRNA. In alternative embodiments, the gene transcripts sequenced and quantified may comprise a downstream product of mRNA, for example a protein such as a transcription factor. In general, as used herein, the term “gene transcript” may be used to denote any downstream product of gene transcription or translation, including post-translational modification, and “gene expression” may be used to refer generally to any measure of gene transcripts.

Although the remainder of this description focuses on the analysis of gene transcripts and gene expression, all of the techniques described herein are equally applicable to any technique that obtains data on a single-cell basis regarding those cells. Examples include single-cell proteomics (protein expression), chromatin conformation (chromatin status), methylation, or other quantifiable epigenetic effects.

The following description provides an example general description for culturing a population of cells in vitro in order to carry out single-cell cellular-component expression measurement multiple time periods. Methods for culturing cells in vitro are known in the art. Those of skill in the art will also appreciate how this process could be modified for longer/shorter periods, for additional/fewer single-cell measurement steps, and so on.

In one embodiment, the process for culturing cells in a first cell state into cells in a second cell state includes one or more of the following steps:
Day 0: Thaw cells in the first cell state into a plate in a media suitable for growth of the cells.
Day 1: Seed cells in the first cell state into a multi-well plate. If applicable, perform additional steps to affect gene expression by cells. For example, simultaneously infect with one or more viruses to activate or knock out genes of interest.
   Perform gene expression measurement iteration t₁ for cells in the wells.
Day 1+l: Change media as needed if any additional processes are performed.
   If applicable, perform gene expression measurement iteration tₗ for cells in the wells.
Day 1+m: Change media to media appropriate to support growth of cells in the second cell state.
   If applicable, perform gene expression measurement iteration tₘ for cells in the wells.
Days 1+n, o, p, etc.: Media change as needed to support further cell state transition from the first cell state to the second cell state. If applicable, perform additional steps to affect further transition from the first cell state to the second cell state. For example, add perturbations of interest to push cells towards the second cell state.
   If applicable, perform gene expression measurement iterations tₙ, tₒ, tₚ, etc., for cells in the wells.
Day q: Perform gene expression measurement iteration t_{q} for cells in the wells and in the second state.
Collect cells into a tube and stain in suspension with antibodies matched to genes/proteins of interest, thereby sorting/identifying cells without having to lyse/destroy them. This step also can identify surface proteins that might not be seen with as much resolution in the setting of the cytoplasm. Image with a cell imaging system such as the BD Celestra flow cytometer or similar instrument by acquiring the cells from each well or tube. Quantify of number of cells per well that are in the first cell state and the number of cells per well that are in the second cell state. These steps can be used with unfixed cells.

Any aspect or embodiment disclosed herein can be combined with any other aspect or embodiment as disclosed herein.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the disclosure. Certain terms are discussed herein to provide additional guidance to the practitioner in describing the compositions, the devices, the methods and the like of aspects of the disclosure and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The term “perturbation” in reference to a cell (e.g., a perturbation of a cell or a cellular perturbation) refers to any treatment of the cell with one or more active agents capable of causing a change in the cell's lineage or cell state (or in the lineage or cell state of the cell's progeny). These active agents can be referred to as “perturbagens.” In embodiments, the perturbagen can comprise, e.g., a small molecule, a biologic, a protein, a protein combined with a small molecule, an antibody-drug conjugate (ADC), a nucleic acid, such as an siRNA or interfering RNA, a cDNA over-expressing wild-type and/or mutant shRNA, a cDNA over-expressing wild-type and/or mutant guide RNA (e.g., Cas9 system, Cas9-gRNA complex, or other gene editing system), or any combination of any of the foregoing. As used herein, a perturbagen classified as a“compound” may be a small molecule or a biologic. Also, a perturbagen classified as “overexpression of gene” may be cDNA over-expressing a wild-type gene or an mRNA encoding a wild-type gene.

In embodiments, an mRNA may comprise a modified nucleotide that promotes stability of the mRNA and/or reduces toxicity to a subject. Examples of modified nucleotides useful in the present disclosure include pseudouridine and 5-methylcytidine. Where a perturbagen is (or includes) a nucleic acid or protein described by reference to a particular sequence, it should be understood that variants with similar function and nucleic acid or amino acid identity are encompassed as well, e.g., variants with about: 1%, 2%, 3%, 4%, 5%, 6%, 7%, 8%, 9%, 10%, 11%, 12%, 13%, 14%, 15%, or more, variation, i.e., having about: 99%, 98%, 97%, 96%, 95%, 94%, 93%, 92%, 91%, 90%, 89%, 88%, 87%, 86%, or 85% identity to the reference sequence; e.g., in some embodiments, having, for example, at least: 1, 2, 3, 4, 5, 6, 7, 8, 9, 10, 11, 12, 13, 14, 15, 20, 25, 30, 35, 40, 45, 50, or more, substitutions.

The term “progenitor” in reference to a cell (e.g., a progenitor cell) refers to any cell that is capable of transitioning from one cell state to at least one other cell state. Thus, a progenitor can differentiate into one or more cell types and/or can expand into one or more types of cell populations. In embodiments, the term progenitor refers to intestinal stem cells. In embodiments, the term progenitor refers to basal cells.

As used herein, the terms “cell fate” and “cell state” are interchangeable and synonymous.

The term “subject,” refers to an individual organism such as a human or an animal. In embodiments, the subject is a mammal (e.g., a human, a non-human primate, or a non-human mammal), a vertebrate, a laboratory animal, a domesticated animal, an agricultural animal, or a companion animal. In embodiments, the subject is a human (e.g., a human patient). In embodiments, the subject is a rodent, a mouse, a rat, a hamster, a rabbit, a dog, a cat, a cow, a goat, a sheep, or a pig.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”. Likewise, the term “and/or” covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The disclosure will be further described in the following examples, which do not limit the scope of the disclosure described in the claims.

### EXAMPLES

### Example 1: Altering Goblet Cell Number In Vitro Using Mouse Small Intestinal Organoids

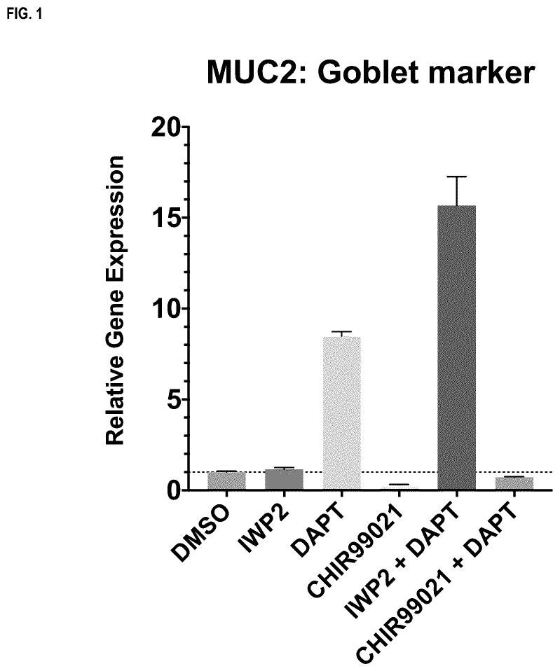

Cryopreserved mouse intestinal organoids derived from small intestine were obtained from Stem Cell Technologies. The organoids were thawed and propagated in Intesticult Growth Medium (Mouse) (purchased from Stem Cell Technologies) following the company's instructions. Briefly, approximately 100 organoid fragments/well are embedded in 6 Matrigel domes in 6-well plates and growth media is renewed every 2-3 days. The organoids are passaged every 5-6 days in 1:4 ratio. To assess the potential of the predicted perturbations to induce or reduce the goblet cell lineage during organoid differentiation, an in vitro assay was established to induce the various intestinal lineages (enterocytes, goblet cells, Paneth cells, Enteroendocrine cells) based on published data (Yin et al, Nat. Meth. 2013). Briefly, organoid fragments are embedded in Matrigel and grown in custom-made media in the presence of EGF, Noggin, Rspondin (ENR) supplemented with the indicated factors (Table 4) that are known to induce markers of the various intestinal lineages. The expression of markers and the induction of the various cell lineages was assessed by Q-PCRs after 5 days of treatments (FIG. 1). Further characterization of the organoids will be performed by immunofluorescence staining and flow cytometry. For immunofluorescence commercially available antibodies against Muc2, ChrA, and ALPI will be used.

Alternative to 3D cultures in Matrigel, organoids will be grown as 2D cultures on transwells coated with a mixture of 200 μg/ml rat tail Collagen I and 1% Matrigel, treated with ENR supplemented with the indicated factors as described. The thickness of mucus layer will be assessed by Alcian Blue/PAS stain and the presence of goblet cells will be assessed by Hematoxylin and Eosin stain and immunohistochemistry with anti-Muc2 antibodies. The histological stains will be outsourced to HistoTox Labs, Inc.

TABLE 4

| Factors | Cell lineage | Markers (Q-PCRs) |
|---|---|---|
| IWP2 | Enterocytes, Enteroendocrine Cells | ALPI, ChrA |
| DAPT | Enteroendocrine Cells, Paneth | ChrA, Lyz1 |
| IWP2 + DAPT | Goblet, Enteroendocrine Cells | Muc2, ChrA |
| CHIR99021 + DAPT | Enteroendocrine Cells, Paneth | ChrA, Lyz1 |
| CHIR99021 + Valproic acid | Stem Cells | LGR5 |

Following a similar experimental scheme, intestinal organoids will be treated with predicted compounds (Table 2) at various concentrations and their effects will be assessed on the number of goblet cells as described above.

### Example 2: Altering Goblet Cell Number In Vitro Using Human Small Intestine and Colon Organoids

Cryopreserved human colon organoids from healthy donors or patients with Crohn's or Ulcerative Colitis, or Cystic Fibrosis (F508del homozygous mutation) will be obtained from Hubrecht Organoid Technology (HUB). We will use at least 3 independent donors/group. We will use published protocols to culture and differentiate the human organoids in the presence of compounds predicted to modulate the number of Goblet cells. Similarly, to mouse organoids, we will assess the number and function of goblet cells by gene expression analyses and imaging techniques in 3D and 2D cultures.

### Example 3: Skewing Colonic Cell States to Improve Response to DSS-Induced Colitis

Adult male BALB/c mice were randomized into experimental groups using their bodyweights and allowed to acclimatize for one week. Experimental compounds (Table 2) or vehicle controls were administered daily at 10 mg/kg via intracolonic (IC) delivery starting at day 2 as outlined in Table 5 (administration schedule).

TABLE 5
Administration Schedule

| | Treatment | | | | |
|---|---|---|---|---|---|
| Group | Vehicle | Dose | Route | Regimen | Disease Induction |
| Perturbagen 3 | Olive Oil | 10 mg/kg | IC | Day −2 to Day | DSS 3%: Day 0 to |
| Vehicle (Olive oil) | Olive Oil | n/a | IC | 12 | Day 6; |
| Vehicle (water/DMSO) | Water/DMSO | n/a | IC | | ad libitum in the |
| Perturbagen 3 | Water/DMSO | 10 mg/kg | IC | | drinking water |
| Perturbagen 1 | Water/DMSO | 10 mg/kg | IC | | |
| Perturbagen 2 | Water/DMSO | 10 mg/kg | IC | | |
| Perturbagen 7 | Water/DMSO | 10 mg/kg | IC | | |
| Perturbagen 10 | Water/DMSO | 10 mg/kg | IC | | |

n/a: not applicable;
IC: intracolonic;
Groups are n = 3 or 4

On Day 0, the drinking water was replaced with a 3% dextran sulphate sodium salt (DSS) solution in distilled water. Animals were given ad libitum access to the 3% DSS solution until Day 6 included. On Day 7 until the end of the experiment on Day 12, animals were given ad libitum access to drinking water. From Day 0 until the end of the experiment on Day 12, animals were monitored daily for clinical signs of colitis to include: bodyweight loss, loose stools and/or diarrhea and presence of occult or gross blood in the stools. On Day 12, the colon (but not the small intestine) was dissected, cleaned of its content and cut longitudinally. For each animal, one half of the colon was prepared into a ‘Swiss roll’. ‘Swiss rolls’ were transferred into tissue fixative then processed for paraffin-embedding. Sections were prepared from each colon ‘Swiss rolls’ for histopathology analysis (typically one slide with two sections per colon ‘Swiss roll’). For each animal, the second half of the colon was processed for flow cytometric analysis of the epithelial fraction using EDTA to remove the epithelium and a brief collagenase digest to dissociate any clumps of cells. An aliquot of cells was stored prior to fixation/flow cytometry analysis for targeted scRNA-seq. Samples were stained for viability and the following panel of markers (Tables 6 and 7)—CD45, CD31, Ter119, EpCAM, CD117, CD24, DCLK1 and CLCA1.

TABLE 6

| Antibody | Fluorophore | Vendor | Clone |
|---|---|---|---|
| CD45 | PE | eBioscience | 30-F11 |
| CD31 | PE | Biolegend | Mec13.3 |
| Ter119 | PE | Biolegend | Ter119 |
| EpCAM | PerCPCy5.5 | eBioscience | G8.8 |
| CD117 | APC/Cy7 | Biolegend | 2BS |
| CD24 | BV421 | Biolegend | M1/69 |
| DCLK1 | AF488 | Abcam | EPR6085 |
| CLCA1 | AF647 | Abcam | EPR12554-88 |
| fixable viability dye 510 | BV510 | | |

TABLE 7
Flow Cytometry Staining and Gating Strategy

| | | Expected frequency | |
|---|---|---|---|
| Population | Gating Strategy * | (colon) | Other markers |
| Stem/TA | CD24^{lo}cKit⁻SSC^{lo} | | Lgr5 |
| Enterocytes | | 80% | Vil1 |
| Enteroendocrine | CD24^{hi} cKit⁻ | 1% | CHG-A |
| Goblet | CD24⁺cKit⁺CLCA1⁺ | 16% | Muc2 |
| Tuft | CD24⁺cKit⁺CLCA1⁻DCLK1⁺ | | |
| Paneth | CD24^{hi} cKit^{hi} | Not present/v. low | |

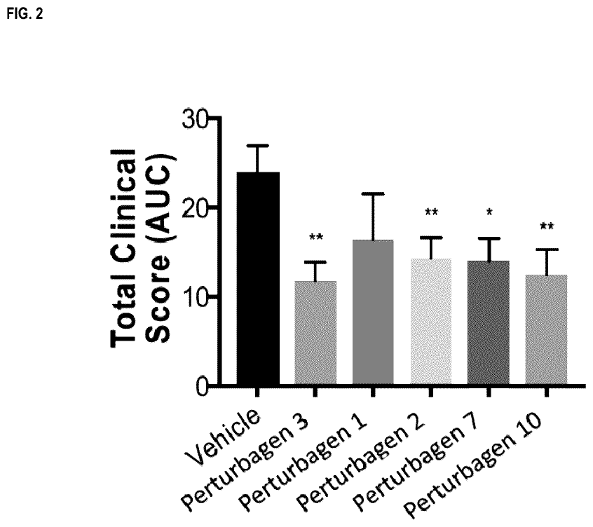

This allowed for the identification of the following intestinal cell types: Stem/progenitor cells: EPCAM+ dump (CD45/CD31/Ter119)− CD24low/− SSC low; Progenitors/Secretory lineage: EPCAM+ dump− CD24+/high; Goblet: EPCAM+ dump− CD24+/high CLCA1+; Tuft: EPCAM+ dump− CD24+/high DCLK1+; Paneth: EPCAM+ dump− CD24+ ckit high SSC high; Enteroendocrine: EPCAM+ dump− CD24+ ckit+ SSC low/mid. Cell counts were performed on the dissociated tissue to allow for quantification of the number of cell types per colon. Validation of the staining panel was performed using the small intestine and colon from 3 naïve (untreated) male BALB/c mice. All tested compounds significantly reduced the severity of DSS-induced colitis clinical scores in mice (FIG. 2).

### Example 4: Identification of Small Molecules Inhibitors of Goblet Cell Hyperplasia and Restoration of a Healthy Epithelial Layer Through Machine Learning Interrogation of Single Cell Datasets

Goblet cell hyperplasia is a major characteristic of multiple muco-obstructive airway diseases, such as COPD with chronic bronchitis, cystic fibrosis, primary ciliary dyskinesia, and non-cystic fibrosis bronchiectasis. The propensity of basal cells to differentiate toward goblet cells, at the expense of other epithelial cell types such as ciliated and club cells, leads to excessive mucus production resulting in airway obstruction, hypoxic zones, breathlessness, bacterial infections, and epithelial damage, inflammation and deterioration of lung function. Novel therapies that restore cellular homeostasis of the airway epithelium and improve muco-ciliary clearance are needed to meet the unmet needs across these diseases. A scRNA-seq dataset of IL-13 induced hyperplasia using air-liquid interface cultured human bronchial epithelial cells (HBECs) was generated and compared to an asthma patient scRNA-seq dataset (see Braga et al., Nat Med. 25:1153-1163 (2019), which is incorporated by reference herein in its entirety). Using a machine learning platform, interventions were identified that inhibit basal to goblet cell differentiation and restore ciliated and club cells in the epithelium. Eight small molecules were identified that inhibit goblet hyperplasia in IL-13 treated HBECs from COPD donors. Of these, three small molecules (Compounds A (C₂₇H₃₆N₆O₃S; MW 524.68 g/mol), B (C₃₀H₄₆O₃; MW 454.7 g/mol) and C (C₃₈H₇₂N₂O₁₂; MW 748.98 g/mol) inhibited goblet cells while promoting or maintaining differentiation to ciliated and club cells. Perturbagens referenced in this example are listed in Table 2. Furthermore, these small molecules also inhibited goblet hyperplasia in the context of other COPD-relevant models: COPD small airway cells treated with IL-13 and COPD HBECs treated with IL-17. While not wishing to be bound by any particular theory, these results suggest that small molecule activity is agnostic of inflammatory driver and airway region. Oral administration of Compound A prevented the increase of goblet hyperplasia in a murine model of IL-13 induced goblet hyperplasia, demonstrating in vivo translation of in vitro derived gene signatures. The machine learning platform identified small molecules that inhibited goblet hyperplasia and restored healthy cellular homeostasis in models of goblet hyperplasia in vitro and in vivo.

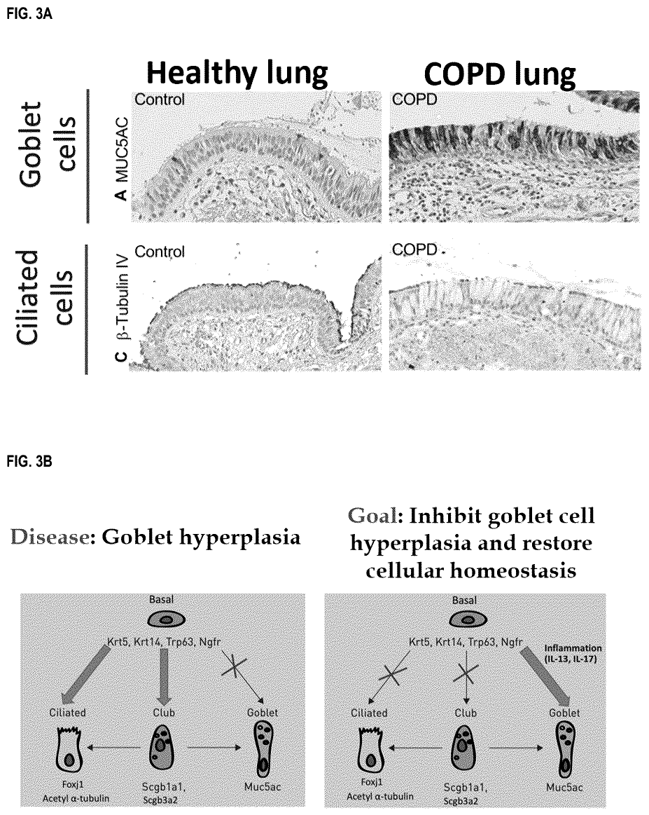

Goblet hyperplasia is a driver of disease pathology in muco-obstructive diseases such as COPD. (FIG. 3A) Inflammatory signals bias basal cells to differentiate to mucus-producing goblet cells at the expense of ciliated and club cells. Excessive mucus leads to small airway obstruction and hypoxic zones, which causes breathlessness, bacterial infections, and epithelial damage, which then drives further inflammation and deterioration of lung function (FIGS. 3A-3B). This example describes methods useful to inhibit pathological differentiation of basal cells to goblet lineage and restore healthy epithelial populations by using machine learning algorithms to identify the cellular networks driving inflammation-induced goblet differentiation (FIG. 3B).

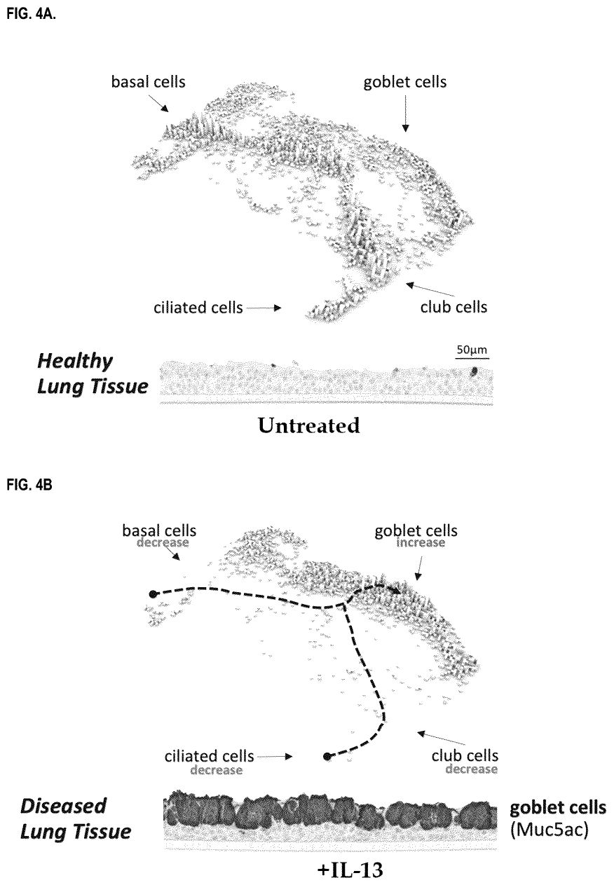

Using a scRNA-seq datasets of asthma patients (Vieira Braga et al., Nat Med. 25:1153-1163 (2019), which is incorporated by reference herein in its entirety) and of in vitro model of goblet hyperplasia, machine learning algorithms were used to predict compounds to inhibit goblet cell differentiation (FIGS. 4A-4B). Using an in vitro air-liquid interface model of the airway epithelium, human bronchial epithelial cells (HBECs) from two healthy donors were differentiated for 8 days in the presence or absence of IL-13 and sequenced at the single cell level (FIGS. 4A-4B). This single-cell RNA sequencing (scRNA-seq) analysis confirmed formation of the epithelial lineages and the induction of goblet hyperplasia by IL-13 treatment as evidenced by loss of Foxj1-expressing ciliated cells, Scgb1a1-expressing club cells, and an increase of Muc5ac-expressing goblet cells.

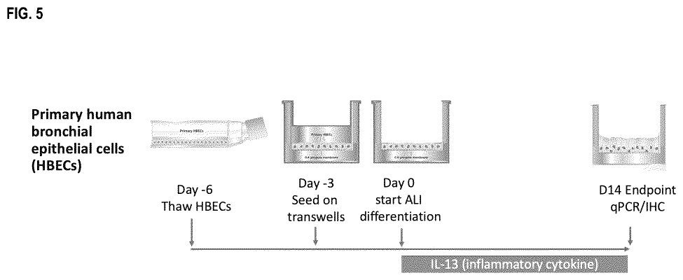

Air-liquid interface (ALI) differentiation of primary human bronchial epithelial cells (HBECs) is an accepted in vitro model of IL-13-induced goblet cell hyperplasia. HBECs were initially thawed into T75 flasks in PneumaCult maintenance media, allowed to expand, then seeded into apical chamber of transwells. After reaching confluency, medium was removed from the apical and basal chambers, and replaced only in the basal chamber with complete PneumaCult-ALI Medium to start air-liquid interface (ALI) differentiation. Cells were differentiated under ALI conditions for 14 days, in presence or absence of IL-13. Medium in the basal chamber containing IL-13 and test compounds was replenished 3 times a week, and excess mucus was washed from the apical chamber with PBS twice a week starting on day 7. At day 14, differentiation to goblet, ciliated, and club cells is assessed by histology (IHC) or qPCR, using goblet marker (Muc5ac), ciliated cell markers (Foxj1, Acetyl α-tubulin) and club cell marker (Scgb1a1) (FIG. 5).

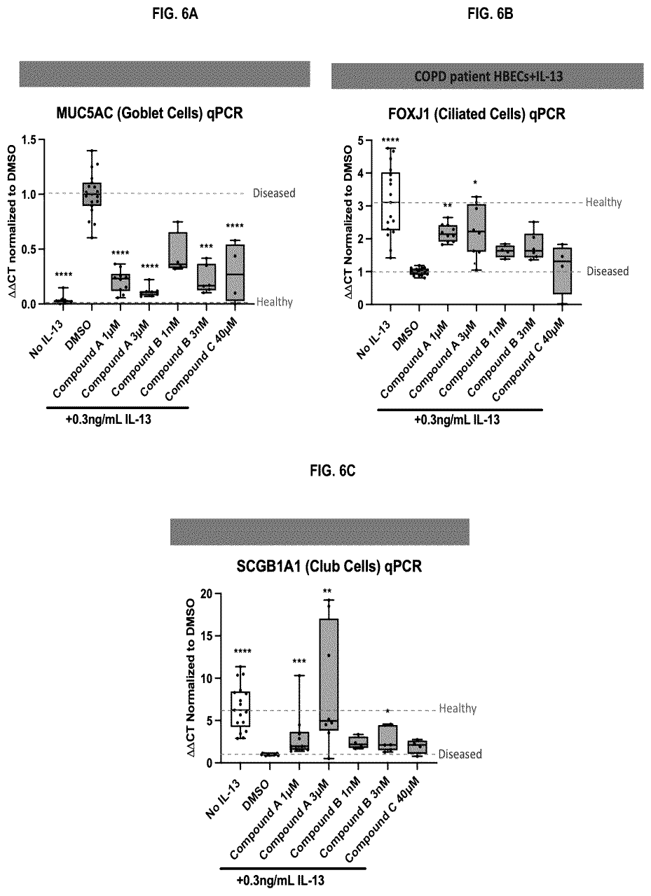

Three compounds inhibited goblet hyperplasia and maintained healthy cellular populations in COPD donor HBECs treated with IL-13 to induce goblet hyperplasia (FIGS. 6A-6C). Human bronchial epithelial cells (HBECs) from two COPD donors were differentiated in the presence of 0.3 ng/ml IL-13 and test compounds. At day 14, cultures were processed for qPCR, and evaluated for lung epithelial markers: goblet (Muc5ac), ciliated (Foxj1), club (Scgb1a1). 2\hat{ }-ΔCt values were transformed to log form and normalized to DMSO. IL-13 treatment led to an increase of disease-driving goblet cells and decrease of ciliated and club cells (FIGS. 6A-6C). Three compounds inhibited goblet hyperplasia in diseased donors while maintaining or promoting differentiation to ciliated and club cells, demonstrating the machine learning platform's ability to simultaneously manipulate multiple cell lineages to restore healthy cellular homeostasis.

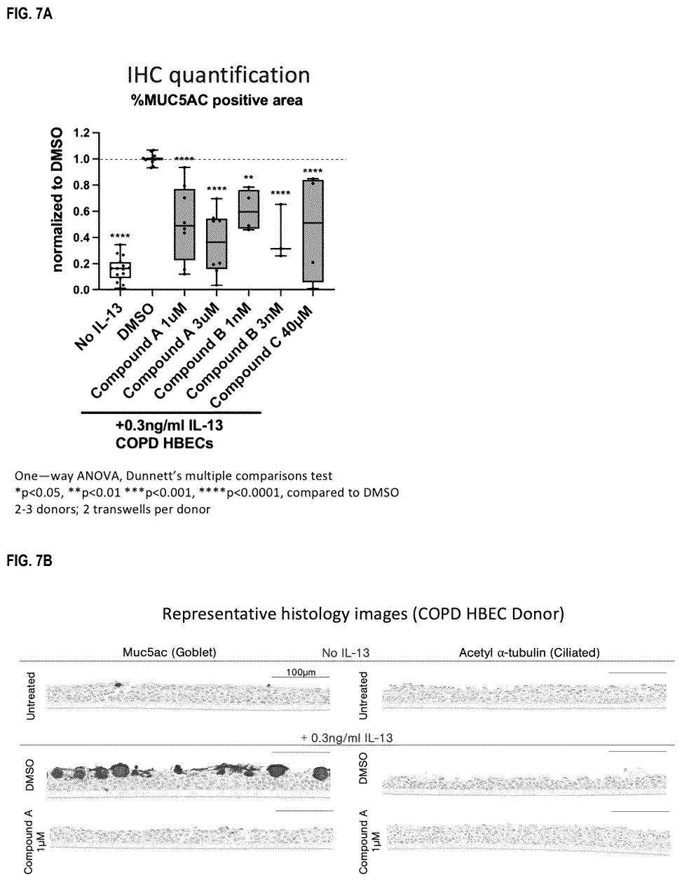

Histological analysis confirmed the inhibition of goblet hyperplasia and restoration of ciliated cell populations (FIGS. 7A-7B). Active compounds inhibit goblet hyperplasia and restore ciliated cells in COPD donor HBECs. HBECs from two or three COPD donors were differentiated in the presence of 0.3 ng/ml IL-13 and test compounds (FIG. 7A). At day 14, cultures were processed for histology, and stained for goblet (Muc5ac) and ciliated (Acetyl α-tubulin) markers (FIG. 7B). Muc5ac staining was quantified using Genie and Color Deconvolution image analysis tools on Aperio software (Leica Biosystems).

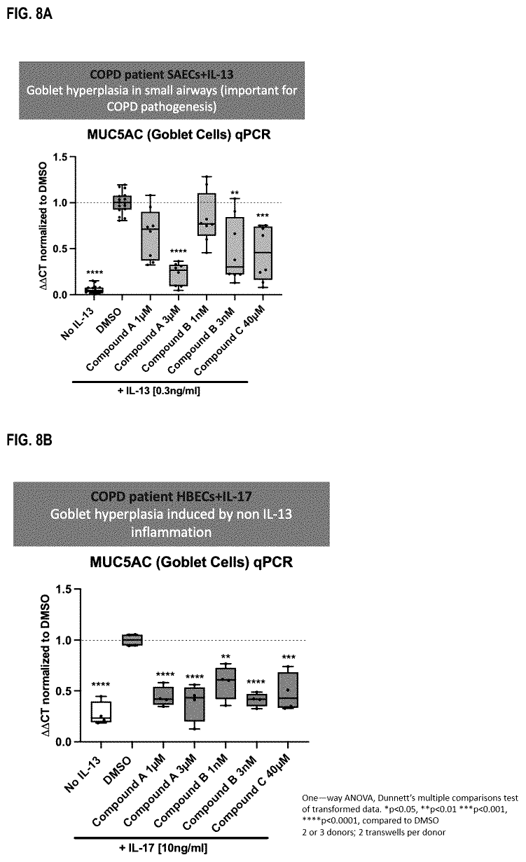

The three compounds were also found to inhibit Muc5ac in additional COPD relevant goblet hyperplasia models (FIGS. 8A-8B). While not wishing to be bound by any particular theory, efficacy in the context of both IL-17 and IL-13 induced goblet hyperplasia, as well as in small airway and bronchial epithelial cells, suggests compound activity is agnostic of inflammatory driver and airway region. To capture the different cellular contexts and inflammatory drivers of goblet hyperplasia, SAEC+IL-13 and HBEC+IL-17 orthogonal assays were developed to complement the primary assay (HBEC+IL-13). Human bronchial epithelial cells (HBECs) or small airway cells (SAEC) from COPD donors were differentiated in the presence of 10 ng/ml IL-17 or 0.3 ng/ml IL-13, respectively, and test compounds. At day 14, cultures were processed for qPCR, and evaluated for Muc5ac. 2\hat{ }-ΔCt values were transformed to log form and normalized to DMSO (FIG. 8A-B). Three compounds inhibited goblet hyperplasia in both COPD-relevant goblet hyperplasia models.

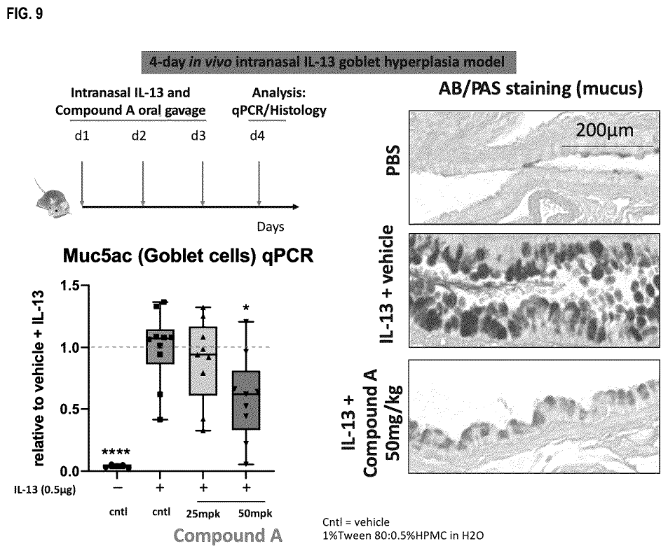

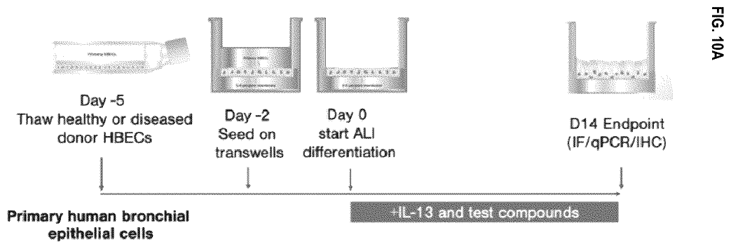

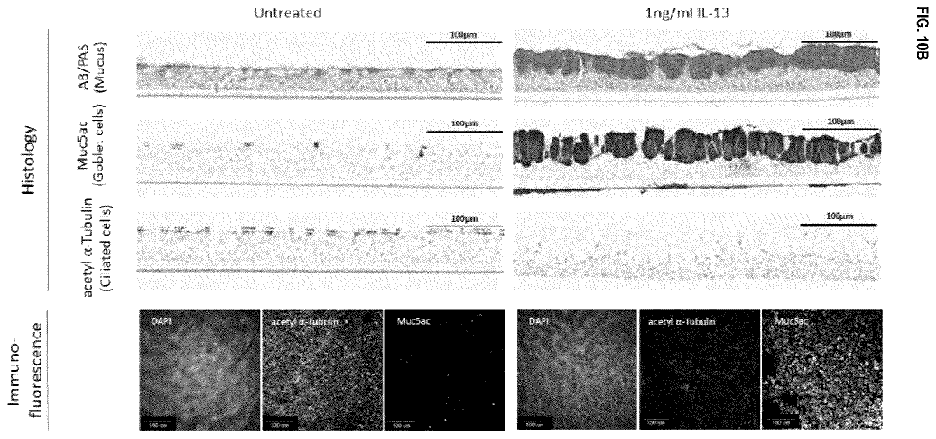

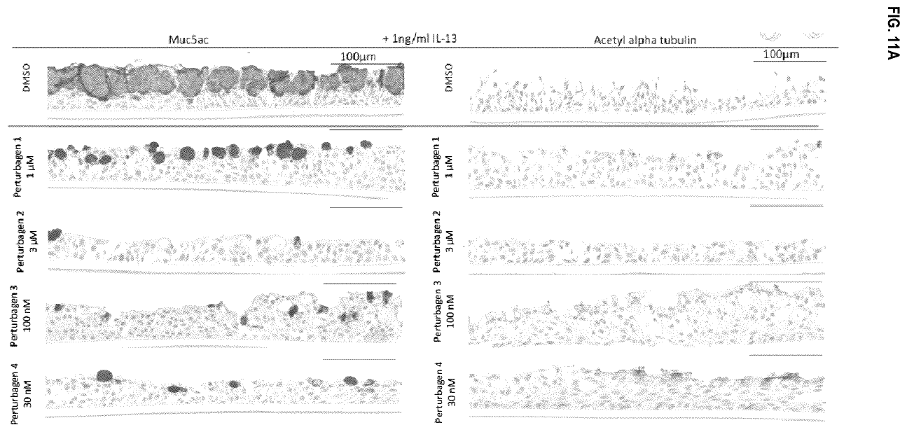

An evaluation of in vivo efficacy shows that Compound A significantly inhibits Muc5ac in an intranasal IL-13 mouse model of goblet hyperplasia (FIG. 9). 8-10 week old BALB/cJ mice were dosed with 0.5 μg IL-13 (intranasal) and Compound A or vehicle (oral gavage) for 3 days, and lobes harvested for qPCR and histology on day 4. Right lung was tied off and one lobe processed for qPCR and evaluated for Muc5ac. 2\hat{ }-ΔCt values were transformed to log form and normalized to Vehicle+IL-13 control group. Left lobe was inflated, fixed with 10% formalin, and processed for histology. AB/PAS staining was performed to visualize the presence of mucus in the airways (FIG. 9).

In summary, by applying machine learning algorithms to scRNA-seq datasets, cellular networks that drive goblet hyperplasia were identified. Three small molecules were identified and inhibited goblet cells while promoting or maintaining differentiation to ciliated and club cells. These small molecules also inhibited goblet hyperplasia in the context of additional COPD-relevant models: COPD small airway cells treated with IL-13 and COPD HBECs treated with IL-17. While not wishing to be bound by any particular theory, this result suggested that small molecule activity is agnostic of inflammatory driver and airway region. Oral administration of Compound A inhibited goblet hyperplasia in a murine model of IL-13 induced goblet hyperplasia. The machine learning algorithms identified small molecules that inhibited goblet hyperplasia and restored healthy cellular homeostasis in models of goblet hyperplasia in vitro and in vivo.

### Example 5: Experimental Methods

Air-liquid interface (ALI) differentiation of primary human bronchial epithelial cells (HBECs) is an accepted in vitro model of IL-13—induced goblet cell hyperplasia.

### Expansion of HBEC Culture

Primary HBECs (Lonza) are thawed into T75 flasks in Pneumocult Ex Plus medium (STEMCELL Technologies) and cultured for 2-3 days until 80% confluency. Cells are then dissociated for 10 min using Animal Component-Free (ACF) Cell Dissociation Kit (STEMCELL Technologies) and plated onto the apical chamber of 6.5 mm wide, 0.4 μm pore-sized Transwells (STEMCELL Technologies) at 3.3×10⁴ cells per well with PneumaCult-Ex medium on both sides of the membrane.

### ALI Culture

After the cells reach 50-80% confluency on transwells (1-2 days later), cultures are transferred to ALI conditions. Medium is removed from the apical and basal chambers and replaced only in the basal chamber with complete PneumaCult-ALI Medium (STEMCELL Technologies). Where appropriate, 1 ng/ml IL-13 (R&D) and test compounds are added to the basal chamber. Medium containing IL-13 and test compounds is replenished every 2-3 days, and excess mucus is washed from the apical chamber with 200 μl warm PBS−/− twice a week starting on day 7.

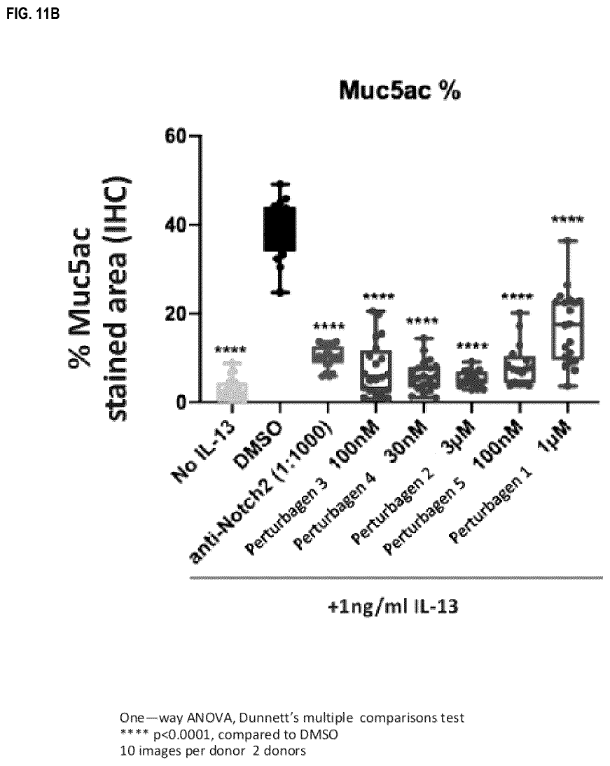

Pseudostratified airway epithelium is formed by day 14 of ALI differentiation. Goblet (Muc5ac+, AB/PAS+) and ciliated cells (Acetyl α-tubulin+) are visualized via immunofluorescence or histology. Immunofluorescence allows for a top down view of the most apical layer of cells, while histology presents a cross-section of the pseudostratified epithelium and is amenable for image quantification. In addition, qPCR of ciliated (Foxj1) and goblet (Muc5ac) marker expression can be used as a sensitive and quantitative readout of epithelial cell type distribution. Ciliated cells arise only in the untreated condition, while, conversely, goblet cells arise only in the IL-13—treated condition, modeling goblet cell hyperplasia (FIG. 10A and FIG. 10B).

### Immunofluorescence Readout

For immunofluorescence (IF) readout, transwells are fixed with 4% formaldehyde (Sigma) for 30 min and washed 3× in PBS−/−. Transwell membranes are excised with a scalpel and blocked/permeabilized for 1 hour in solution containing 2.5% goat serum (Invitrogen), 2.5% donkey serum (Sigma), 1% BSA (ThermoFisher), and 0.2% Triton X-100 (MP Biomedicals) in PBS−/−. Membranes are then incubated with primary antibodies (Muc5ac, clone 45M1, ThermoFisher and acetyl α-tubulin, clone 6-11B-1, Sigma) overnight at 4° C., washed 3× in PBS−/−, incubated with secondary antibodies (goat anti-mouse IgG1 Alexa 555 and goat anti-mouse IgG2b Alexa 647, ThermoFisher) for 45 min at room temperature, washed again, and mounted apical side down onto 48 well plates with SlowFade Diamond Antifade Mountant with DAPI (Invitrogen) and covered with 8 mm coverslips (Thomas Scientific). Plates are imaged on ImageXpress Micro 4 (Molecular Devices).

### Histology Readout

For histology readout, transwells are fixed with 4% formaldehyde (Sigma) for 4 hours, washed 3× in PBS−/−, and shipped to HistoTox Labs (HTL), Boulder CO, in PBS-filled 50 ml tubes. At HTL, samples are trimmed, placed in individually labeled cassettes, and processed per HTL SOPs for paraffin embedding. Tissue blocks are sectioned at 4 μm onto labeled slides and stained with Hematoxylin & Eosin (H&E) and Alcian Blue/Periodic Acid Schiff (AB/PAS) dyes per HTL SOPs. Immunohistochemical (IHC) staining of Formalin-Fixed Paraffin-Embedded (FFPE) is conducted on a Leica Bond Rxm using standard chromogenic methods. For antigen retrieval (HIER), slides are heated in a pH 6 Citrate based buffer for 2 hours at 70° C. (Acetyl α-Tubulin, clone EPR16772 Abcam), or pH 9 EDTA based buffer for 2 hours at 70° C. (Muc5ac, clone 45M1 ThermoFisher), followed by a 30 minute antibody incubation (acetyl α-tubulin) or 45 minute antibody incubation (Muc5ac). Antibody binding is detected using an HRP-conjugated secondary polymer, followed by chromogenic visualization with diaminobenzidine (DAB). A Hematoxylin counterstain is used to visualize nuclei. Slides are scanned and visualized on PathCore. Quantification of AB/PAS, acetyl α-tubulin, and Muc5ac staining signal is performed on MetaXpress (Molecular Devices) software as % positive area.

qPCR Readout

At day 14, transwell membranes were excised and cut in half with scalpel (VWR, 10148-884) and placed into 1.5 mL tube with 350 μL of Buffer RLT (QIAGEN, 74106) and 2-mercaptoethanol (Gibco, 21985023) added at 1:100. Lysate (350 μl) was then transferred into QIAShredder Column (QIAGEN, 79656), and centrifuged at max speed for 2 minutes at +4° C. RNA extraction was performed according to the manufacturer's instructions for RNeasy Mini Kit (QIAGEN, 74106). RNA was quantified on NanoDrop One (ThermoFisher), and cDNA generated using High-Capacity cDNA Reverse Transcription Kit (Applied Biosystems, 4368814), on the Biorad C1000 Touch Thermal Cycler.

For qPCR, target gene MUC5AC-FAM-MGB (ThermoFisher, 4331182, Hs00873651_mH) and housekeeping gene YWHAZ-VIC-MGB (ThermoFisher, 4448484, Hs03044281_g1) primers were duplexed for simultaneous detection in a single qPCR reaction. Each reaction was run in duplicate or triplicate, using TaqMan Fast Advanced Master Mix (ThermoFisher, 444456) according to manufacturer's protocols in Hard-Shell 384-well thin-wall PCR plates (BioRad HSP3805) on BioRad CFX 384 Touch Real-Time PCR Detection System. For qPCR analysis, technical replicates were normalized by housekeeping gene and averaged, and 2\hat{ }-ΔCt values were calculated.

### Example 6: Single Cell Gene Expression Profiling of In Vitro ALI Differentiation Model of Goblet Cell Hyperplasia

To determine the transcriptional changes taking place during differentiation of basal cells into ciliated and goblet cells in health and disease and elucidate the signatures of lineage commitment, we performed single cell transcriptomic profiling of HBECs from two healthy donors undergoing ALI differentiation in the presence or absence of IL-13. ALI differentiation was carried out as described above, and scRNA-seq was performed at three time points: day 0, 8, and 17 of ALI. We also validated IL-13 mediated induction of goblet cell hyperplasia in this experiment using IF staining at day 14.

Briefly, for day 0 time point, cells grown as a monolayer in T75 flasks were dissociated for 10 min at 37° C. using Animal Component-Free (ACF) Cell Dissociation Kit (STEMCELL Technologies), washed, filtered through 35 μm cell strainer (Corning) and resuspended in 0.02% BSA (ThermoFisher) in PBS−/−. For day 8 and 17 timepoints, cells grown on transwells were washed with PBS−/−, incubated with 0.05% Trypsin (ThermoFisher) in basal and apical chambers for 15 min at 37° C., quenched with RPMI/10% FBS (ThermoFisher), triturated with 1 ml pipette 10 times, filtered through 40 μm cell strainer (Corning), spun at 900 rpm for 5 min, and resuspended in 0.02% BSA in PBS−/−.

After dissociation, cell counts and viability were measured on Luna Automated Cell Counter (Logos Biosystems) using AO/PI staining, and total cell concentration was adjusted to 1.6×10⁵ cells/ml. An equal volume of 1×PBS+1% BSA+30% Optiprep was added for a final loading concentration of 8×10⁴ cells/mL in 15% Optiprep. The cells for each sample were then loaded onto the 1CellBio inDrop instrument, and emulsions made using default flow rates for beads (50 μl/hr, manually adjusting for bead packing), reverse transcription mix (250 μL/hr), cells (250 μl/hr), and oil (500 μl/hr).

Barcoded beads, microfluidic chips, droplet oil, and syringe setups used were from 1CellBio's single cell kit (Cat #10196). For each sample, two tubes of 1000 cells each were collected (4 min collection for each tube) for a target total of 2000 cells per sample. Reverse transcription and library construction were performed following Zilionis, 2017 (PMID: 27929523); with a modification of using SuperScript Ill (Invitrogen, Cat #18080044) for all reverse transcription reactions. Final libraries were checked for average size (Agilent Bioanalyzer High Sensitivity DNA chip, Cat #5067-4626) and concentration (qPCR with KAPA Library Quantification kit Cat #KK4824). Libraries were pooled and normalized at 1 nM for denature and then diluted to 1.8 pM for loading on the Illumina NextSeq 550 (NextSeq 500/550 High Output Kit v2.5 150 cycle, Cat #4232302). The same library pool was sequenced over multiple flow cells to achieve a depth of 100,000 reads per cell. Illumina custom sequencing primers for read 1 and read 2 were spiked into the standard Illumina primer wells, and the custom primer for the index read was loaded into the custom index sequencing primer well. Libraries were sequencing using 101 cycle read 1, 8 cycle index 1 read, and 50 cycle read 2. Raw sequencing data was then demultiplexed with bcl2fastq v2.20 and processed with a pipeline adapted from the umi-tools single cell tutorial (Link: umi-tools.readthedocs.io/en/latest/Single_cell_tutorial.html) to generate the gene by cell count matrix for input into downstream data analysis.

### Example 7: Testing Compounds in ALI Culture

We aimed to identify compounds that would decrease differentiation to goblet cells and simultaneously promote differentiation to ciliated cells, in order to prevent, and over-time, reverse, goblet cell hyperplasia and mucus over-secretion. We generated predictions for compounds that targeted the following transitions at day 8 and 17 time points. Perturbagens referenced in this example are listed in Table 4.
1. Block transition from IL-13—treated Basal (Basal 1, 2, or Cycling) to Goblet cell clusters, while simultaneously promoting transition from Untreated Basal (basal 1, 2, or Cycling) cluster to Ciliated (early or late) clusters.
2. Promote transition from IL-13—treated Basal to untreated Basal clusters, while simultaneously promoting transition from Basal to Ciliated clusters.
3. Promote transition from IL-13—treated Basal to untreated Basal clusters, while simultaneously inhibiting transition from Basal to Goblet cluster.

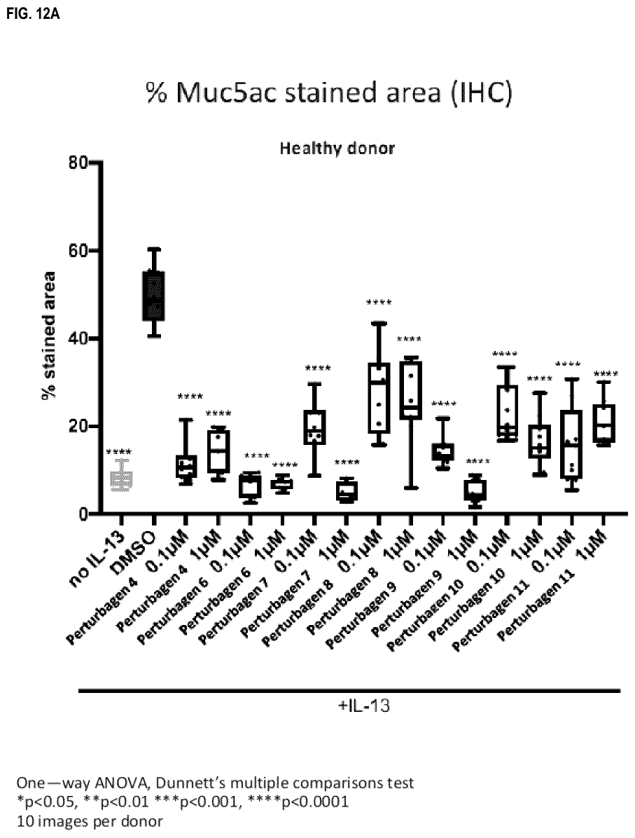

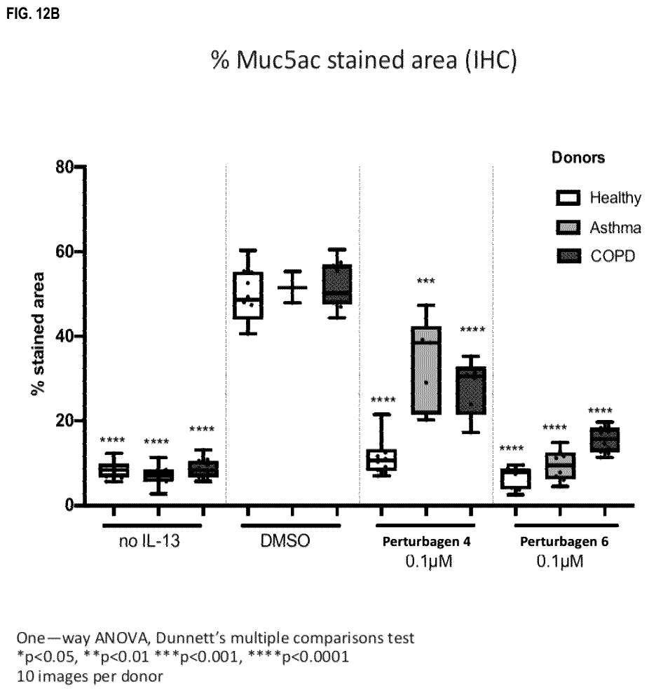

Predicted compounds were tested in the ALI differentiation assay as described above. Briefly, compounds were added on Day 0 at the start of ALI in the presence of 1 ng/ml IL-13, and medium containing compounds and IL-13, where appropriate, were replenished every 2-3 days throughout the differentiation experiment. Three (3) concentrations of compounds were tested in two healthy donors. Histology and IF and/or qPCR readouts were performed on Day 14 of ALI. Histological staining (Muc5ac, Acetyl α-Tubulin) was quantified and expressed as % positive area. Compounds that simultaneously decrease Muc5ac signal, while maintaining or increasing Acetyl α-Tubulin signal as compared to vehicle treated control were considered hits. Results show that treatment with predicted compounds rescues goblet cell hyperplasia and restores healthy cellular composition (FIG. 11A-FIG. 11B).

### Example 8: Testing Perturbagens in ALI Culture in Healthy and Diseased Cells

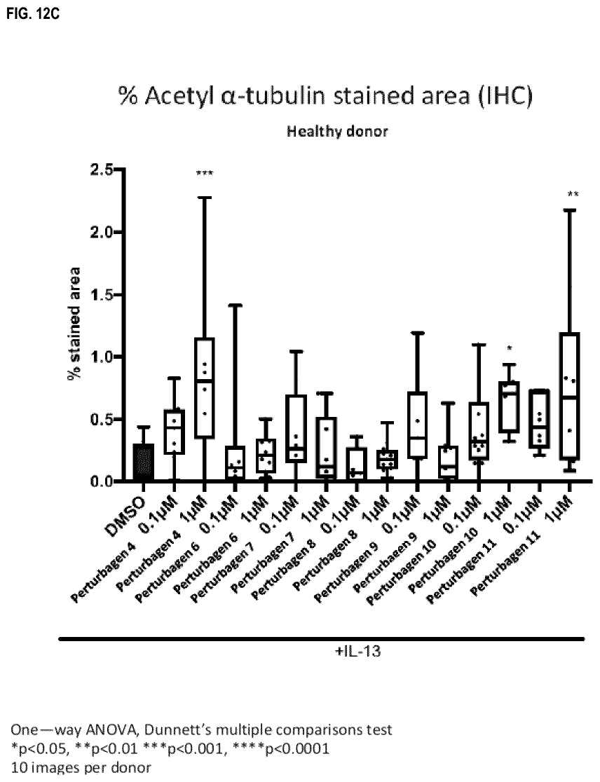

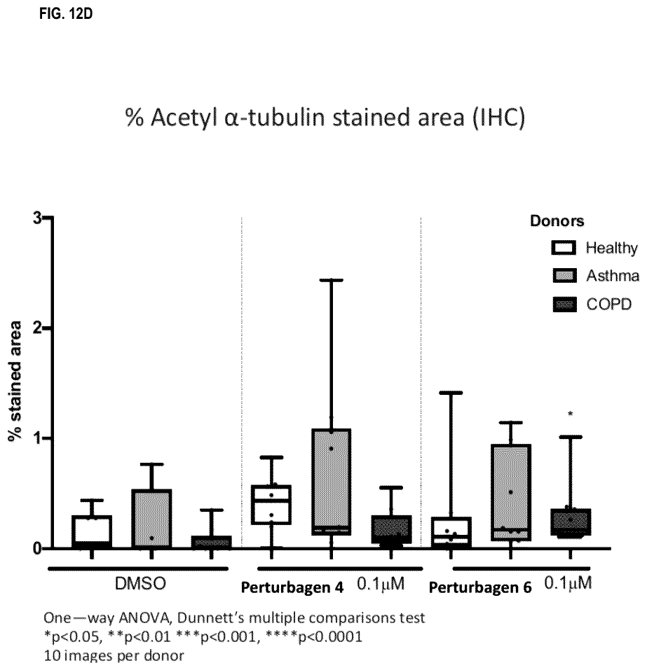

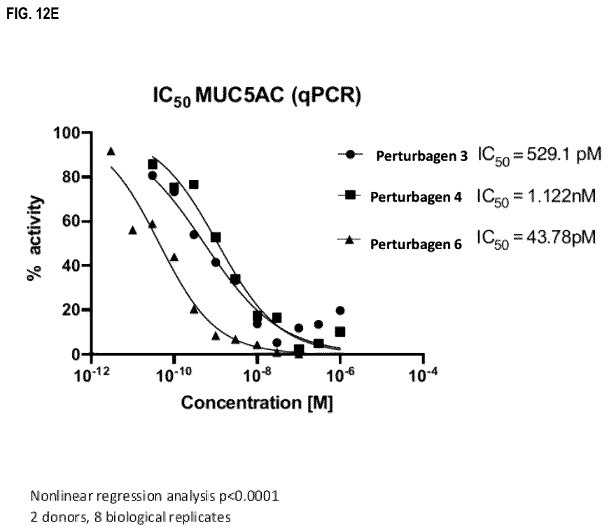

Based on the ability of Perturbagen 3, a predicted compound, to block goblet hyperplasia and restore ciliated cells in ALI+IL-13 culture, we tested additional analogs, including Perturbagen 4, Perturbagen 6, Perturbagen 7, Perturbagen 8, Perturbagen 9, Perturbagen 10, and Perturbagen 11. HBECs of healthy and diseased (asthma, COPD) donors were differentiated at ALI. Compounds were added starting on Day 0 at the start of ALI in the presence of IL-13 (0.3 ng/ml healthy donors, 1 ng/ml diseased donors). Medium containing compounds and IL-13, where appropriate, were replenished every 2-3 days throughout the differentiation experiment. At day 14 of differentiation cultures were processed for histology and stained for Muc5ac (FIG. 12A and FIG. 12B) and acetyl α-Tubulin (FIG. 12C and FIG. 12D). Histological staining (Muc5ac, Acetyl α-Tubulin) was quantified in 10 images/compound and expressed as % positive area. Compounds that simultaneously decrease Muc5ac signal, while maintaining or increasing Acetyl α-Tubulin signal as compared to vehicle treated control were considered hits. Analogs blocked goblet hyperplasia and maintained or increased ciliated cells in healthy donors; Perturbagen 6 also blocked goblet hyperplasia and maintained ciliated cells in diseased (asthma, COPD) donors. Perturbagens referenced in this example are listed in Table 4.

To compare IC50 of selected analogs, HBECs from two healthy donors were differentiated in ALI in the presence of 1 ng/ml IL-13 and a range of 10 concentrations of Perturbagen 3, Perturbagen 4, and Perturbagen 6. At day 14, cultures were lysed, RNA extracted, and cDNA generated. Goblet hyperplasia was assessed by Muc5ac qPCR. For IC50 calculation, 2\hat{ }-ΔCt values were transformed to log form and normalized. Curve fit was calculated using non-linear regression and Log IC50 of compounds were statistically compared via extra sum of squares F test. As shown in FIG. 12E, Perturbagen 6 demonstrated a more than 10-fold increase in potency over Perturbagen 3 and Perturbagen 4 (P<0.0001). Perturbagen 6 is a more potent inhibitor of goblet hyperplasia in diseased primary cells, and has a much lower calcemic potency compared to Perturbagen 3, suggesting a better safety profile.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for directing a change in cell state of a progenitor cell comprising:
contacting a population of cells comprising a progenitor cell with at least one perturbagen selected from Table 2, or a variant thereof;
wherein the at least one perturbagen is capable of altering a gene signature in the progenitor cell; and
wherein the progenitor cell is an intestinal stem cell.

2. A method for directing a change in cell state of a progenitor cell, comprising:
contacting a population of cells comprising a progenitor cell with at least one perturbagen capable of altering a gene signature in the progenitor cell;
wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and
wherein the progenitor cell is an intestinal stem cell.

3. A method for directing a change in cell state of a progenitor cell, comprising:
contacting a population of cells comprising a progenitor cell with at least one perturbagen selected from Table 2, or a variant thereof, and capable of altering a gene signature in the progenitor cell;
wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and
wherein the progenitor cell is an intestinal stem cell.

4. The method of any one of claims 1-3, wherein altering the gene signature comprises a change in expression and/or activity of one or more genes in the progenitor cell of a network module designated in the network module column of Table 1.

5. The method of any one of claims 1 to 3, wherein the change in cell state provides an increase in the number of one or more of goblet progenitors, goblet cells, Paneth cells, and enteroendocrine cells.

6. The method of claim 5, wherein the change in cell state provides an increase in the number of goblet cells.

7. The method of claim 6, wherein the increase in the number of goblet cells is relative to the number of goblets cells obtained from a population of progenitor cells that is not contacted with the at least one perturbagen or relative to the number of goblets cells obtained from a population of progenitor cells prior to contacting with the at least one perturbagen.

8. The method of any one of claims 1 to 7, wherein the number of progenitor cells is decreased.

9. The method of claim 8, wherein the decrease in the number of progenitor cells is relative to the number of progenitor cells in a population of progenitor cells that is not contacted with the at least one perturbagen or relative to the number of progenitor cells in the population prior to contacting with the at least one perturbagen.

10. The method of any one of claims 1 to 9, wherein the number of progenitor cells is increased.

11. The method of claim 10, wherein the increase in the number of progenitor cells is relative to the number of progenitor cells in a population of progenitor cells that is not contacted with the at least one perturbagen or relative to the number of progenitor cells in the population prior to contacting with the at least one perturbagen.

12. The method of any one of claims 1 to 11, wherein the number of goblet progenitors, goblet cells, Paneth cells, and/or enteroendocrine cells is increased after contacting the population of cells comprising a progenitor cell with the at least one perturbagen.

13. The method of any one of claims 1 to 12, wherein the number of goblet progenitors, Paneth cells, enteroendocrine cells, enterocyte progenitors, and/or enterocytes is decreased.

14. The method of any one of claims 1 to 11, wherein the number of goblet progenitors, goblet cells, Paneth cells, and/or enteroendocrine cells is increased.

15. The method of any one of claims 1-14, wherein the at least one perturbagen selected from Table 2, or a variant thereof, comprises at least 2, at least 3, at least 4, at least 5, at least 6, at least 7, at least 8, at least 9, at least 10, or at least 11 perturbagens selected from Table 2, or variants thereof.

16. The method of any one of claims 1-15, wherein the one or more genes are selected from the genes designated as an “up” gene in the gene directionality column of Table 1 comprises 2 or more, 3 or more, 4 or more, 5 or more, 6 or more, 7 or more, 8 or more, 9 or more, 10 genes or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, or 58 or more genes selected from the genes designated as an “up” gene in the gene directionality column of Table 1.

17. The method of claim 16, wherein the one or more genes selected from Table 1 comprises at least one of BIRC5, CCNB1, UBE2C, CDC20, CCNA2, S100A13, PMM2, GADD45B, GADD45A, RAP1GAP, TM9SF2, TMED10, STXBP1, GALE, KDELR2, ADGRE5, MTHFD2, FHL2, PYCR1, MVP, BAMBI, BAD, HMGCS1, ATP6V0B, FDFT1, HMGCR, EBP, ACLY, FGFR2, STAP2, XBP1, GFPT1, CDKN1A, HYOU1, ACBD3, COPB2, HERPUD1, NUCB2, CDC25B, BACE2, RGS2, RPN1, RAB27A, SLC35A1, RNH1, C2CD2L, ARHGAP1, SLC35B1, CRELD2, UFM1, ARFIP2, KIT, FAIM, NPDC1, TIMM17B, PDIA5, TM9SF3, and EDEM1.

18. The method of any one of claims 1-17, wherein the one or more genes are selected from the genes designated as a “down” gene in the gene directionality column of Table 1 comprises 2 or more, 3 or more, 4 or more, 5 or more, 6 or more, 7 or more, 8 or more, 9 or more, 10 genes or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or ore, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or ore, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, or 81 or more genes selected from the genes designated as a “down” gene in the gene directionality column of Table 1.

19. The method of claim 18, wherein the one or more genes selected from Table 1 comprises at least one of MIF, TRAP1, CETN3, CHEK2, RAE1, HES1, HADH, RFC2, MCM3, PCNA, HAT1, TOPBP1, EED, DDB2, PKIG, SNX7, DYRK3, CCNH, PHGDH, GLRX, CHP1, NR3C1, TSC22D3, EIF5, FOXO3, SOX4, H2AFV, GRB7, SNX6, NOTCH1, NET1, CD44, VDAC1, CREG1, PRSS23, FKBP4, MBNL1, MYC, MRPL12, CD320, G3BP1, CBR1, IFRD2, GRWD1, RPS5, TSPAN3, ADCK3, OXCT1, GPER1, PDGFA, TRIM2, APP, UGDH, PSME1, MTA1, RPA2, PGRMC1, PSIP1, HSPD1, PAICS, SDHB, NOLC1, ATP1B1, PLA2G4A, TOMM70A, ADH5, MRPL19, AARS, SOCS2, HEBP1, TIMM9, NIPSNAP1, OXA1L, MSRA, ARIDSB, BZW2, EDN1, POLR1C, CDK4, DCTD, and HSD17B10.

20. The method of any one of claims 1 to 19, wherein the change in cells state provides one or more of:
(a) increased secretion of mucus by a goblet cell and
(b) increased synthesis of one or more mucins, optionally selected from MUC2, MUC1, MUC3, or MUC17), trefoil factor peptides (TFF), resistin-like molecule β (RELMβ), and Fc-γ binding protein (FcγBP).

21. A method for inhibiting a change in cell state of a progenitor cell comprising:
contacting a population of cells comprising a progenitor cell with at least one perturbagen selected from Table 4, or a variant thereof,
wherein the at least one perturbagen is capable of altering a gene signature in the progenitor cell; and
wherein the progenitor cell is a basal cell.

22. A method for inhibiting a change in cell state of a progenitor cell, comprising:
contacting a population of cells comprising a progenitor cell with at least one perturbagen capable of altering a gene signature in the progenitor cell,
wherein altering the gene signature comprises a decrease in expression and/or activity in the progenitor cell of one or more genes designated as a “down” gene in the gene directionality column of Table 3 and
wherein the progenitor cell is a basal cell.

23. A method for inhibiting a change in cell state of a progenitor cell, comprising:
contacting a population of cells comprising a progenitor cell with at least one perturbagen selected from Table 4, or a variant thereof, and capable of altering a gene signature in the progenitor cell,
wherein altering the gene signature comprises a decrease in expression and/or activity in the progenitor cell of one or more genes selected from Table 4 and
wherein the progenitor cell is a basal cell.

24. The method of any one of claims 21-23, wherein altering the gene signature comprises an activation of a network module designated in the network module column of Table 3.

25. The method of any one of claims 21-23, wherein inhibiting the change in cell state provides:
i) a decrease in the number of goblet cells;
ii) an increase in the number of club cells; and/or
iii) an increase in the number of ciliated cells.

26. The method of claim 25, wherein the decrease in the number of goblet cells is relative to the number of basal cells or basal luminal precursor cells or club cells or ciliated cells or neuroendocrine cells obtained from a population of progenitor cells i) that is not contacted with the at least one perturbagen or ii) prior to contacting with the at least one perturbagen.

27. The method of claim 25, wherein the increase in the number of club cells is relative to the number of basal cells, goblet cells, basal luminal precursor cells, ciliated cells, and/or neuroendocrine cells obtained from a population of progenitor cells i) that is not contacted with the at least one perturbagen or ii) prior to contacting with the at least one perturbagen.

28. The method of claim 25, wherein the increase in the number of ciliated cells is relative to the number of basal cells, goblet cells, basal luminal precursor cells, club cells, and/or neuroendocrine cells obtained from a population of progenitor cells i) that is not contacted with the at least one perturbagen or ii) prior to contacting with the at least one perturbagen.

29. The method of any one of claims 21-25, wherein the number of basal cells is increased.

30. The method of claim 29, wherein the increase in the number of basal cells is relative to the number of basal cells in a population of basal cells that is not contacted with the at least one perturbagen or relative to the number of basal cells in the population prior to contacting with the at least one perturbagen.

31. The method of claim 29 or 30, wherein the number of basal luminal precursor cells, and/or goblet cells is decreased after contacting the population of cells comprising a basal cell with the at least one perturbagen.

32. The method of any one of claims 21-31, wherein the at least one perturbagen selected from Table 4, or a variant thereof, comprises at least 2, at least 3, at least 4, at least 5, at least 6, at least 7, at least 8, at least 9, at least 10, at least 11, at least 12, or 13 perturbagens selected from Table 4, or variants thereof.

33. The method of claim 32, wherein the one or more genes selected from Table 3 comprises 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more, or 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, 67 or more, or 68 or more genes designated as a “down” gene in the gene directionality column of Table 3.

34. The method of claim 32 or 33, wherein the one or more genes designated as a “down” gene in the gene directionality column of Table 3 are selected from PLP2, GAPDH, SNCA, CDH3, FKBP4, CAMSAP2, PPP1R13B, NISCH, HTRA1, ATP11B, ETS1, CPSF4, TLE1, CDK2, SESN1, GRB7, CERK, ZNF318, MYC, ELOVL6, STAMBP, EBNA1BP2, MSH6, FAH, EIF4EBP1, SLC35F2, RRP1B, G3BP1, UTP14A, DUSP3, FHL2, VPS72, ARL4C, ARPP19, CDKN1B, TP53, CRYZ, PLOD3, DDIT4, LAMA3, INPP1, CDK7, KLHL21, TIAM1, TIPARP, FOXJ3, NPC1, TUBB6, TPM1, RPA3, SFN, ST3GAL5, GMNN, ACOT9, BLMH, NNT, USP1, FKBP14, HSPB1, TBP, EPB41L2, CDCA4, TRAM2, CETN3, ETRN, PDLIM1, BRCA1, and LOXL1.

35. The method of any one of claims 21-34, wherein the change in cells state provides one or more of:
(a) decreased secretion of mucus by a goblet cell and
(b) decreased synthesis of one or more mucins, optionally selected from MUC5AC, MUC5B, MUC2, MUC4, MUC7, MUC8, and MUC19.

36. The method of any one of claims 1 to 35, wherein contacting the population of progenitor cells occurs in vitro or ex vivo or in vivo in a subject.

37. A perturbagen for use in the method of any one of claims 1 to 36.

38. A pharmaceutical composition comprising the perturbagen of claim 37.

39. A method for promoting the formation of a goblet cell, or an immediate progenitor thereof, comprising:
exposing a starting population of intestinal stem cells to a perturbation having a perturbation signature that promotes the transition of the starting population of intestinal stem cells into a M₀ cell or a goblet cell,
wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1.

40. The method of claim 39, wherein the perturbation signature comprises an increase in expression and/or activity of one or more genes in the progenitor cell of an activation of a network module designated in the network module column of Table 1.

41. A method for inhibiting the formation of a goblet cell and/or a ciliated cell, or an immediate progenitor thereof, comprising:
exposing a starting population of progenitor cells comprising at least one basal cell to a perturbation having a perturbation signature that prevents progression of a progenitor cell into and/or reduces the likelihood that a progenitor cell will progress into a goblet cell or other lineage associated progenitor thereof,
wherein the perturbation signature comprises a decreased expression and/or activity in the progenitor cells of one or more genes selected from Table 3 designated as a “down” gene in the gene directionality column of Table 3.

42. The method of claim 41, wherein the formation of goblet cells is inhibited.

43. The method of claim 42, wherein the perturbation signature comprises an activation of a network module designated in the network module column of Table 3.

44. A method for treating a disease or disorder characterized by an abnormal number or function of goblet cells, comprising:
(a) administering to a patient in need thereof a therapeutically effective amount of at least one perturbagen selected from Table 2 and/or Table 4, or a variant thereof, including combinations of the foregoing, wherein the at least one perturbagen is capable of changing a gene signature in a progenitor cell or
(b) administering to a patient in need thereof a cell, the cell having been contacted with at least one perturbagen selected from Table 2 and/or Table 4, or a variant thereof, including combinations of the foregoing, wherein the at least one perturbagen is capable of changing a gene signature in a progenitor cell.

45. The method of claim 44, wherein the disease or disorder is caused by a goblet cell deficiency.

46. The method of claim 44 or 45, wherein the disease or disorder is selected from intestinal infection, colitis, inflammatory bowel disease (IBD), ulcerative colitis, cystic fibrosis, and cancer.

47. The method of claim 44, wherein the disease or disorder is caused by an increase in the number of goblet cells or an increase in the production of mucus by goblet cells.

48. The method of claim 44 or 45, wherein the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

49. A method for treating a disease or disorder characterized by an abnormal ratio of goblet cells to intestinal stem cells and/or basal cells, comprising:
(a) administering to a patient in need thereof at least one perturbagen selected from Table 2 and/or Table 4, or a variant thereof, including combinations of the foregoing, wherein the at least one perturbagen is capable of changing a gene signature in an intestinal stem cell and/or a basal cell, or
(b) administering to a patient in need thereof a cell, the cell having been contacted with at least one perturbagen selected from Table 2 and/or Table 4, or a variant thereof, including combinations of the foregoing, wherein the at least one perturbagen is capable of changing a gene signature in an intestinal stem cell and/or a basal cell.

50. The method of claim 49, wherein the abnormal ratio comprises a decreased number of goblet cells and/or an increased number of intestinal stem cells.

51. The method of claim 49, wherein the disease or disorder is selected from intestinal infection, colitis, inflammatory bowel disease (IBD), ulcerative colitis, cystic fibrosis, and cancer.

52. The method of any one of claims 49 to 51, wherein the at least one perturbagen is capable of changing a gene signature in a progenitor cell.

53. The method of claim 49, wherein the abnormal ratio comprises a decreased number of goblet cells and/or an increased number of basal cells and/or an increased number of basal cells and/or a decreased number of goblet cells.

54. The method of claim 53, wherein the disease or disorder is selected from chronic obstructive pulmonary disease (COPD), cystic fibrosis (CF), primary ciliary dyskinesia (PCD), non-cystic fibrosis bronchiectasis (NCFB), asthma, and severe, glucocorticoid-resistant asthma.

55. The method of any one of claims 49, 53, or 54, wherein the at least one perturbagen is capable of changing a gene signature in a basal cell.

56. The method of any one of claims 44 to 55, wherein the patient is selected by steps comprising:
obtaining from the patient having the disease or disorder a sample of cells comprising at least one intestinal stem cell and/or at least one basal cell; and
contacting the sample of cells with least one perturbagen selected from Table 2 and/or Table 4, or a variant thereof, including combinations of the foregoing,
wherein the at least one perturbagen alters a gene signature in the sample of cells.

57. The method of any one of claims 44 to 55, wherein the patient is selected by steps comprising:
obtaining from a subject having the disease or disorder a sample of cells comprising at least one intestinal stem cell and/or at least one basal cell; and
contacting the sample of cells with at least one perturbagen capable of altering a gene signature in an intestinal stem cell and/or a basal cell,
wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, and/or decreases in the sample of cells the expression and/or activity of one or more genes selected from Table 3 designated as a “down” gene in the gene directionality column of Table 3.

58. The method of any one of claims 44 to 55, wherein the patient is selected by steps comprising:
obtaining from a subject having the disease or disorder a sample of cells comprising an intestinal stem cell and/or a basal cell; and
contacting the sample of cells with at least one perturbagen selected from Table 2 and/or Table 4, or a variant thereof, including combinations of the foregoing;
wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1 and/or decreases in the sample of cells the expression and/or activity of one or more genes selected from Table 3 designated as a “down” gene in the gene directionality column of Table 3.

59. The method of claim 57 or 58, wherein the perturbagen causes an increase in expression and/or activity of one or more genes in the progenitor cell of an activation of a network module designated in the network module column of Table 1 and/or the perturbation signature comprises an activation of a network module designated in the network module column of Table 3.

60. A method for selecting the patient of any one of claims 44 to 55, comprising:
obtaining from a subject having the disease or disorder a sample of cells comprising an intestinal stem cell and/or a basal cell; and
contacting the sample of cells with least one perturbagen selected from Table 2 and/or Table 4, or a variant thereof, including combinations of the foregoing,
wherein when the at least one perturbagen alters a gene signature in the sample of cells, the subject is selected as a patient.

61. A method for selecting the patient of any one of claims 51 to 55, comprising:
obtaining from a subject having the disease or disorder a sample of cells comprising an intestinal stem cell; and
contacting the sample of cells with at least one perturbagen capable of altering a gene signature in an intestinal stem cell and/or a basal cell,
wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, and/or decreases in the sample of cells the expression and/or activity of one or more genes selected from Table 3 designated as a “down” gene in the gene directionality column of Table 3, the subject is selected as a patient.

62. The method of claim 60 or 61, wherein altering the gene signature comprises a change in expression and/or activity of one or more genes in the progenitor cell of a network module designated in the network module column of Table 1 and/or the method alters a gene signature in the sample of cells, comprising activation of a network module designated in the network module column of Table 3.

63. A method for selecting the patient of any one of claims 44 to 55, comprising:
obtaining from a subject having the disease or disorder a sample of cells comprising an intestinal stem cell and/or a basal cell; and
contacting the sample of cells with at least one perturbagen selected from Table 2 and/or Table 4, or a variant thereof, including combinations of the foregoing;
wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, and/or decreases in the sample of cells the expression and/or activity of one or more genes selected from Table 3 designated as a “down” gene in the gene directionality column of Table 3, the subject is selected as a patient.

64. The method of claim 63, wherein the perturbagen causes an increase in expression and/or activity of one or more genes in the progenitor cell of an activation of a network module designated in the network module column of Table 1 and/or the method alters a gene signature in the sample of cells, comprising activation of a network module designated in the network module column of Table 3.

65. Use of the perturbagen of Table 2, or a variant thereof in the manufacture of a medicament for treating a disease or disorder characterized by an abnormal ratio of goblet cells to intestinal stem cells and/or an abnormal ratio of goblet cells to enterocytes, Paneth cells and/or enteroendocrine cells.

66. Use of the perturbagen of Table 4, or a variant thereof in the manufacture of a medicament for treating a disease or disorder characterized by an abnormal ratio of goblet cells to basal cells and/or an abnormal ratio of goblet cells to basal luminal cells, club cells, ciliated cells, and/or neuroendocrine cells.

67. A method of identifying a candidate perturbation for promoting the transition of a starting population of intestinal stem cells and/or basal cells into goblet cells or immediate progenitors thereof, the method comprising:
exposing the starting population of intestinal stem cells and/or basal cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of intestinal stem cells and/or basal cells into goblet cells or immediate progenitors thereof following exposure of the population of cells to the perturbation; and
identifying the perturbation as a candidate perturbation for promoting the transition of a population of intestinal stem cells and/or basal cells into goblet cells or immediate progenitors thereof based on the perturbation signature,
wherein the perturbation signature is an increase in expression and/or activity in the intestinal stem cell of one or more genes designated as an “up” gene in the gene directionality column of Table 1, and/or a decrease in expression and/or activity in the intestinal stem cell of one or more genes designated as a “down” gene in the gene directionality column of Table 1, and/or a decrease in expression and/or activity in the basal cell of one or more genes selected from Table 3 designated as a “down” gene in the gene directionality column of Table 3.

68. The method of claim 67, wherein the perturbation signature is an increase in expression and/or activity in the progenitor cell of an activation of a network module designated in the network module column of Table 1 and/or an activation of a network module designated in the network module column of Table 3.

69. A method for making a therapeutic agent for a disease or disorder selected from intestinal infection, colitis, inflammatory bowel disease (IBD), ulcerative colitis, cystic fibrosis, and cancer, comprising:
(a) identifying a candidate perturbation according to the method of claim 67 and
(b) formulating the candidate perturbation as a therapeutic agent for the treatment of the disease or disorder.

70. A method for making a therapeutic agent for a disease or disorder characterized by an abnormal ratio of goblet cells to basal luminal cells, club cells, ciliated cells, and/or neuroendocrine cells, comprising:
(a) identifying a candidate perturbation according to the method of claim 67; and
(b) formulating the candidate perturbation as a therapeutic agent for the treatment of the disease or disorder.

71. A method for inhibiting a change in cell state of a progenitor cell, comprising:
contacting a population of cells comprising a progenitor cell with at least one perturbagen capable of altering a gene signature in the progenitor cell,
wherein altering the gene signature comprises a decrease in expression and/or activity in the progenitor cell of one or more genes designated as a “down” gene in the gene directionality column of Table 3;
wherein the progenitor cell is a basal cell;
wherein inhibiting the change in cell state provides a decrease in the number of goblet cells; an increase in the number of club cells; and an increase in the number of ciliated cells; and
wherein the change in cells state provides one or more of:
(a) decreased secretion of mucus by a goblet cell and
(b) decreased synthesis of one or more mucins, optionally selected from MUC5AC, MUC5B, MUC2, MUC4, MUC7, MUC8, and MUC19.

72. The method of claim 71, wherein the decrease in the number of goblet cells is relative to the number of basal cells or basal luminal precursor cells or club cells or ciliated cells or neuroendocrine cells obtained from a population of progenitor cells i) that is not contacted with the at least one perturbagen or ii) prior to contacting with the at least one perturbagen.

73. The method of claim 71, wherein the increase in the number of club cells and/or ciliated cells is relative to the number of basal cells, goblet cells, basal luminal precursor cells, ciliated cells, and/or neuroendocrine cells obtained from a population of progenitor cells i) that is not contacted with the at least one perturbagen or ii) prior to contacting with the at least one perturbagen.

74. The method of claim 71, wherein the at least one perturbagen is selected from Table 4, or a variant thereof.

75. The method of claim 74, wherein the at least one perturbagen selected from Table 4, or a variant thereof, comprises at least 2, at least 3, at least 4, at least 5, at least 6, at least 7, at least 8, at least 9, at least 10, at least 11, at least 12, or 13 perturbagens selected from Table 4, or variants thereof.

76. The method of claim 75, wherein the one or more genes designated as a “down” gene in the gene directionality column of Table 3 are selected from PLP2, GAPDH, SNCA, CDH3, FKBP4, CAMSAP2, PPP1R13B, NISCH, HTRA1, ATP11B, ETS1, CPSF4, TLE1, CDK2, SESN1, GRB7, CERK, ZNF318, MYC, ELOVL6, STAMBP, EBNA1BP2, MSH6, FAH, EIF4EBP1, SLC35F2, RRP1B, G3BP1, UTP14A, DUSP3, FHL2, VPS72, ARL4C, ARPP19, CDKN1B, TP53, CRYZ, PLOD3, DDIT4, LAMA3, INPP1, CDK7, KLHL21, TIAM1, TIPARP, FOXJ3, NPC1, TUBB6, TPM1, RPA3, SFN, ST3GAL5, GMNN, ACOT9, BLMH, NNT, USP1, FKBP14, HSPB1, TBP, EPB41L2, CDCA4, TRAM2, CETN3, ETRN, PDLIM1, BRCA1, and LOXL1.
